SEQUENCE LISTING (41 sequences)

SEQ ID NO: 1-2 - skipped (2 entries)

SEQ ID NO: 3 - protein, 7 aa, synthetic construct
  REGION 1..7: note = Consensus sequence
KDQNATK

SEQ ID NO: 4 - skipped

SEQ ID NO: 5 - protein, 14 aa, synthetic construct
  REGION 1..14: note = Consensus sequence
  VARIANT 6: note = MISC_FEATURE - Xaa can be Asp or Glu
  VARIANT 7: note = MISC_FEATURE - Xaa can be any naturally occurring amino acid except proline
  VARIANT 9: note = MISC_FEATURE - Xaa can be any naturally occurring amino acid except proline
  VARIANT 10: note = MISC_FEATURE - Xaa can be Ser or Thr
GSGGGXXNXXGSGG

SEQ ID NO: 6 - protein, 19 aa, Escherichia coli
MIKFLSALILLLVTTAAQA

SEQ ID NO: 7 - protein, 21 aa, Escherichia coli
MKKTAIAIAVALAGFATVAQA

SEQ ID NO: 8 - protein, 26 aa, Escherichia coli
MKIKTGARILALSALTTMMFSASALA

SEQ ID NO: 9 - protein, 22 aa, Erwinia sp.
  source 1..22: note = Erwinia carotovorans
MKYLLPTAAAGLLLLAAQPAMA

SEQ ID NO: 10 - protein, 23 aa, Escherichia coli
MSFKKIIKAFVIMAALVSVQAHA

SEQ ID NO: 11 - protein, 28 aa, Bacillus subtilis
MFKFKKKFLVGLTAAFMSISMFSATASA

SEQ ID NO: 12 - protein, 19 aa, Escherichia coli
MKKIWLALAGLVLAFSASA

SEQ ID NO: 13 - protein, 21 aa, Escherichia coli
MKQALRVAFGFLILWASVLHA

SEQ ID NO: 14 - protein, 26 aa, Streptococcus agalactiae
MKMNKKVLLTSTMAASLLSVASVQAS

SEQ ID NO: 15 - protein, 713 aa, Campylobacter jejuni
MLKKEYLKNPYLVLFAMIILAYVFSVFCRFYWVWWASEFNEYFFNNQLMIISNDGYAFAEGARDMIAGFHQPNDLSYYGSSLSALTYWLYKITPFSFESIILYMSTFLSSLVVIPTILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAIMVKKYSQLTSNVCIVFATILTLAPVFIHIYNYKAPTVFSQNEASLLNQLKNIANREDYVVTWWDYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 16 - protein, 612 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 17 - protein, 647 aa, synthetic construct
  REGION 1..647: note = Modified EPA sequence with consensus sequences inserted atN-terminal+Y208+R274+A519
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNKDQNATKLAQQRCNLDDTWEGKIYRVLAGNPAKHDLDIKPTVISHRLHFPEGGSLAALTAHQACHLPLEAFTKDQNATKHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLKDQNATKAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 18 - protein, 666 aa, synthetic construct
  REGION 1..666: note = Modified EPA sequence with consensus sequences inserted atN-terminal+Y208+R274+A519 and E. coli DsbA signal sequence
MKKIWLALAGLVLAFSASAGSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNKDQNATKLAQQRCNLDDTWEGKIYRVLAGNPAKHDLDIKPTVISHRLHFPEGGSLAALTAHQACHLPLEAFTKDQNATKHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLKDQNATKAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 19 - DNA, 30 bp, synthetic construct
  misc_feature 1..30: note = Forward primer
  source 1..30: mol_type = other DNA
aagctagcgccgccgaggaagccttcgacc

SEQ ID NO: 20 - DNA, 50 bp, synthetic construct
  misc_feature 1..50: note = Reverse primer
  source 1..50: mol_type = other DNA
aagaattctcagtggtggtggtggtggtgcttcaggtcctcgcgcggcgg

SEQ ID NO: 21 - DNA, 768 bp, Klebsiella pneumoniae
  source 1..768: mol_type = genomic DNA
atgaagtacaatttagggtatttatttgatttacttgttgtcataacaaataaagatctaaaagtgcgctataagagcagcatgctaggctatttatggtcagtagcaaatccattgctttttgccatgatttattattttatatttaagctggtaatgagagtacaaattccaaattataccgttttcctcattaccggcttgtttccgtggcaatggtttgccagttcggccactaactcattattttcattcatcgctaacgctcaaattatcaagaagacagtttttccccggtccgtgattccgctaagtaatgtaatgatggaagggttgcattttctttgtaccatcccggttattgttgtctttctttttgtttatggcatgacgccgtccttgtcctgggtttggggtatacctctcattgctattggccaggtgattttcacctttggtgtttcaatcatcttttcaacgctgaacctgtttttccgtgacctggagcgctttgtcagtctggggattatgctgatgttttattgtacgccgattttatatgcgtctgatatgattccggaaaaatttagctggataattacctacaatccgctagcgagtatgattcttagttggcgtgatttattcatgaatgggactcttaattatgagtatatttctatactctattttacgggaatcattttgacggttgtcggtttgtctattttcaataaattaaaatatcgatttgcagagatcttgtaa

SEQ ID NO: 22 - DNA, 741 bp, Klebsiella pneumoniae
  source 1..741: mol_type = genomic DNA
atgcacccagttattaacttcagtcatgttacaaaagagtatcctctgtaccatcatattggctcaggaatcaaagatttaattttccatccgaaacgcgcttttcaattgctgaaggggcggaaatatttagctatcgaagacgtatcctttacagttggcaaaggtgaggctgttgctctgattggacgtaatggggcaggaaagagtacctctcttggcctggttgccggcgtgattaagccaactaagggaaccgtcaccactgaaggacgggtggcatcgatgcttgaactcggcggaggctttcatccggaacttaccgggcgtgagaatatttacctgaatgctactctgctgggccttcggcgtaaagaggtccagcaacgtatggaacgtattattgaattttcggaactgggagaattcatagacgagccaatcagagtgtactcaagcggaatgctagctaagttaggtttttcggtcatcagtcaggttgaaccggatattttaattattgatgaagttctggcagtaggtgatatcgcttttcaggcaaaatgtattcagaccataagagattttaagaaaagaggcgtgacaatattgtttgttagccacaatatgagtgacgttgaaaaaatctgcgacagagtcatctggatcgaaaatcataggctcagagaagtggggtctgcagagcgaatcattgaactgtacaagcaagcaatggcttaa

SEQ ID NO: 23 - DNA, 1896 bp, Klebsiella pneumoniae
  source 1..1896: mol_type = genomic DNA
atgaacaatagcgttaaaatctataccagccaccataagcctagtgcttttcttaatgctgcaattatcaaacctctgcatgtcggcaaagctaattcttgtaatgaaattggttgtccaggagatgacactggcgataatatttcctttaagaatccgttttattgcgaacttactgcgcattattgggtttggaaaaacgaagagctggcagactatgtcggtttcatgcactatcgccgtcatcttaatttttccgaaaaacaaactttttctgaggatacgtggggggtcgtgaaccatccatgcattgatgaagaatatgaggagatctttggattaaacgaagaaacaattcaacggtgtgtcgaaggtattgacatcttgctgcccaaaaaatggtctgtcactgcggcgggaagtaaaaataattacgatcactatgaacgaggtgaatacttacacattcgtgattatcaggctgccattgccaccgttgaaaaactatatccagagtatagcacggcaataaaaacgtttaatgatgccagtgatggctattacacaaatatgtttgtcatgcgcaaagatatttttgttaactattctgagtggctcttttctattctggataatctcgaagatgccatctcgatgaacaattataatgctcaggaaaaacgcgttattgggcatatagcagaacggctgtttaatatttacattattaagttgcaacaagatggtgagcttaaggtaaaagaattacagcgtacttttgtcagcaatgaaacattcaatggtgcactgaatccagtttttgattctgcggttccagtggttatcagtttcgatgataattacgcagtcagcggtggtgcattaattaattccattgtccggcatgcggataaaaataaaaattatgatatcgtcgtgctcgaaaacaaagtaagctatttgaataaaacgcggttagtaaatctaacctcggctcatccgaatatttctcttcgtttttttgacgttaatgccttcactgaaataaacggtgtgcatacccgagcgcattttagcgcatcaacgtatgcccgtctttttattcctcaactgttcagacgatacgataaagtcgtatttattgattcggataccgttgtaaaggctgacctgggtgaactgcttgatgtccctctgggcaacaatttagttgcagcggttaaggatatcgtcatggaaggttttgtaaaattttctgcaatgtcggcatcagatgatggcgttatgccggcaggcgaatatttacagaaaaccttaaatatgaataaccctgatgaatattttcaggcagggattattgtttttaatgtcaaacaaatggtcgaagaaaatacttttgctgaattgatgcgggtattaaaggcaaaaaaatactggttcctcgaccaggatatcatgaataaagtattctactctcgagtcacatttctgccattagagtggaacgtttatcatggtaatggcaacacggatgatttcttccctaatcttaagtttgcaacgtatatgaaatttttagcagctcgcaagaagcctaaaatgattcattatgcgggtgagaacaaaccatggaataccgaaaaagtcgatttttatgacgactttattgaaaacatcgctaacactccatgggagatggaaatctataaacgtcagatgtcgttagcggcttcgattggtttaacccatagcgagccgcaacaacaaatcttgttccagaccaaaatcaagaacgtactgatgccttatgttaataaatatgcaccaataggcacgccaagaagaaacatgatgactaaatattattacaaagtacgccgtgctattcttggataa

SEQ ID NO: 24 - DNA, 1155 bp, Klebsiella pneumoniae
  source 1..1155: mol_type = genomic DNA
atgaaaagaaaaaaaatattgatcgtaggcgctggcttctctggtgcagttatcggtcgccaacttgctgagaagggacatcaagtccatattatcgatcagcgtgatcatattggggggaattcttatgatgcacgcgactctgaaacgaatgtgatggtacatgtttatggaccccatattttccatactgacaatgaatcagtgtggaactatgtcaacaagcatgcagagatgatgccctatgtgaaccgggttaaagcgacagttaatggtcaggtattttccctgcctattaatttgcatactatcaatcagtttttctcaaaaacttgttcgcctgatgaggccagagcgctcattgctgagaaaggggacagcactattgctgatccacaaacttttgaagagcaagcgttacgctttattggtaaagagttatatgaggccttttttaaaggatatacgattaaacagtgggggatgcaaccctcggaactgcccgcatctattcttaaacgtcttcctgttcgttttaactatgacgataattattttaaccacaaatttcagggcatgccgaaatgtggttatacgcagatgattaagtcaattcttaagcatgagaatatcaaggttgacttacagcgggaatttatcgttgacgagcgaactcattacgatcacgtattctatagcggtccattagatgcgttttatggctaccaatatggccgtctgggctatcgaacattagattttaaaaagtttatctatcagggtgattaccagggatgcgcagtgatgaactattgttctgtggatgtgccctatactcgcatcactgaacataaatatttttctccctgggaacaacacgacggctctgtttgttataaagagtatagccgtgcttgtgaagaaaatgatattccttactatcctattcgccagatgggagagatggctcttcttgaaaaatatttgtcattggccgagaatgaaaccaacatcacttttgtcggtcgtcttggaacctaccgttaccttgatatggatgtgaccatcgccgaagcattgaaaacggcagaagtctatttaaattcactcactgaaaatcagccaatgcctgtgtttacggtttctgtacgatga

SEQ ID NO: 25 - DNA, 894 bp, Klebsiella pneumoniae
  source 1..894: mol_type = genomic DNA
atgaaatatacggcattgatagtgacattcaatcgtctcggcaaactgaaaaaaacggttgaagagaccctcaaacttgaattcactaatattgttattgtcaataacgggtccacggatgggacccaagcctggctttcgtcaattgttgatacacgagtcattgtattaaccctcaccgagaataccggtggggcggggggctttaaaaccggtagtcagtatatctgtgaacagctggcaagtgattgggtatttttctacgatgacgatgcttacccctatccagacacgttgaagtccttttcacagctggataagcagggatgtcgggtatttagtggactggtgaaagatccgcaaggaaaaccgtgtccgatgaatatgccgttctcgcgtgtgccaacttcccttggcgacactgtacgctatttacgctaccctgcagagtttatcccggcagctaatcgttctatgttcgtacaaacggtttcatttgttgggatggtcatacatcgtgatctgctcgcgaccagtcttgaccacatccatgaacagctctttatctactttgatgatctttactttggctatcagctatcattagctggtgagcaaattatgtatagcccggagttgcttttttatcatgatgtgagtattcagggcaaacttattgcccctgaatggaaggtttactatctatgccgtaatttgatcctgtcgaagaaaatattccagaaaaatgccgtatatagcaattcagcgatagcgatacgcatcctaaaatatatattaatcctgccatggcaacgtcaaaaatattcctatatgaaatttattcttcgtggaatttcacatggcataaaaggtattagtggtaagtatcattaa

SEQ ID NO: 26 - DNA, 1131 bp, Klebsiella pneumoniae
  source 1..1131: mol_type = genomic DNA
atgagaaaattgtgttatttcataaattcggattggtacttcgatttacactggatcgatcgtgccatcgcctcccgtgatgcaggttatgagattcacatcatcagccattttattgatgacaacataataaataaatttaaaacatttggctttatttgccataatgttactcttgatgctcaatcttttaatgcattagttttctttcgtacttaccatgatgtgcaaaaaattattaaaaatataaaaccggatctcttgcattgcatcactatcaagccatgtttgattggtggtgtgctcgcgaagaaatttaatctgccggtcatcgtaagttttgttgggcttggaagagtattttcttctgacagcatgcctttaaaattattgcggcagtttactattgctgcatataaatatattgccagtaataagcgctgtatatttatgtttgaacatgaccgcgacagaaaaaaactggctaagttggttggactcgaagaacaacagactattgttattgatggtgcaggcattaatccagagatatacaaatattctcttgaacaggatcacgatgtccctgttgtattgtttgccagccgtatgttgtggagtaaaggactgggcgacttaattgaagcgaagaaaatattacgcagtaagaatattcactttactttgaatgttgctggaattctggtcgaaaatgataaagatgcaatttcccttcaggtcattgaaaattggcatcagcaaggattaattaactggttaggtcgttcgaataacgtttgcgatcttattgagcaatcaaatatcgttgctttgccgtcagtttattctgaaggtgttccgcgaattcttctggaagcatcttctgtggggcgcgcttgtattgcttatgatgttggtggttgtgatagccttattattgataacgataatggaattattgttaaaagcaattcacctgaagagctggctgataaacttgcctttttgcttagcaatcctaaagcacgcgttgaaatgggtattaaggggaggaaacgtatacaagataaattttctagtgttatgattatcgataaaacattgcaaatatatcatgatgtagttcgatga

SEQ ID NO: 27 - DNA, 378 bp, Klebsiella pneumoniae
  source 1..378: mol_type = genomic DNA
atgccaagttcaggcccattatggcaactaatgaaatatgggttagttgggatagtcaatacactaattacggcagttgtaattttcctgctaatgcatttgggtcttggcatttatctgtccaatgcgatgggttatgttgtaggtattgttttcagctttatagcaaacacaatatttacatttacgcaaccaatcagtatcaatagactaataaaatttttatgtgtttgcttcatttgttatgtggcaaatatcattgtcataaaaatatttttcgtttttatgccagaaaaaatatattcagcacaaatccttgggatgttcacatacactatcacaggttttattttaaacaagttctgggcgatgaaatga

SEQ ID NO: 28 - DNA, 981 bp, Klebsiella pneumoniae
  source 1..981: mol_type = genomic DNA
atgacaacctcaactgatataaaaagcactccttctttagctattgtggtaccttgctataatgaacaagaggcttttcctttctgtctcgaaaagctttcgaatgtactaaattcattgatagccagaaataaaattaataacaatagttatcttttgtttgtcgatgatggtagtcgtgacaatacttgggcacaaattaaagatgcctcgaccgcttatcactatgtgcgaggaataaaattatcaagaaataaaggacatcaaattgcgttgatggcagggttacgctcggtcgatacagacgtaagcattagcatcgatgcggatctacaagacgatgtaaattgcatcgaaaaaatgattgacgcttacagccagggatatgacatagtatacggcgtaagaggtaatcgagacagtgacacgttttttaaacgtacaacagctaatgcattttacgcaataatgtcccacttgggagtaaatcaaactccaaatcatgcagattatcgattattaagtaatcgagcattggaggctcttaaacaatataaagagcaaaatatatatttacgtggattagtgcctcttgtgggatacccctcgatcgaggtgcaatatagccgtgaagaaagaattgcaggtgaatcaaaatatccaattaaaaaaatgcttgcgctggctctcgagggaattacctcattatcagttacaccgttacgaattatagctatgacaggttttataacttgcatcatatcaaccatcgctgcgatttatgctttaattcaaaaaacaacaggtactacagttgagggatggacatcagtcatgatcgctatattctttcttggcggcgtgcaaatgctttctttaggtattataggagaatatgtcggaaaaatttatatagagacgaaaaatagacctaaatatttcattgacgaaagcgtaggtaatgatagcaatggaaaataa

SEQ ID NO: 29 - DNA, 1461 bp, Klebsiella pneumoniae
  source 1..1461: mol_type = genomic DNA
atgcaaaatctgatcaatcctttagcagagggaaataaaaaaaacgtttacattttttatttctttttgcttatgttaacattttcaccggtaattttcttttcatatgcattttcagacgactggtcaacactctttgatgctataacaagaaacggctcttcgtttcagtgggatgtccaatctggtcgtcccgtttatgctgtgttccgttactatggaaaaatgttaattaatgatatttcttcattttcgtatttgcggctttttaatatattaagtcttgttgtcttaagttgttttatttacaacttcatagacagcagaaaaatatttgataaccccgtattcaaaataacatttccgctgttaatttgcttactccctgcgtttcaagtttatgcttcatgggcaacatgtttcccgttcactatttcagtattgctggcaggtattagttataataaatgtttcccacattcgaagcagcggtcgtcattgtcagaaaaattagcatccattgttgtcttatgggtggcatttgcaatatatcaaccgacagcaattacattcttattcttttttatgcttgatagttgtataaaaaaagaaagtagtttaactgtgaaaaaagttgcgacatgttttatcattttagttatcggtgttgcaggcagttttataatgtcaaaagtacttcctgtctggctatatggggaatcattatcgagagccgagttaaccgcagatatcggtggaaagatgaaatggttcataaatgaatcactaataaacgctgtaaataactataacatacaaccagtaaaaatatattcttggttctcctcgcttgcaattttaatcggcttatacactatttttgtgggaaaatcaggcagatggaaaacgttcatagtcatagcgatcgggataggttcctacgctccaaatttagcgacaaaagagaattgggcagcattccgctcgttagtggccttagaacttattatatcaactctatttcttattggcataaatagccttgtcagtagaatttttaagcaagcatttgtctggcctcttatcgctttaacaattatgataatagctcagtataatattataaatggatttattattcctcaacgctctgaaattcaggcacttgctgcggaaataactaataaaatacctaagaattacacaggaaaattaatgttcgatctcacagatcccgcttacaatgcctttacaaaaacacagagatatgatgaatttgggaatatttcattagcagcaccctgggcgctcaaaggtatggctgaagagatcagaattatgaaaggatttaatttcaaactatctaacaacgttatagtttctgagaccaatcgatgtattgatgattgtatggttatcaaaacgtcagatgcaatgcgaaggtcaacgataaattattag

SEQ ID NO: 30 - DNA, 2223 bp, Klebsiella pneumoniae
  source 1..2223: mol_type = genomic DNA
atgaagaaaattcttataatgacgccggacattgaggggcctgtccgtaacggcggtattggtactgctttcactgcccttgccactactttggcaaaaaaggggtatgatgttgatgtattgtatacatgtggcgactattctgaatcatctgtatcgaaatttagcgactggtcacgtatttatagtacctttggtatcaatctgctaagaaccggactgataaaagagattaatattgatgcaccgtattttagaaggaaaagttattcaatttatctctggttgaaagaaaataacacctatgacactgttatttcttgtgagtggcaggcagatctttattacactttattaagcaaaaagaatggaacggattttgaaaatacaaagttcattgtaaatactcacagttcaacgttatgggctgatgaaggtaattaccagcttccatatgatcagaaccatcttgaactctattatatggagaaaatggtggttgaaatggcggatgaagttgttagtccgtctcagtatttaattgattggatgttgagtaagcactggaatgttcctgaagaacgtcatgtaattttaaattgcgagccatttcaagggtttgtgacgagagatgatgttacagttaaaataaatgaaaagccagcttctggcgttgagcttgtatttttcggccgccttgaaacccgtaaaggacttgacatattcctgcgtgcattaagaaaactatctgatgaagataaagagagcatttctggagtaaccttcctcggaaaaaatgtcactatggggaaaactgattcatttacttatattatgaatcagactaaaaatttgggactcgcagttaatgtcatcagcgactatgatcgtaccaacgctaatgaatatataaaaagaaaaaatgtattagtcatcattccatcacttgtagaaaactcaccctatactgtttatgaatgtttgattaataacgttaattttctcgcttcaaacgttggtggaattccagagcttattccgcaggagcatcatgcggaagttctatttattcctacacctgccgatttatacggaaaaatccactatcgcttaaaaaatataaatataaaaccagggcttgctgaatcacaagacaatattaaagaagcttggtttgtcgcagttgaacgaaaaaacaaccgcacattcaagaaaatcgatgaagctaacagcccgttagttagcgtgtgtataactcacttcgaacgtcaccatttgcttcagcaagcactcgcatcaataaaatctcagacgtaccaaaatattgaggtcatcttggttgatgatggaagtacgacagaagattctcatcgttatttgaatctcatcgagaatgattttaactctcgaggctggaaaattgtccgtagttctaataactatctgggtgctgcaaggaatttggctgcgcgacacgcctctggcgaatatctgatgtttatggacgatgataatgttgctaagccttttgaggtagaaacgtttgttactgcagcattaaactctggggccgatgtgttaaccacaccaagcgatcttatttttggtgaggagttcccttctccgttccgtaaaatgacgcactgctggcttccgttagggcctgatttaaatatcgccagctttagtaactgctttggcgatgctaatgcgctgatcagaaaagaggttttcgaaaaagtaggcggatttactgaagattacggtttaggtcatgaagactgggagttttttgccaaaatatcattacagggatataaattgcaaatcgtcccggaacctctattttggtatagagttgcaaactccggcatgttgttaagtggaaataagagtaaaaataactaccgcagtttccgtccttttatggatgagaatgttaaatataactatgcaatggggttgataccttcctacctcgagaagattcaagaacttgagagtgaagtgaatcgcttgcggagcatcaatggtggtcattctgtcagtaacgagttacaacttttaaataataaggttgatggtcttatttctcagcaaagagatggctgggcccatgaccgttttaatgctctgtatgaagcaattcatgtccaaggcgcaaaacgaggcaccagcctggttcgccgggttgcccggaaagtgaaatcaatgttaaaataa

SEQ ID NO: 31 - DNA, 810 bp, Klebsiella pneumoniae
  source 1..810: mol_type = genomic DNA
atgaccaatatgaagttaaaatttgatttgcttctaaaatcttatcatctatctcatcgatttgtctataaggcaaaccctggtaatgctggtgatggtgtaattgcatctgcgacgtatgacttttttgaacgaaatgctcttacctatatcccttacagagatggcgagcgctacagttctgaaactgatattttaatttttggaggcggaggaaacctgatagaaggattgtattctgaaggtcatgactttatccagaataatattgggaagtttcataaagtaataataatgccgtcgacaatcagagggtatagcgatttattcatcaacaatattgataagtttgttgttttttgtcgcgaaaatatcaccttcgattatattaaatctctcaactacgaaccaaacaagaacgtattcattactgatgatatggcattttatctcgatcttaataaatacctgtcacttaaacccgtctataaaaaacaggccaactgcttcagaacggactccgaatctctaactggagactacaaagaaaacaatcatgatatttcgctcacctggaatggcgattattgggataatgaatttctggcgcgtaattctacccgttgcatgataaactttcttgaagagtataaagttgtcaataccgacaggctgcatgtggcaattttagcatctctgcttggcaaagaagtcaacttctatcctaactcatattacaaaaatgaagctgtttacaattattcactttttaatcgttatccaaaaacatgctttattacggcaagttga

SEQ ID NO: 32 - DNA, 1416 bp, Klebsiella pneumoniae
  source 1..1416: mol_type = genomic DNA
atgttgcttcctgtgatcatggctggtggtaccggcagtcgtctctggccgatgtctcgcgagctttacccgaaacagttcctccgcctgttcgggcagaactccatgctgcaggaaaccatcacccgactctcgggccttgaaatccatgaaccgatggtcatctgtaacgaagagcaccgcttcctggtggccgaacagctgcgccagctcaacaagctgtcgaacaacattattcttgagccggtcgggcgcaacaccgccccggccatcgccctggcggccctccaggccacccgccacggcgacgacccgctgatgctggtcctcgccgccgaccatatcatcaataaccagccggtcttccacgacgccatccgcgtcgccgagcagtatgccgatgaaggccatctggtcaccttcggtatcgtgccgaacgccccggaaaccggctacggctacatccagcgcggcgtggccctcaccgacagcgcccacaccccgtaccaggtggcccgcttcgtggagaagccggaccgcgagcgcgccgaggcctacctcgcctccggggagtactactggaacagcggcatgtttatgttccgcgccaaaaaatacctctccgagctggccaaattccgcccggatatcctcgaagcctgccaggccgcggtcaatgccgccgataacggcagcgacttcatcagcatcccgcatgacattttctgtgagtgcccggacgagtccgtggactacgcggtgatggagaaaaccgccgacgcggtggtggtcggtctcgatgccgactggagcgacgtcggctcctggtccgccctgtgggaggtcagcccgaaagatgagcagggtaacgtcctcagcggcgacgcgtgggtgcacaacagcgaaaactgctacatcaacagcgacgagaagctggtggcggccatcggcgtggagaacctggtgattgtcagcaccaaggacgccgtgctggtgatgaaccgtgagcgttcccaggacgtgaagaaggcggtcgagttcctcaagcagaaccagcgcagcgagtacaagcgccaccgcgagatttaccgtccctggggccgctgcgacgtggtggtccagaccccgcgcttcaacgtcaaccgtattacggtgaaaccgggcggcgccttctcgatgcagatgcaccaccaccgtgccgagcactgggtcattctcgccggcaccggccaggtgacggtcaacggcaagcagttcctgctgaccgagaaccagtccacctttattccgattggcgccgagcacagcctggaaaacccgggccgcattccgctggaagtgctggagatccagtcggggtcgtacctcggcgaggacgacattattcgtattaaagaccagtatggtcgttgctaa

SEQ ID NO: 33 - DNA, 1383 bp, Klebsiella pneumoniae
  source 1..1383: mol_type = genomic DNA
atgacacagttaacatgctttaaggcttatgacatccgtggtgaactgggcgaggagctgaacgaggacatcgcctaccgtatcggccgcgcctatggcgaatttctgaaacccgggaagatagtggtggggggcgatgtgcgcctcaccagcgagtcgctgaagctggcgctggcccgcgggctgatggacgccggcaccgacgtgctggatattggcctgagcggcacggaagagatttacttcgccactttccacctcggggtggacggcggtatcgaggtgacggcgagccataacccgatgaactacaacggcatgaagctggtgcgcgagaacgcgaagcccatcagcggcgacaccggcctgcgggatatccagcgcctggcggaggagaatcagttcgcgccggtagacccggcgcgtcgcgggaccctgcgccagatatcggtgctgaaggagtacgtcgaccacctgatgggctatgtggacctggcgaacttcacccgtccgctgaagctggtggtgaactccggcaacggggcggcggggcacgtgattgatgaagtggagaaacgcttcgcggcggccggggcgccggtgacctttatcaaggtgcatcaccagccggacggccatttcccgaacggtatcccgaacccgctgctgccggagtgccgccaggacaccgccgacgcggtgcgtgcgcatcaggcggacatggggatcgcctttgacggcgacttcgaccgctgcttcctgttcgatgacgaggcgtcgtttatcgaggggtactacattgtcggcctgctggcggaggcgttcctgcagaaacagccgggggcgaaaatcattcacgacccgcgtctgacgtggaacacggtggacatcgtgacccgcagcggcggccagccggtgatgtcgaagacggggcatgcgttcatcaaggagcggatgcgccaggaagacgccatctacggcggggaaatgagtgcgcaccattacttccgcgacttcgcctactgcgacagcgggatgatcccgtggctgctggtggcggagctgctgtgcctgaagaacagttcgctgaaatcgctggtggcggaccgccaggcggcgttcccggcgtcgggggagatcaaccgcaagctggggaatgcggcggaggcgatagcgcgcatccgggcgcagtatgagccggccgccgcacacatcgacacaacggacggtatcagtattgaataccctgagtggcgctttaacctgcgcacgtccaacacggagccggtggtgcgtctgaacgttgagtccagagcggatactgcgttaatgaatgagaaaaccgccgagctgctcaacctgttaaaagaggaatcgctttga

SEQ ID NO: 34 - DNA, 786 bp, Klebsiella pneumoniae
  source 1..786: mol_type = genomic DNA
atgttttcagcgatctatcgctaccgtggctttattattgacagcgtcaaacgggactttcagtcccgttaccagactagcttcttaggcgcggcatggctgatcttacagccgatcgccatgatttccgtatatacattaatcttttctgagttaatgcgtgcccgcctggcgggcatggacggcccttttgcctacagtatctacctctgttccggggtgttaacctgggggctgtttacggaaacgctcggcaatctggtcaacgtttttctgaccaacgccaacattcttaaaaagcttagctttccgcggatctgtttaccgatcattgtcaccgcctcggcgttcattaacttcctgatcatttttggtctgtttgtactgtttctgatcgtcacgggcaatttcccgggcatgattttctttgaaatcattccggtgctgatcgttcagatgctgttcaccctcggcctcgggatcatcctcggggtgctgaacgtttttgtccgcgacgtcgggcagttcgtgaatatcctgctgcagttttggttctggtttacgcccattgtctacgtgtccaaaacgctgccggagtgggtctctggtctgctggcgtataacccgatggcgaccattatcggttcataccagaacgtgatgctctatcaccagagccctaactggctggcgctgcttccggtcacggtgctgtccgtcattctgtttttatttgcctggcgtttatttaaaaaacatgccgctgatattgtggacgagatttaa

SEQ ID NO: 35 - DNA, 1296 bp, Klebsiella pneumoniae
  source 1..1296: mol_type = genomic DNA
atgagtatcaaagttcagcacgtcggcaaggcgtataaatattatccctccaaatggaaccgggtcattgagaaacttctgccgggcgataagccgcggcacagcaagaaatgggtattgaaagatatcaatttcagtattgaacccggtgaagcggtcggcattgttggggtgaacggcgcaggtaaaagtacgttactgaagctgctgactggcaccactcagccgaccaaaggcagcattgagatccaggggcgtgtcgctgcgctgctggagctgggcatgggcttccatcctgactttaccggtcggcagaacgtgtatatgtccgggctgatgatgggcctgagccgggaagagattgagcgcttaatgccggagatcgaagcctttgcggatatcggtgactacattgaagagcccgtgcgcatctactccagcgggatgcaaatgcgcctggcgttcgccgtggccacggcctcacgcccggatattctgatcgtcgatgaagcgctttccgttggtgactcccgctttcaggcgaagtgctatgcccgtattgcggacttcaaaaagcagggcaccacgctgctgctggtctcccacagcgccggggatatcgtcaaacactgtgaccgcgccattttcctcaaaaatggtgatatctgtatggacggcaccgcccgtgacgtgaccaaccgttatctggatgagctgtttggcaaagccgacaaaaacagcgcgccaaaaagcgaaacggcaacctcgtcagccagcggcgaaagtcagatgtctctcgatgagattgaagatgtgtaccacacgcgcccaggctaccgtccggaagagtaccgttgggggcaggggggtgcaaaaatcattgattatcacatccaaagcgccggggttgattttccgccttcactgacgggcaatcagcagaccgatttcctgatgaaagtcgtatttgaatatgactttgattgcgtggtaccgggtttgttaatcaaaactctggatggcttatttctatatggtaccaactctttcctggcctcggaaggccgggaaaacatttcggtatcacgtggggacgttagagtatttaaattcagttttccggttgatttaaatagcggtgactatcttctgtcgtttggtatttcagagggaagcccgcaaaccgaaatgacgccgctcgatcgtcgctatgactccatcattttgcatgtaactaagagcatggatttctggggagtgattgacctgaagtcgactttcaatagttacaaatga

SEQ ID NO: 36 - DNA, 2058 bp, Klebsiella pneumoniae
  source 1..2058: mol_type = genomic DNA
atgactactaatacacataaattggttagcgaattacctgaaatttatcagactatttttgggcatcctgagtgggatggcgatgctgcacgagactgtaatgaacggctcgcgctaattagtgaacaatatgacagcttgtccagagagttaggaaggccactacgggttctcgacctgggctgtgctcaggggttcttcagtttaagtttggcaagcaagggtgccagcgtattaggtatcgactttttgcagcagaacattgatgtttgtcaggcgcttgctgaagaaaatccacattgtgatgttaaatttcaagtcgggcggatagaagacattgtcagcactctggaagaaaaccaatttgatctcgccattggactaagtgtttttcaccacattgttcatctgcatggggttgctgaagtcagatcgctgttagagcgtttggcaaatctgacgcaggcgatgattctcgagctcgctgtcaaggaggaaccactctattgggggaaatctcagcctgaagatccgcgtgaacttattgaccaatgtgctttctatcgattgattggaagatttgacactcatctgtctaatatttcacgtccgatgtatattatcagtaaccacagggttattcttccggaatttaatcagccttttacttcatggcgcgacagtccttacaccggagcaggctttgcgcataaacagagccgtcgctattatttctcttcggagttcatatgtaagttctatcgttttagtacagtaagttgcttactaactgataaggagagcgagcgtaatcgtactgaactcgcccatgaagaagcttttcttaaatctccaccatctggcttaaaagtgccggcgttgtttactgcaggggggaatggagaagcgggatggttggtaatggaaaaaattcccggagagctgttaaacgacgttctggccagtgaacggcatattgatcgggaaaaagttatttccgatctcctcgaccaattagttattttggaagaacatggtctatatcatgatgatttcagaacatggaatgttttaattgacgataatgacagcgctcgtttaatagattttggttcgattggcgatgtacaacaagactgcagctggccagttaatattttccagtcgttcattatttttgtaaatgaaatattttgtgaaaataaatcctggaggggcttctggcgttccgcaccattaagtcctttccagttgcctgaaccgtattcaaattggttgacagcattctggaaacatcctgttggtgagtggagttttgctttactccaacaactcttttcaaccaaagatgctctaccggctgcgagttccattatggacgcttctgatctatgggtccgggctcaggagcccgtattgttggaaagtcaaacgcaaatacgcaatacggatgcgcgggtagtccgtctcgagtcgcaaatcaatgaactcacctccctgattaatattatgggtgagagcattcagacgtttgagaagcgtgagtatccgccacaagacgttactactaatgtacagccgcgtatcgagattgagcagagtaaagccgttgattcagaagagattatgcgacttcatacgcagctcaatgatgctcagcaagaaatagagaatctacgtcatgagattgctaaaattcattatagtcgctcatggaaaatgaccaagtggtatcggtacgctggcttacagtactatctgcttcgtcagtacggcttcaaacagcgttttaagcatttactcaaacgagtgcttagcaacgtaatttattttttgcgtgcacatccacgactaaagcagaaggtgatcaatctactgcgtacaattggaatttatgactttgcttatcgtatgcatcgtcgtatgaatcctggttcacataacccttatccaaacgacccacaataccagtcgcagactgaaaagcagatcttacatccagagttattgcctccggaagttaactcaatttttagcgagcttaaaaacaaaagataa

SEQ ID NO: 37 - DNA, 2523 bp, Klebsiella pneumoniae
  source 1..2523: mol_type = genomic DNA
ttgcatattttgattgacgtacaaggatatcaatcggaaagtaaattccgtggagttggtcgcagcacctatgaaatgagtcgtgcgatcataaaaaatgctggccagcatcgagtaagcattttaatgaatggcatgtattcgattgatagtataaatgaaattaaaaaaagctggggtgatatattaccgcaggaagaaatgtttattttttcagctgctggccctacagctcttcgcgactgtgaaaaccatccccggagtgttgccgccacactagctcgtgaacttgctattgctaatatcaatcccgacgttgtttttattattaatttctacgaaggttttgacgatagttataccgtctcaattcctcaaactacagtaccatggaaaacagtttgtgtttgtcacgatctaattccgttactgaataaagaacgctatctgggcgaaccaaacttccgtcagtattattatgataaactagctcaatacgaaagggcggacgctatttttgctatttccagatcatccatgcaggaagttatcgattacacatcgattccggcagaaaaaattattaatatttcatctggagtaagcgattcatttaaaattaaagattatactcacgatgaaatcaaagacttacgtaataaatatcatcttcctcaagagtttattctttctttggcaatgatagagccacgtaaaaatattgaagcgctgattcatgcatatagtttattaccgcatgccctgcaacagagttatcccttagttttagcctataaaattagcaccgatgaaaaggaaaggctgtaccgagttgcagagaactatggtttatctcgtaatcagcttatttttacaggcttcttaaacgatagtgaccttatcgcactttacaatttgtgcaaaattttcgttttcccctctatacatgaagggtttggcctgccgccactagaagctatgcgttgtggtgcagctacgctgggttcaaatgtgaccagcttacccgaggtcatcggtatggaagaggctttatttaatcctctggatgtccccgacatttgccgtgttatgcaaagggccttgactgacagtgagttctactcagcattaaaagctcatgctccggcgcaggcggcaaagttcacatgggatcacaccgcgcagctcgcgttaaagggatttgagaggcttgtagataaggcttccgcatcagaacctctggatatcacaagcttcaccgcatacaccattaatagaattaaaaatattgcagaattaagtgaaaccgaacgcttacagacagcctgggcgattgctcgtaatagctttgctacacatcagcgcaagctgctggttgatatttctgttcttgttgagcatgatgcgaaaacgggaattcaacgggtttctcgcagtatacttagtgaattactgaaatctggcgttgctggttatactgtcagtgcggtttattatcgaccgggtgaatgctatcgctatgccaacgaatacctgaatacccattttaacggggcgttcgggcctgatgtacctgtactgtttaccaaagatgatattctggttgctaccgatctaactgcccatctgtttcctgagcttactgtccagctggattttattcgtctatccggtgccaaggtttgttttgttgtgcatgacattttgcctctgagaagaccggagtggagcgatgagggaatgcaacgcgtgttccccatttggttatcttgcattgcgcagcacgcagaccgcttgatttgtgtatcagcaagcgttgcagaggatgtaaaagcctggattgcggaaaacagccattgggtgaaaccgaacccgctgctgaccgtcagcaacttccatctgggagccgacctcgatgccagcgtaccgtccactggcatgccggataatgcccaggcgctgttagcagcgatggccgcggctccatcatttatcatggtgggcacgatggaaccacgcaaaggacatgcgcagacgctagcggcatttgaagaattgtggttacagggcaagaactacaatctgtttatcattggtaaacaggggtggcatgttgatgatttatgtgaacgtttacgtcaccatccacagctaaataaaaaactattttggctacaaaacattagcgatgagttccttacgaagttgtattctcagtctagtgcgttaatcttcgcatctctcggagaaggctttggcctgccgttgattgaagcggcgcagaaaaagctgccggtgattatccgtgacattccggtgtttaaagagattgctcaggaacatgcgtggtatttctccggggaagcgccggccgacatcgcgaaggccgtcgaagactggttagccctgtatgagcaaaacgcgcatcctcgttccgagaatatcaactggttaacctggaagcagagcgcggaatttctcctgaaaaacctgccgattatcgcgccagccgcgaagcaataa

SEQ ID NO: 38 - DNA, 1146 bp, Klebsiella pneumoniae
  source 1..1146: mol_type = genomic DNA
atgaaaattatttttgctactgagccaattaaatacccgttaacgggcatcggtcggtattccctggagctggttaagcggctggcggtcgcccgcgaaatcgaagagctgaagctgtttcacggcgcgtcgtttatcgatcagatcccccaggtggagaataaaagcgataccaaagccagcaatcatggtcgtttgtcggcgtttctgcgccgccagccgctgctgattgaggcgtatcgcctgctgcacccgcggcgccaggcgtgggcattgcgcgactataaagattatatctaccatggtcccaatttttacctgccgcatcgcctggaacacgccgtgaccacgtttcatgacatctccatttttacctgcccggaatatcatccaaaagatcgggttcgctatatggagaagtccctgcatgagagcctggattcggcaaagctgatcctgaccgtctctgacttctcgcgcagtgaaatcatccgcctgttcaactatccggcggagcggatcgtcaccaccaagctggcctgcagcagcgactatattccacgcagcccggcggagtgcctgccggtcctgcagaaatatcagctggcgtggcaggggtatgcgttatatatcggcaccatggagccgcgtaaaaatatccgtggtctgctgcaggcctatcagctgctgccgatggagacccgcatgcgctacccgctgatcctcagcggctatcgcggctgggaagacgatgtgctgtggcagttagtcgagcgtggtacgcgtgaagggtggatccgttacctgggctatgtcccggatgaggacctgccttatctgtacgcggcggccagaacctttgtttatccctccttctatgagggattcggtttacctattcttgaagcgatgtcttgcggtgtgccggtagtatgttccaatgtcacttctttgcctgaggtggttggcgatgccggcctcgttgccgatcctaatgatgtagacgcgattagcgcgcatattttgcagagcctgcaggatgatagctggcgggaaatcgccaccgcgcgcggtcttgcccaggcgaaacagttttcgtgggagaactgtacgacccagaccattaacgcctataaattactctaa

SEQ ID NO: 39 - DNA, 1116 bp, Klebsiella pneumoniae
  source 1..1116: mol_type = genomic DNA
ttgagagttctacacgtctataagacctactatcccgatacctacggcggtattgagcaggtcatttatcagctcagtcagggttgcgcccgccgggggatcgcagccgatgtttttacttttagcccggacaaagagacaggtcctgtcgcctacgaagaccatcgggtcatttataataagcagctttttgaaattgcctccacgccgttttcgttgaaagcgttaaagcgttttaagcagattaaagatgattacgacatcatcaactaccattttccgtttccctttatggatatgctgcatctctcggcgcggcctgacgccagaacggtggtgacctatcactcggatattgtgaaacaaaaacggttgatgaagttgtaccagccgctgcaggagcgattcctcgccagcgtagactgcattgttgcctcgtcgcccaactacgtggcctccagccagaccctgaaaaaatatcaggataaaacggtggtgatcccgtttggtctggagcagcatgacgtgcagcacgatccgcagcgggtggcgcactggcgggaaaccgtcggcgataacttcttcctcttcgtcggcgctttccgctactacaaagggctgcacattctgctggatgccgccgagcgtagccggctgccagtggtgatcgtcgggggcgggccgctggaggcggaggtgcggcgtgaggcgcagcagcgcggactgagcaatgtggtgtttaccggcatgctcaacgacgaagataaatacattctcttccagctctgccggggcgtggtcttcccctcgcatctgcgctcagaggcgtttggcattacgttactggaaggcgcgcgctttgccaggccgctgatctcctgcgagatcggcaccggtacctcgttcattaaccaggacaaagtaaatggctgcgtgatcccgccgaatgacagtcaggcgctggtggaggcgatgaatgagctctggcataacgatgaaaccgccagccgctatggcgaaaactcgcgtcgtcgttttgaagagatgtttacagccgaccatatgattgacgcttacgtcaatctctacactacgctgctggaaagcaaatcctga

SEQ ID NO: 40 - DNA, 27 bp, synthetic construct
  misc_feature 1..27: note = PCR Primer
  source 1..27: mol_type = other DNA
gaaggcgggcgcgtgaccattctcggc

SEQ ID NO: 41 - DNA, 27 bp, synthetic construct
  misc_feature 1..27: note = PCR Primer
  source 1..27: mol_type = other DNA
gccgagaatggtcacgcgcccgccttc

(19) United States
(12) Patent Application Publication
(10) Pub. No.: US 2026/0240990 A1
(43) Pub. Date: Aug. 20, 2026

(54) Immunogenic Composition

(71) Applicant: GlaxoSmithKline Biologicals SA, Rixensart (BE)

(72) Inventors: Cristina ALAIMO, Schlieren (CH); Maria Paula CARRANZA SANDMEIER, Schlieren (CH); Michael Thomas KOWARIK, Schlieren (CH); Patricia MARTIN KILLIAS, Schlieren (CH)

(73) Assignee: GlaxoSmithKline Biologicals SA, Rixensart (BE)

(21) Appl. No.: 19/156,991

(22) PCT Filed: Feb. 21, 2024
(86) PCT No.: PCT/EP2024/054344

§ 371 Date: Aug. 15, 2025

(30) Foreign Application Priority Data
Feb. 23, 2023 (GB) 2302579.4

(51) Int. Cl. A61K 39/385; A61K 39/00; A61K 39/108

(52) U.S. Cl. CPC A61K 39/385; A61K 39/0266; A61K 2039/545; A61K 2039/55; A61K 2039/6087

(57) ABSTRACT

The present invention relates to the field of immunogenic compositions and vaccines, their manufacture, host cells which can be used in their manufacture and the use of such immunogenic compositions and vaccines in medicine. More particularly, it relates to an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a carrier protein (e.g. a detoxified Exotoxin A of Pseudomonas aeruginosa (EPA)) wherein the Klebsiella pneumoniae O3b O-antigen polysaccharide conjugate is present in said immunogenic composition at a higher saccharide dose than at least one of the Klebsiella pneumoniae O1v1 O-antigen polysaccharide conjugate, Klebsiella pneumoniae O2a O-antigen polysaccharide conjugate or Klebsiella pneumoniae O2afg O-antigen polysaccharide conjugate.

### FIELD OF THE INVENTION

The present invention relates to the field of immunogenic compositions and vaccines, their manufacture and the use of such immunogenic compositions and vaccines in medicine. More particularly, it relates to immunogenic compositions comprising Klebsiella pneumoniae O-antigen polysaccharide conjugates.

### BACKGROUND TO THE INVENTION

Klebsiella pneumoniae is a gram-negative, encapsulated non-motile bacteria of the Enterobacteraceae family. It colonizes the gastrointestinal, respiratory and urinary tracts and is carried asymptomatically as part of the human microbiome. Klebsiella pneumoniae is an important cause of community, long term care facilities and hospital-acquired infections. It is among leading causes of serious infections in newborns, blood cancer patients, and other immunocompromised patients. It causes: urinary tract infections, pneumonia, bacteraemia and soft tissue infections. Infections caused by Klebsiella pneumoniae are responsible for high rates of morbidity and mortality. The mortality rate of Klebsiella bacteraemia and pneumonia can exceed 50% even with antimicrobial therapy. In K. pneumoniae, carbapenemases are the main contributing factor to extensive drug resistance (David et al. (2019) Nature Microbiology, VOL 4, 1919-1929). The emergence of hypervirulent isolates and the increase in isolates resistant to β-lactams, including carbapenems, and limited treatment options make Klebsiella pneumoniae a global health concern. Alternative approaches to antibiotics are highly needed (HyperTextTransferProtocolSecure: //www.who.int/medicines/publications/global-priority-list-antibiotic-resistant-bacteria/en). However, there is currently no vaccine on the market.

Klebsiella pneumoniae expresses two types of polysaccharide molecules on the surface: capsular polysaccharide (K-antigen) and lipopolysaccharide (O-antigen, also known as O-antigen polysaccharides or OPS). Capsule polysaccharides are highly diverse with at least 77 serologically distinct K-antigens. In contrast, the diversity of O-antigen structures in the lipopolysaccharides of Klebsiella pneumonia is limited. Nine serotypes have been identified: O1, O2, O2ac, O3, O4, O5, O7, O8, and O12. There are subtypes within these serogroups, for example, O3 serogroup has three different subtypes differing in the number of mannose residues within the O-antigen repeating units (Guachalla et al. (2017) Scientific Reports 7:6635, 1-13). The carbohydrate repeating unit structures of OPSs of K. pneumoniae are described in FIG. 1 of Clarke et al. J. Biol. Chem. (2018) 293 (13) 4666-4679 and FIG. 1 of Kelly et al. J. Biol. Chem. (2019) 294 (28) 10863-10876, which also describe the biosynthesis of certain O-antigens. According to Clarke et al. (2018) genes outside the main rfb (O-antigen biosynthesis) locus (i.e. the six genes wzm-wbbO) can have profound effects on the final structure (see FIG. 2 of Clarke et al.).

Conjugate vaccines (vaccines comprising a carrier protein covalently linked to an immunogenic antigen) have been a successful approach for vaccination against a variety of bacterial infections. Conjugation of T-independent antigens, for example saccharides, to carrier proteins has long been established as a way of enabling T-cell help to become part of the immune response for a normally T-independent antigen. In this way, an immune response can be enhanced by allowing the development of immune memory and boostability of the response. Hegerle et al. (2018) (PLoS ONE 13 (9): e0203143) report the development of a combined Klebsiella pneumoniae and Pseudomonas aeroginosa glycoconjugate vaccine comprised of the four most common Klebsiella pneumoniae OPS types associated with human infections (01, 02, 03, 05), chemically linked to the two flagellin types of Pseudomonas aeruginosa (FlaA, FlaB).

There is a need to develop vaccines which can protect against Klebsiella pneumoniae infections. In particular, there is a need for a broad spectrum vaccine.

### SUMMARY OF THE INVENTION

The present invention provides immunogenic compositions (e.g. vaccines) and methods of using them to protect against Klebsiella pneumoniae infections, in particular, protect against a specific combination of subserotypes of Klebsiella pneumoniae. These immunogenic compositions and methods consider the prevelance of certain Klebsiella pneumoniae subserotypes (i.e., O1v1 vs O1v2, O2afg vs O2a, O3 vs O3b), consider antibiotic resistant Klebsiella pneumoniae, and consider cross-reactivities between distinct Klebsiella pneumoniae subserotypes. These subserotypes (in particular the prevelance of subserotypes in patients infected by Klebsiella pneumoniae) are useful for protecting against Klebsiella pneumoniae infections. Immunogenic compositions and vaccines of the present invention provide broad coverage against several different subserotypes of Klebsiella pneumoniae. Furthermore, the present invention also provides the first information on the safety and immunogenicity of conjugates, in particular bioconjugates, against the subserotypes O1v1, O2a, O2afg, O3b of Klebsiella pneumoniae in a clinical trail. The relative immunogenicities of the different serotypes informs the doses of the different serotypes which can be used in the immunogenic compositions (e.g. vaccines) and methods of the invention.

Accordingly, there is provided in one aspect of the present invention, an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a carrier protein (e.g. a detoxified Exotoxin A of Pseudomonas aeruginosa (EPA)) wherein the Klebsiella pneumoniae O3b O-antigen polysaccharide conjugate is present in said immunogenic composition at a higher saccharide dose than at least one of the Klebsiella pneumoniae O1v1 O-antigen polysaccharide conjugate, Klebsiella pneumoniae O2a O-antigen polysaccharide conjugate or Klebsiella pneumoniae O2afg O-antigen polysaccharide conjugate.

According to a further aspect of the invention, there is provided a process for making an immunogenic composition of the invention, comprising combining a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, and optionally a pharmaceutically acceptable excipient and/or carrier.

According to a further aspect of the invention, there is provided a host cell comprising:
i) nucleotide sequences comprising polysaccharide synthesis genes for producing a Klebsiella pneumoniae O-antigen polysaccharide selected from O1v1, O2a, O2afg and O3b, optionally integrated into the host cell genome;
ii) a nucleotide sequence encoding a heterologous oligosaccharyl transferase, optionally within a plasmid;
iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and
iv) a nucleotide sequence encoding an ABC transporter, optionally K. pneumoniae genes wzm and wzt, optionally integrated into the host cell genome.

According to a further aspect of the invention, there is provided a process for making an immunogenic composition of the invention, comprising combining a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate such that the Klebsiella pneumoniae O3b O-antigen polysaccharide conjugate is present in said immunogenic composition at a higher saccharide dose than at least one of the Klebsiella pneumoniae O1v1 O-antigen polysaccharide conjugate, Klebsiella pneumoniae O2a O-antigen polysaccharide conjugate or Klebsiella pneumoniae O2afg O-antigen polysaccharide conjugate, and optionally adding a pharmaceutically acceptable excipient and/or carrier.

According to a further aspect of the invention, there is provided an immunogenic composition comprising the conjugate (e.g. bioconjugate) of the invention, and optionally a pharmaceutically acceptable excipient and/or carrier.

According to a further aspect of the invention, there is provided a vaccine comprising the immunogenic composition of the invention and optionally an adjuvant.

According to a further aspect of the invention, there is provided a method of inducing an immune response to Klebsiella pneumoniae in a subject, the method comprising administering a therapeutically or prophylactically effective amount of the immunogenic composition of the invention, or the vaccine of the invention, to a subject in need thereof.

According to a further aspect of the invention, there is provided an immunogenic composition of the invention, or the vaccine of the invention, for use in inducing an immune response to Klebsiella pneumoniae in a subject.

According to a further aspect of the invention, there is provided an immunogenic composition of the invention for use in the manufacture of a medicament for inducing an immune response to Klebsiella pneumoniae in a subject.

### DESCRIPTION OF DRAWINGS/FIGURES

FIGS. 1A and 1B: Analysis of the O3b and O2afg glycan-producing strains (A and B, respectively) when transformed with plasmids encoding pglB and EPA with different number of PglB glycosylation consensus sequences. Periplasmic extracts were used for O3b (A), while enriched periplasmic extracts were used for O2afg (B). The used carriers contain 3 glycosylation sites (B, lane 1), 4 glycosylation sites (A, lane 7; B lane 2), 5 glycosylation sites (A, lanes 1, 2, and 3), 6 glycosylation sites (A, lanes 4 and 5), 7 glycosylation sites (A, lane 6). PAGERULER™ Prestained Protein Ladder (ThermoFisher) is indicated by “M”.

FIGS. 2A, 2B, 2C, and 2D: Analysis of the O1v1, O2a, O2afg, and O3b-conjugate-producing strains' products (A, B, C, and D, respectively). Two experimental replicates per serotype are analysed. Coomassie staining (A, left picture; B; C, right picture; D), anti K. pneumoniae O1v1 Western blot (A, central picture), anti K. pneumoniae O2a Western blot (A, right picture; C, left picture), anti K. pneumoniae O2afg Western blot (C, central picture) are shown. PAGERULER™ Prestained Protein Ladder (ThermoFisher) is loaded in lanes 1, 4, 9, 10, the corresponding band size in kDa is reported. Other lanes contain the two replicas from each conjugate-producing strain.

FIG. 3 Purified conjugates were analyzed via SDS-PAGE and Coomassie staining.

FIG. 4 IgG titers analysed in sera of rabbits immunized with 1 μg polysaccharide of polyvalent conjugate compostition. Only Pre-immunization and Post-III sera results are reported. Lines and bars indicate the geometric mean titer (GMT)+/−95% confidence interval. ****: p<0.0001, **: p<0.01, ANOVA-Sidak's multiple comparisons. “Control” indicates immunizations carried out with buffer only.

FIG. 5 O2a opsonisation index (OI) in pre- and post-III immunization sera from rabbit immunized with monovalent O2a conjugate or polyvalend Kp5v composition. O2a wild type strain was used. Control group are animals immunized with buffer alone. Lines and bars indicate the GMT+/−95% confidence interval. ****: p<0.0001, ***: p<0.001, **: p<0.01, ANOVA-Sidak's multiple comparisons.

FIG. 6 K. pneumoniae wild type strains were tested for binding with pools of sera of animals immunized with monovalent vaccine via flow cytometry. Median fluorescence intensity due to the binding of the antisera to the cells is reported. Mean and standard deviaton are shown. New Zealand white rabbits were injected at days 0, 14 and 28 with 1 μg of monovalent vaccine with no adjuvant. Control group are animals immunized with buffer alone.

FIG. 7A and FIG. 7B Two different rat monoclonal antibodies raised against KpO1v1 (Z+) were tested in ELISA experiments in which plates were coated with KpO1v1 (Z+)-EPA, KpO1v1 (Z−)-EPA and KpO1v2. mAB #563 specifically recognizes glycans produced from a strain containing wbbZ (KpO1v1 (Z+) or KpO1v2, A). mAB 1B3B3-1 does not discriminate the different conjugates (B).

FIG. 8 IgG concentrations (LIA AU) in pre- and post-III immunization with monovalent pyruvylated-(Z+) and non-pyruvylated-(Z−) O1v1-EPA rabbit sera. Lines indicate the GMC +/−95% confidence interval. ****: p<0.0001, ***: p<0.001 One-way ANOVA. The text above the graphs identifies the tested LPS, while immunization groups are described below the graphs.

FIG. 9 IgG concentrations (LIA AU) in pre- and post-III Kleb4V+/− AS03 immunization rabbit sera. Lines indicate the GMC +/−95% confidence interval. ****: p<0.0001, ***: p<0.001 One-way ANOVA. The text above the graphs identifies the tested LPS, while immunization groups are described below the graphs.

FIG. 10 Kleb4v vaccine elicits a robust immune response in a Phase I/II clinical trial. Graph showing the % of responders by Luminex binding assay following one (first, light shaded column) or 2 (second, dark shaded column) immunisations with the Kleb4V vaccine, either unadjuvenated or adjuvented with an ASO3 adjuvant, and at a low dose (16 and 16 A, respectively) or at a target dose (64 and 64 A, respectively), compared with placebo (P).

FIGS. 11A, 11B and 11C Functional immune response of sera from Phase I/II clinical trial. FIG. 11A shows SBA responses to O2afg, FIG. 11B shows OPA responses to O1v1 and FIG. 11C shows OPA responses to O1v2. The lines marked with circles show the placebo response (P), the lines marked with squares (16) show the response for a total saccharide dose of 16 μg (4 μg/serotype), the lines marked with triangles (16 A) show the responses for a total saccharide dose of 16 μg with adjuvant, the lines marked with inverted triangles (64) show the responses for a total saccharide dose of 64 μg (16 μg/serotype) and the lines marked with diamonds (64 A) show the responses for a total saccharide dose of 64 μg with adjuvant.

### DETAILED DESCRIPTION OF THE INVENTION

### Definitions

Carrier protein: a protein which may be covalently attached to an antigen (e.g. saccharide antigen, such as a bacterial polysaccharide antigen) to create a conjugate (e.g. bioconjugate). A carrier protein activates T-cell mediated immunity in relation to the antigen to which it is conjugated.

EPA: Exotoxin A of Pseudomonas aeruginosa (also known as “Exotoxin of P. aeruginosa”, “EPA”, or “ETA”)

Any amino acid except proline (pro, P): refers to an amino acid selected from the group consisting of alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), serine (ser, S), threonine (thr, T), tryptophan (trp, W), tyrosine (tyr, Y), and valine (val, V).

Naturally occurring amino acid residues: amino acids that are naturally incorporated into polypeptides. In particular, the 20 amino acids encoded by the universal genetic code: alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

O-Antigens (also known as O-specific polysaccharides or O-side chains): a component of the surface lipopolysaccharide (LPS) of Gram-negative bacteria. Examples include O-antigens from Klebsiella pneumoniae. As used herein a “Klebsiella pneumoniae O-antigen polysaccharide O1v1” is an O-antigen polysaccharide from Klebsiella pneumoniae serotype O1v1. As used herein a “Klebsiella pneumoniae O-antigen polysaccharide O2a” is an O-antigen polysaccharide from Klebsiella pneumoniae serotype O2a. As used herein a “Klebsiella pneumoniae O-antigen polysaccharide O2afg” is an O-antigen polysaccharide from Klebsiella pneumoniae serotype O2afg. As used herein a “Klebsiella pneumoniae O-antigen polysaccharide O3b” is an O-antigen polysaccharide from Klebsiella pneumoniae serotype O3b.

Lipopolysaccharide (LPS): large molecules consisting of a lipid and a polysaccharide composed joined by a covalent bond.

wzy: a polysaccharide polymerase gene encoding an enzyme which catalyzes polysaccharide polymerization. The encoded enzyme transfers oligosaccharide units to the non-reducing end forming a glycosidic bond.

waal: a O-antigen ligase gene encoding a membrane bound enzyme. The encoded enzyme transfers undecaprenyl-diphosphate (UPP)-bound O-antigen to the lipid A core oligosaccharide, forming lipopolysaccharide.

“D-galactan I” as used herein is a reference to a polymer built of [→3)-β-D-Galf-(1→3)-α-D-Galp-(1→] repeating units (see Hsieh et al. 2014 Front. Microbiol. 5:608, doi: 10.3389/fmicb.2014.00608).

“D-galactan II” as used herein is a reference to a polymer built of [→3)-α-D-Galp-(1→3)-β-D-Galp-(1→] repeating units (see Hsieh et al. 2014 Front. Microbiol. 5:608, doi: 10.3389/fmicb.2014.00608).

“D-galactan III” as used herein is a reference to a polymer built of [→3)-β-D-Galf-(1→3)-[α-D-Galp-(1→4)]-α-D-Galp-(1→] repeating units (see Stojkovic et al. 2017 Front. Microbiol. 8:684, doi: 10.3389/fmicb.2017.00684).

“GlcNAc” as used herein is a reference to N-Acetylglucosamine.

“Gal” or “Galp” as used herein is a reference to D-galactopyranose.

“Galf” as used herein is a reference to D-galactofuranose.

“Man” as used herein is a reference to D-Mannopyranose.

As used herein, the term “conjugate” refers to carrier protein covalently linked to an antigen. For example, a Klebsiella pneumoniae O1v1 O-antigen polysaccharide conjugate comprises a carrier protein covalently linked to an Klebsiella pneumoniae O1v1 O-antigen polysaccharide. For example, a Klebsiella pneumoniae O2a O-antigen polysaccharide conjugate comprises a carrier protein covalently linked to an Klebsiella pneumoniae O2a O-antigen polysaccharide. For example, a Klebsiella pneumoniae O2afg O-antigen polysaccharide conjugate comprises a carrier protein covalently linked to an Klebsiella pneumoniae O2afg O-antigen polysaccharide. For example, a Klebsiella pneumoniae O3b O-antigen polysaccharide conjugate comprises a carrier protein covalently linked to an Klebsiella pneumoniae O3b O-antigen polysaccharide.

As used herein, the term “bioconjugate” refers to conjugate between a protein (e.g. a carrier protein) and an antigen (e.g. a saccharide antigen, such as a bacterial polysaccharide antigen) prepared in a host cell background, wherein host cell machinery links the antigen to the protein (e.g. N-linked glycosylation).

As used herein an amino acid sequence may have a certain % identity to a reference amino acid sequence. Variants may differ from the reference amino acid sequence by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, 1 to 10, 5 to 10, 1 to 5, 1 to 3, 1 to 2 or 1 amino acids of the reference amino acid sequence may be substituted or deleted.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the terms “insertion” or “addition” (including other tenses thereof such as “inserted”) means the addition of one or more non-native amino acid residues in the protein sequence or, as the context requires, addition of one or more non-native nucleotides in the polynucleotide sequence. Typically, no more than about from 1 to 10 residues, (e.g. 1 to 7 residues, 1 to 6 residues, or 1 to 4 residues) are inserted at any one site within the protein molecule.

As used herein, the term “added next to” is the addition of one or more non-native amino acid residues in the protein sequence at a position adjacent to the referenced amino acid or amino acid region.

A “consensus sequence” is a sequence have a specific structure and/or function. As used herein, the term “consensus sequence” is a sequence comprising a glycosite. A consensus sequence may be selected from: a five amino acid consensus sequence D/E-X-N-Z-S/T (SEQ ID NO: 1), a seven amino acid consensus sequence K-D/E-X-N-Z-S/T-K (SEQ ID NO: 2) or an extended consensus sequence (e.g. J-D/E-X-N-Z-S/T-U (SEQ ID NO: 4)).

Unless specifically stated otherwise, providing a numeric range (e.g. “25-30”) is inclusive of endpoints (i.e. includes the values 25 and 30).

The terms “identical” or percent “identity” refer to nucleotide sequences or amino acid sequences that are the same or have a specified percentage of nucleotide residues or amino acid residues that are the same (e.g. 80%, 85%, 90%, 92%, 95%, 96%, 97%, 98% or 99% identity over a specified region), when compared and aligned for maximum correspondence using, for example, sequence comparison algorithms or by manual alignment and visual inspection. Identity between polypeptides may be calculated by various algorithms. In general, when calculating percentage identity the two sequences to be compared are aligned to give a maximum correlation between the sequences. This may include inserting “gaps” in either one or both sequences, to enhance the degree of alignment. For example the Needleman Wunsch algorithm (Needleman and Wunsch 1970, J. Mol. Biol. 48:443-453) for global alignment, or the Smith Waterman algorithm (Smith and Waterman 1981, J. Mol. Biol. 147:195-197) for local alignment may be used, e.g. using the default parameters (Smith Waterman uses BLOSUM 62 scoring matrix with a Gap opening penalty of 10 and a Gap extension penalty of 1). A preferred algorithm is described by Dufresne et al. in Nature Biotechnology in 2002 (vol. 20, pp. 1269-71) and is used in the software GenePAST (Genome Quest Life Sciences, Inc. Boston, MA). The GenePAST “percent identity” algorithm finds the best fit between the query sequence and the subject sequence, and expresses the alignment as an exact percentage. GenePAST makes no alignment scoring adjustments based on considerations of biological relevance between query and subject sequences. Identity between two sequences is calculated across the entire length of both sequences and is expressed as a percentage of the reference sequence (e.g. SEQ ID NO: 16 of the present invention).

As used herein the term “recombinant” means artificial or synthetic. In an embodiment, a “recombinant protein” refers to a protein that has been made using recombinant nucleotide sequences (nucleotide sequences introduced into a host cell). In an embodiment, the nucleotide sequence that encodes a “recombinant protein” is heterologous to the host cell.

As used herein the terms “isolated” or “purified” mean a protein, conjugate (e.g. bioconjugate), polynucleotide, or vector in a form not found in nature. This includes, for example, a a protein, conjugate (e.g. bioconjugate), polynucleotide, or vector having been separated from host cell or organism (including crude extracts) or otherwise removed from its natural environment. In an embodiments, an isolated or purified protein is a protein essentially free from all other polypeptides with which the protein is innately associated (or innately in contact with).

As used herein, the term “subject” refers to an animal, in particular a mammal such as a primate (e.g. human).

As used herein, the term “effective amount,” in the context of administering a therapy (e.g. an immunogenic composition or vaccine of the invention) to a subject refers to the amount of a therapy which has a prophylactic and/or therapeutic effect(s). In an embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

As used herein, a “multivalent immunogenic composition” or “multivalent vaccine” is an immunogenic composition/vaccine that comprises two or more different antigens. In a particular embodiment, the multivalent immunogenic composition/vaccine comprises two or more different serotypes or subserotypes of a particular pathogen (e.g. against two or more different subserotypes of Klebsiella pneumoniae).

The term “comprises” is open-ended and means “includes.” Thus, unless the context requires otherwise, the word “comprises” or “has”, and variations thereof (including “comprise” and “comprising” or “have” and “having”, respectively), will be understood to imply the inclusion of a stated compound(s), molecule(s), composition(s), or steps, but not to the exclusion of any other compound(s), molecule(s), composition(s), or steps. The terms “comprising” and “having” when used as a transition phrase herein are open-ended whereas the term “consisting of” when used as a transition phrase herein is closed (i.e., limited to that which is listed and nothing more). In an embodiments and for readability, the word “is” may be used as a substitute for “consists of” or “consisting of”. The abbreviation, “e.g.” is derived from the Latin exempli gratia, and is used herein to indicate a non-limiting example. Thus, the abbreviation “e.g.” is synonymous with the term “for example”.

### Immunogenic Compositions

The present invention provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate. Each of the Klebsiella pneumoniae O1v1, O2a, O2afg and O3b O-antigen polysaccharides are individually conjugated to a carrier protein (e.g. a detoxified Exotoxin A of Pseudomonas aeruginosa (EPA)) and the Klebsiella pneumoniae O3b O-antigen polysaccharide conjugate is present in said immunogenic composition at a higher saccharide dose than at least one of the Klebsiella pneumoniae O1v1 O-antigen polysaccharide conjugate, Klebsiella pneumoniae O2a O-antigen polysaccharide conjugate or Klebsiella pneumoniae O2afg O-antigen polysaccharide conjugate.

In an embodiment, the Klebsiella pneumoniae O3b O-antigen polysaccharide conjugate is present at a higher saccharide dose than the Klebsiella pneumoniae O1v1 O-antigen polysaccharide conjugate. In an embodiment, the Klebsiella pneumoniae O3b O-antigen polysaccharide conjugate is present at a higher saccharide dose than the Klebsiella pneumoniae O2a O-antigen polysaccharide conjugate. In an embodiment, the Klebsiella pneumoniae O3b O-antigen polysaccharide conjugate is present at a higher saccharide dose than the Klebsiella pneumoniae O2afg O-antigen polysaccharide conjugate. In an embodiment, the Klebsiella pneumoniae O3b O-antigen polysaccharide conjugate is present at a higher saccharide dose than each of the Klebsiella pneumoniae O1v1 O-antigen polysaccharide conjugate, Klebsiella pneumoniae O2a O-antigen polysaccharide conjugate and Klebsiella pneumoniae O2afg O-antigen polysaccharide conjugate. In an embodiment, the saccharide dose of the Klebsiella pneumoniae O3b O-antigen polysaccharide conjugate is at least 1.5, 2, 3, 4, 5, 6, 7, 8, 9 or 10 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is at least 2 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is at least 4 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is at least 5 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is at least 10 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is at least 1.5, 2, 3, 4, 5, 6, 7, 8, 9 or 10 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is at least 2 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is at least 4 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is at least 5 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is at least 10 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is at least 1.5, 2, 3, 4, 5, 6, 7, 8, 9 or 10 times higher than the saccharide dose of the Klebsiella pneumoniae O2afg O-antigen polysaccharide conjugate. In an embodiment, the saccharide dose of the Klebsiella pneumoniae O3b O-antigen polysaccharide conjugate is at least 2 times higher than the saccharide dose of the Klebsiella pneumoniae O2afg O-antigen polysaccharide conjugate. In an embodiment, the saccharide dose of the Klebsiella pneumoniae O3b O-antigen polysaccharide conjugate is at least 4 times higher than the saccharide dose of the Klebsiella pneumoniae O2afg O-antigen polysaccharide conjugate. In an embodiment, the saccharide dose of the Klebsiella pneumoniae O3b O-antigen polysaccharide conjugate is at least 5 times higher than the saccharide dose of the Klebsiella pneumoniae O2afg O-antigen polysaccharide conjugate. In an embodiment, the saccharide dose of the Klebsiella pneumoniae O3b O-antigen polysaccharide conjugate is at least 10 times higher than the saccharide dose of the Klebsiella pneumoniae O2afg O-antigen polysaccharide conjugate. In an embodiment, the saccharide dose of the Klebsiella pneumoniae O3b O-antigen polysaccharide conjugate is at least 1.5, 2, 3, 4, 5, 6, 7, 8, 9 or 10 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate. In an embodiment, the saccharide dose of the Klebsiella pneumoniae O3b O-antigen polysaccharide conjugate is at least 2 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate. In an embodiment, the saccharide dose of the Klebsiella pneumoniae O3b O-antigen polysaccharide conjugate is at least 4 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate. In an embodiment, the saccharide dose of the Klebsiella pneumoniae O3b O-antigen polysaccharide conjugate is at least 5 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate. In an embodiment, the saccharide dose of the Klebsiella pneumoniae O3b O-antigen polysaccharide conjugate is at least 10 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate.

In an embodiment, the saccharide dose of the Klebsiella pneumoniae O3b O-antigen polysaccharide conjugate is between 1.5 and 20 or 2 and 10 or 2 and 5 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is between 2 and 5 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is between 1.5 and 20 or 2 and 10 or 2 and 5 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is between 2 and 5 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is between 1.5 and 20 or 2 and 10 or 2 and 5 times higher than the saccharide dose of the Klebsiella pneumoniae O2agf O-antigen polysaccharide conjugate. In an embodiment, the saccharide dose of the Klebsiella pneumoniae O3b O-antigen polysaccharide conjugate is between 2 and 5 times higher than the saccharide dose of the Klebsiella pneumoniae O2agf O-antigen polysaccharide conjugate. In an embodiment, the saccharide dose of the Klebsiella pneumoniae O3b O-antigen polysaccharide conjugate is between 1.5 and 20 or 2 and 10 or 2 and 5 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate. In an embodiment, the saccharide dose of the Klebsiella pneumoniae O3b O-antigen polysaccharide conjugate is between 2 and 5 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate. In an embodiment, the saccharide dose of the Klebsiella pneumoniae O3b O-antigen polysaccharide conjugate is between 2 and 10 or 2 and 10 or 2 and 5 times higher than the saccharide dose of the Klebsiella pneumoniae O1v1 O-antigen polysaccharide conjugate. In an embodiment, the saccharide dose of the Klebsiella pneumoniae O3b O-antigen polysaccharide conjugate is between 2 and 10 or 2 and 10 or 2 and 5 times higher than the saccharide dose of the Klebsiella pneumoniae O2a O-antigen polysaccharide conjugate. In an embodiment, the saccharide dose of the Klebsiella pneumoniae O3b O-antigen polysaccharide conjugate is between 2 and 10 or 2 and 10 or 2 and 5 times higher than the saccharide dose of the Klebsiella pneumoniae O2agf O-antigen polysaccharide conjugate.

In an embodiment, the saccharide dose of the Klebsiella pneumoniae O3b O-antigen is 5-30 μg or 10-25 μg per human dose. In an embodiment, the saccharide dose of the Klebsiella pneumoniae O3b O-antigen is 16 μg per human dose. In an embodiment, the saccharide dose of the Klebsiella pneumoniae O1v1 O-antigen is 1-10 μg per human dose. In an embodiment, the saccharide dose of the Klebsiella pneumoniae O1v1 O-antigen is 4 μg per human dose. In an embodiment, the saccharide dose of the Klebsiella pneumoniae O2a O-antigen is 1-10 μg per human dose. In an embodiment, the saccharide dose of the Klebsiella pneumoniae O2a O-antigen is 4 μg per human dose. In an embodiment, the saccharide dose of the Klebsiella pneumoniae O2agf O-antigen is 1-10 μg per human dose. In an embodiment, the saccharide dose of the Klebsiella pneumoniae O2agf O-antigen is 4 μg per human dose. In an embodiment, the saccharide dose of the Klebsiella pneumoniae O3b O-antigen is 10-25 μg per human dose and the saccharide dose of each of the Klebsiella pneumoniae O1v1 O-antigen, the Klebsiella pneumoniae O2a O-antigen and the Klebsiella pneumoniae O2agf O-antigen is 1-10 μg per human dose. In an embodiment, the saccharide dose of the Klebsiella pneumoniae O3b O-antigen is 16 μg per human dose and the saccharide dose of each of the Klebsiella pneumoniae O1v1 O-antigen, the Klebsiella pneumoniae O2a O-antigen and the Klebsiella pneumoniae O2agf O-antigen is 4 μg per human dose. In an embodiment, the saccharide dose of the Klebsiella pneumoniae O3b O-antigen is 5-30 μg per human dose and the saccharide dose of each of the Klebsiella pneumoniae O1v1 O-antigen, the Klebsiella pneumoniae O2a O-antigen and the Klebsiella pneumoniae O2agf O-antigen is 1-10 μg per human dose, and the saccharide dose of the Klebsiella pneumoniae O3b O-antigen is less that the saccharid dose of each of the the Klebsiella pneumoniae O1v1 O-antigen, the Klebsiella pneumoniae O2a O-antigen and the Klebsiella pneumoniae O2agf O-antigens. In an embodiment, the volume of the human dose is 0.5 ml.

The present invention provides a multivalent immunogenic composition against subserotypes O1v1, O2a, O2afg and O3b of Klebsiella pneumoniae. In an embodiment, the immunogenic composition comprises O-antigens from subserotypes O1v1, O2a, O2afg and O3b of Klebsiella pneumoniae. Such O-antigens may be in the form of a polysaccharide conjugate where the O-antigen polysaccharide is conjugated (i.e. covalently linked) to a carrier protein. Polysaccharides comprise 2 or more monosaccharides, typically greater than 10 monosaccharides.

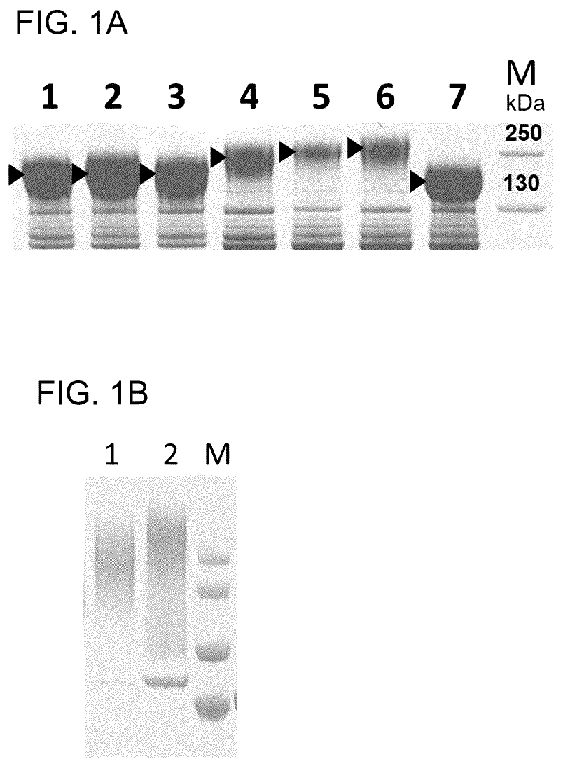

O1-antigens and O2-antigens are built of homopolymers of galactose, i.e. galactans. These O-antigen polysaccharides are part of a family of related structures, which share a D-galactan I backbone (gal-I). D-galactan I has the repeating unit structure: [→3)-β-D-Galf-(1→3)-α-D-Galp-(1-→ (FIG. 1 of Hsieh et al. 2014 Front. Microbiol. 5:608, doi: 10.3389/fmicb.2014.00608) and is the core element of serotype 02a. The O-antigen polysaccharide of serotype O2afg differs from other known O-antigen polysaccharides in Klebsiella spp. in that each of the main-chain Galp residues in the O2afg O-antigen polysaccharide is substituted with an α-(1→4)-linked D-Galp residue, to form a trisaccharide repeating unit, D-galactan III (gal-III) (Kelly et al. (1995) Innate Immun. 2, 131-140). D-galactan III has the repeating unit structure: →3)-β-D-Galf-(1→3)-[α-D-Galp-(1→4)]-α-D-Galp-(1→(Stojkovic et al. 2017 Front. Microbiol. 8:684, doi: 10.3389/fmicb.2017.00684). Kelly et al. J. Biol. Chem. (2019) 294 (28) 10863-10876 further describes the repeat-unit structures of 01 and 02 serogroup antigens. In the case of O1, gal-I is capped by repeats of an antigenically different galactose disaccharide termed D-galactan-II (gal-II). D-galactan II has the repeating unit structure: [→3)-α-D-Galp-(1→3)-β-D-Galp-(1→(FIG. 1 of Hsieh et al. 2014 Front. Microbiol. 5:608, doi: 10.3389/fmicb.2014.00608.) The O-antigen O3b of Klebsiella pneumoniae is described in Guachalla et al. (2017) Scientific Reports 7:6635, 1-13. The O3b O-antigen, has a tri-mannose form, whereas O3 has a penta-mannose form and O3a has a tetra-mannose form. These subtypes have been shown by Guachalla et al. (2017) to be antigenically different.

In an immunogenic composition of the invention the Klebsiella pneumoniae O1v1 O-antigen polysaccharide may have the structure -(D-galactan II)n-(D-galactan I)n-GlcNAc:

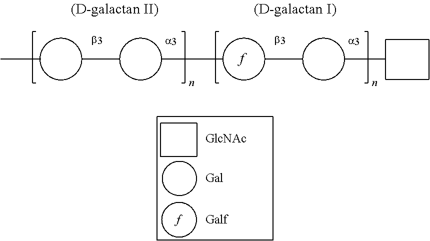

wherein n is the number of repeat units. This structure can also be written as: [→3)-β-D-Galp-(1→3)-α-D-Galp-(1→]n-[→3)-β-D-Galf-(1→3)-α-D-Galp-(1→]n→3)-D-GlcNAc. The number of repeat units for D-galactan II may be different from the number of repeat units for D-galactan I. Optionally the number of repeat units (n) ranges from 4 to 8 or 5 to 7, for example 6 for D-galactan II and the number of repeat units (n) ranges from 2 to 10, 3 to 6, for example 4 for D-galactan I. For example, the number of repeat units (n) may range from 5 to 7 for D-galactan II and the number of repeat units (n) may range from 3 to 5 for D-galactan I. Optionally the ratio of D-galactan II:D-galactan I ranges between 2:1 to 1:50 or 2:1 to 1:2 (e.g. between 1.5:1 to 2:1).

In an immunogenic composition of the invention the Klebsiella pneumoniae O1v1 O-antigen polysaccharide may have the structure -(D-galactan II)n-(D-galactan I)n-GlcNAc:

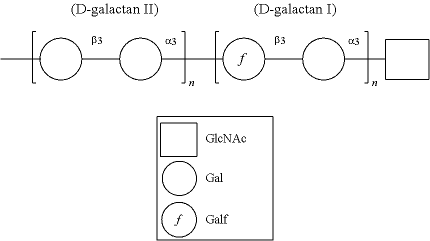

wherein n is the number of repeat units. This structure can also be written as: [→3)-β-D-Galp-(1→3)-α-D-Galp-(1→]n-[→3)-β-D-Galf-(1→3)-α-D-Galp-(1→]n→3)-D-GlcNAc. The number of repeat units for D-galactan II may be different from the number of repeat units for D-galactan I. Optionally the number of repeat units (n) ranges from 8 to 20 or 9 to 14, for example 12 for D-galactan II and the number of repeat units (n) ranges from 2 to 10, 3 to 6, for example 4 for D-galactan I. For example, the number of repeat units (n) may range from 10 to 14 for D-galactan II and the number of repeat units (n) may range from 3 to 5 for D-galactan I. Optionally the ratio of D-galactan II:D-galactan I ranges between 3:1 to 20:1 or 3:1 to 10:1 (e.g. between 3:1 to 5:1).

In an immunogenic composition of the invention the Klebsiella pneumoniae O2a O-antigen polysaccharide may have the structure -(D-galactan I)n-GlcNAc:

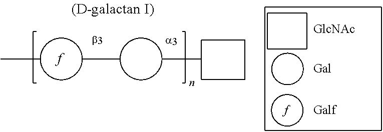

wherein n is the number of repeat units. This structure can also be written as: [→3)-β-D-Galf-(1→3)-α-D-Galp-(1→]n→3)-D-GlcNAc. Optionally the number of repeat units (n) ranges from 10 to 30, e.g. from 15 to 30.

An an immunogenic composition of the invention the Klebsiella pneumoniae O2afg O-antigen polysaccharide may have the structure -(D-galactan III)n-GlcNAc:

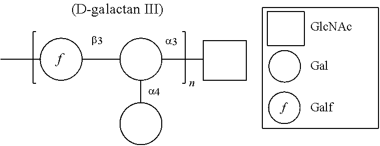

wherein n is the number of repeat units. This structure can also be written as: [→3)-β-D-Galf-(1→3)-[α-D-Galp(1→4)]α-D-Galp(1→]n→3)-D-GlcNAc. Optionally the number of repeat units (n) ranges from 5 to 25 (e.g. from 5 to 15). Optionally the degree of branching ranges from 90-100%.

In an immunogenic composition of the invention the Klebsiella pneumoniae O3b O-antigen polysaccharide may have the structure Me-P-3(Man-α2-Man-α3-Man-α3)n-Man-α3-Man-α3-GlcNAc:

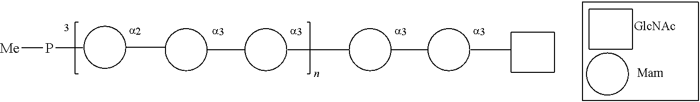

wherein n is the number of repeat units. This structure can also be written as: Me-P-[→3)-α-D-Man(1→2)-α-D-Man(1→3)-α-D-Man(1→]n→3)-α-D-Man (1→3)-α-D-Man (1→3)-D-GlcNAc. Optionally the number of repeat units (n) ranges from 5 to 25 (e.g. from 10 to 20).

An immunogenic composition of the invention may also comprise a pharmaceutically acceptable excipient and/or carrier. Pharmaceutically acceptable excipients and carriers are described, for example, in Remington's Pharmaceutical Sciences, by E. W. Martin, Mack Publishing Co. Easton, PA, 5th Edition (1975). Pharmaceutically acceptable excipients can include a buffer, such as a phosphate buffer (e.g. sodium phosphate). Pharmaceutically acceptable excipients can include a salt, for example sodium chloride. Pharmaceutically acceptable excipients can include a solubilizing/stabilizing agent, for example, polysorbate (e.g. TWEEN 80). Pharmaceutically acceptable excipients can include a preservative, for example 2-phenoxyethanol or thiomersal. Pharmaceutically acceptable excipients can include a carrier such as water or saline.

The present invention provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a carrier protein (e.g. a detoxified Exotoxin A of Pseudomonas aeruginosa (EPA)).

Also provided is a process for making an immunogenic composition of the invention comprising combining a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, and optionally a pharmaceutically acceptable excipient and/or carrier.

### Carrier Proteins

The present invention provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate.

Any carrier protein suitable for use in the production of conjugate vaccines (e.g. bioconjugates for use in vaccines) can be used herein. For example, a nucleotide sequence encoding the carrier protein can be introduced into a host provided herein for the production of a bioconjugate, e.g. a bioconjugate comprising a carrier protein linked to a Klebsiella pneumoniae O-antigen. Exemplary carrier proteins include, without limitation, detoxified Exotoxin A of P. aeruginosa (EPA), CRM197, maltose binding protein (MBP), Diphtheria toxoid, Tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin and detoxified variants thereof, C. jejuni AcrA, Pseudomonas PcrV protein, and C. jejuni natural glycoproteins.

In an embodiments, the carrier protein used in the generation of the bioconjugates described herein are modified, e.g. modified in such a way that the carrier protein is less toxic and/or more susceptible to glycosylation. In a specific embodiment, the carrier proteins used in the generation of the bioconjugates described herein are modified such that the number of glycosylation sites in the carrier proteins is increased in a manner that allows for lower concentrations of the protein to be administered, e.g. in an immunogenic composition, in its bioconjugate form.

The carrier protein may be modified to include 1, 2, 3, 4, 5, 6, 7, 8, 9, 10, or more glycosylation sites than would normally be associated with the carrier protein (e.g. relative to the number of glycosylation sites associated with the carrier protein in its native/natural, e.g. “wild-type” state). In specific embodiments, introduction of glycosylation sites is accomplished by insertion of glycosylation consensus sequences (as described in WO 2006/119987) anywhere in the primary structure of the protein. The carrier protein used herein may comprise a D/E-X-N-Z-S/T (SEQ ID NO: 1) consensus sequence, wherein X and Z are independently any amino acid except proline. Accordingly, the present invention provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a carrier protein comprising an inserted consensus sequence D/E-X-N-Z-S/T (SEQ ID NO: 1) wherein X and Z may be any natural amino acid except proline.

In certain embodiments, the classical 5 amino acid glycosylation consensus sequence (D/E-X-N-Z-S/T (SEQ ID NO: 1)) may be extended by lysine residues for more efficient glycosylation (e.g. K-D/E-X-N-Z-S/T-K (SEQ ID NO: 2)), wherein X and Z are independently any amino acid except proline (preferably wherein X is Q (glutamine), Z is A (alanine). In an embodiment of the invention, one or more amino acids (e.g. 1-7 amino acids, e.g. one amino acid) of the carrier protein amino acid sequence is/are substituted by a five amino acid D/E-X-N-Z-S/T (SEQ ID NO: 1) or by a seven amino acid K-D/E-X-N-Z-S/T-K (SEQ ID NO: 2) (e.g. K-D-Q-N-A-T-K (SEQ ID NO: 3) also referred to as “KDQNATK”) consensus sequence, wherein X and Z are independently any amino acid except proline (preferably wherein X is Q (glutamine), Z is A (alanine)). For example, a single amino acid in the carrier protein amino acid sequence may be substituted (i.e. replaced) with a D/E-X-N-Z-S/T (SEQ ID NO: 1) or K-D/E-X-N-Z-S/T-K (SEQ ID NO: 2) (e.g. K-D-Q-N-A-T-K (SEQ ID NO: 3)) consensus sequence. Alternatively, 2, 3, 4, 5, 6 or 7 amino acids within the carrier protein amino acid sequence may be substituted (i.e. replaced) with a D/E-X-N-Z-S/T (SEQ ID NO: 1) or K-D/E-X-N-Z-S/T-K (SEQ ID NO: 2) consensus sequence, wherein X and Z are independently any amino acid except proline (preferably wherein X is Q (glutamine), Z is A (alanine)) (e.g. K-D-Q-N-A-T-K (SEQ ID NO: 3). The classical 5 amino acid glycosylation consensus sequence (D/E-X-N-Z-S/T (SEQ ID NO: 1)) may also be extended by 1-5 other amino acid residues either side of the consensus sequence for more efficient glycosylation J-D/E-X-N-Z-S/T-U (SEQ ID NO: 4) wherein J and U are independently 1 to 5 naturally occurring amino acid residues (preferably J and U are independently 1 to 5 amino acid residues independently selected from glycine and/or serine, e.g. G-S-G-G-G-D/E-X-N-Z-S/T-G-S-G-G (SEQ ID NO: 5)). Thus, the carrier protein as used herein may comprise consensus sequence(s) selected from: D/E-X-N-Z-S/T (SEQ ID NO: 1), K-D/E-X-N-Z-S/T-K (SEQ ID NO: 2) and/or J-D/E-X-N-Z-S/T-U (SEQ ID NO: 4) wherein X and Z are independently any amino acid except proline (preferably wherein X is Q (glutamine), Z is A (alanine)) and wherein J and U are independently 1 to 5 naturally occurring amino acid residues (preferably J and U are independently 1 to 5 amino acid residues independently selected from glycine and/or serine). For example, the carrier protein as used herein may comprise 3-7 consensus sequence(s) selected from: D/E-X-N-Z-S/T (SEQ ID NO: 1), K-D/E-X-N-Z-S/T-K (SEQ ID NO: 2) and/or J-D/E-X-N-Z-S/T-U (SEQ ID NO: 4) wherein X and Z are independently any amino acid except proline (preferably wherein X is Q (glutamine), Z is A (alanine)) and wherein J and U are independently 1 to 5 naturally occurring amino acid residues (preferably J and U are independently 1 to 5 amino acid residues independently selected from glycine and/or serine).

A combination of consensus sequences selected from: a five amino acid consensus sequence D/E-X-N-Z-S/T (SEQ ID NO: 1), a seven amino acid consensus sequence K-D/E-X-N-Z-S/T-K (SEQ ID NO: 2) and an extended consensus sequence (e.g. J-D/E-X-N-Z-S/T-U (SEQ ID NO: 4)) may be used. For example, a carrier protein may comprise 1, 2, 3, 4 or 5 consensus sequences selected from D/E-X-N-Z-S/T (SEQ ID NO: 1) and K-D/E-X-N-Z-S/T-K (SEQ ID NO: 2), wherein X and Z are independently any amino acid except proline (preferably wherein X is Q (glutamine), Z is A (alanine)), and the carrier protein may further comprise 1 or 2 extended consensus sequences J-D/E-X-N-Z-S/T-U (SEQ ID NO: 4) wherein J and U are independently 1 to 5 naturally occurring amino acid residues (preferably J and U are independently 1 to 5 amino acid residues independently selected from glycine and/or serine, e.g. G-S-G-G-G-D/E-X-N-Z-S/T-G-S-G-G (SEQ ID NO: 5)). Preferably, an extended consensus sequence, such as J-D/E-X-N-Z-S/T-U (SEQ ID NO: 4) or G-S-G-G-G-D/E-X-N-Z-S/T-G-S-G-G (SEQ ID NO: 5) is used where the consensus sequence is added next to the N-terminal or C-terminal amino acid of the EPA protein.

Thus, the present invention also provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a carrier protein comprising 3 to 7 consensus sequence(s) selected from: D/E-X-N-Z-S/T (SEQ ID NO: 1), K-D/E-X-N-Z-S/T-K (SEQ ID NO: 2) and/or J-D/E-X-N-Z-S/T-U (SEQ ID NO: 4), wherein X and Z are independently any amino acid except proline (preferably wherein X is Q (glutamine), Z is A (alanine)) (e.g. K-D-Q-N-A-T-K (SEQ ID NO: 3), and wherein J and U are independently 1 to 5 naturally occurring amino acid residues (preferably J and U are independently 1 to 5 amino acid residues independently selected from glycine and/or serine, e.g. G-S-G-G-G-D/E-X-N-Z-S/T-G-S-G-G (SEQ ID NO: 5)).

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein. In specific embodiments, glycosylation consensus sequences are introduced into specific regions of the carrier protein, e.g. surface structures of the protein, at the N or C termini of the protein, and/or in loops that are stabilized by disulfide bridges at the base of the protein.

In an embodiment, the carrier protein may be a detoxified Exotoxin A of Pseudomonas aeruginosa (EPA). Exotoxin A of Pseudomonas aeruginosa (also known as “EPA”, or “ETA”), is a secreted bacterial toxin, a member of the ADP-ribosyltransferasetoxin family. An EPA protein useful in the invention can be produced by methods known in the art in view of the present disclosure, see for example Ihssen et al. (2010) Microbial Cell Factories 9:61, WO 2006/119987, WO 2009/104074 and WO2015124769A1. Exotoxin A from Pseudomonas aeruginosa strain PA103 was cloned and sequenced by Gray et al. (1984) Proc. Nati. Acad. Sci. USA Vol. 81, pp. 2645-2649. Comparison of the deduced NH₂-terminal amino acid sequence with that determined by sequence analysis of the secreted protein indicated that EPA was made as a 638 amino acid precursor from which a highly hydrophobic leader peptide of 25 amino acids is removed during the secretion process (see FIG. 1 of Gray et al. (1984)). SEQ ID NO: 16 provides the mature EPA amino acid sequence.

```
EPA amino acid sequence
SEQ ID NO: 16
AEEAFDLWNECAKACVLDLKDGVRSSRMSVDPAIADTNGQGVLHYSMVLEGGNDALKLAIDNALSITSDGLTIR
LEGGVEPNKPVRYSYTRQARGSWSLNWLVPIGHEKPSNIKVFIHELNAGNQLSHMSPIYTIEMGDELLAKLARD
ATFFVRAHESNEMQPTLAISHAGVSVVMAQAQPRREKRWSEWASGKVLCLLDPLDGVYNYLAQQRCNLDDTWE
GKIYRVLAGNPAKHDLDIKPTVISHRLHFPEGGSLAALTAHQACHLPLEAFTRHRQPRGWEQLEQCGYPVQRLV
ALYLAARLSWNQVDQVIRNALASPGSGGDLGEAIREQPEQARLALTLAAAESERFVRQGTGNDEAGAASADVVS
LTCPVAAGECAGPADSGDALLERNYPTGAEFLGDGGDVSFSTRGTQNWTVERLLQAHRQLEERGYVFVGYHGT
FLEAAQSIVFGGVRARSQDLDAIWRGFYIAGDPALAYGYAQDQEPDARGRIRNGALLRVYVPRWSLPGFYRTGL
TLAAPEAAGEVERLIGHPLPLRLDAITGPEEEGGRVTILGWPLAERTVVIPSAIPTDPRNVGGDLDPSSIPDKE
QAISALPDYASQPGKPPREDLK
EPA sequence (amino acids 1 to 612 with numbering)
SEQ ID NO: 16
        10 20 30 40 50 60
AEEAFDLWNE CAKACVLDLK DGVRSSRMSV DPAIADTNGQ GVLHYSMVLE GGNDALKLAI
        70 80 90 100 110 120
DNALSITSDG LTIRLEGGVE PNKPVRYSYT RQARGSWSLN WLVPIGHEKP SNIKVFIHEL
      130 140 150 160 170 180
NAGNQLSHMS PIYTIEMGDE LLAKLARDAT FFVRAHESNE MQPTLAISHA GVSVVMAQAQ
      190 200 210 220 230 240
PRREKRWSEW ASGKVLCLLD PLDGVYNYLA QQRCNLDDTW EGKIYRVLAG NPAKHDLDIK
      250 260 270 280 290 300
PTVISHRLHF PEGGSLAALT AHQACHLPLE AFTRHRQPRG WEQLEQCGYP VQRLVALYLA
      310 320 330 340 350 360
ARLSWNQVDQ VIRNALASPG SGGDLGEAIR EQPEQARLAL TLAAAESERF VRQGTGNDEA
      370 380 390 400 410 420
GAASADVVSL TCPVAAGECA GPADSGDALL ERNYPTGAEF LGDGGDVSFS TRGTQNWTVE
      430 440 450 460 470 480
RLLQAHRQLE ERGYVFVGYH GTFLEAAQSI VFGGVRARSQ DLDAIWRGFY IAGDPALAYG
      490 500 510 520 530 540
YAQDQEPDAR GRIRNGALLR VYVPRWSLPG FYRTGLTLAA PEAAGEVERL IGHPLPLRLD
      550 560 570 580 590 600
AITGPEEEGG RVTILGWPLA ERTVVIPSAI PTDPRNVGGD LDPSSIPDKE QAISALPDYA
      610
SQPGKPPRED LK
```

The numbering of the amino acid residues as specified herein, refers to the amino acid position in SEQ ID NO: 16 (or where an amino acid sequence is at least 80%, 85%, 90%, 92%, 95%, 96%, 97%, 98% or 99% identical to SEQ ID NO: 16 to an equivalent position to that of SEQ ID NO: 16 if this sequence was lined up with an amino acid sequence of SEQ ID NO: 16 in order to maximise the sequence identity between the two sequences using Needleman Wunsch algorithm).

Because EPA is a toxin, it needs to be detoxified (i.e. rendered non-toxic to a mammal, e.g. human, when provided at a dosage suitable for protection) before it can be administered in vivo. A detoxified EPA protein may be genetically detoxified (i.e. by mutation). The genetically detoxified sequences may remove undesirable activities such as ADP-ribosyltransferase activity, in order to reduce the toxicity, whilst retaining the ability to induce anti-EPA protective and/or neutralizing antibodies following administration to a human. The genetically detoxified sequences may maintain their immunogenic epitopes. A detoxified EPA protein may be genetically detoxified by one or more point mutations. For example, detoxification can be achieved by mutating and deleting catalytically essential residues, such as substitution of leucine 552 to valine (L552V) and by deletion of glutamic acid-553 (ΔE553), according to Lukac et al. (1988), Infect Immun, 56:3095-3098, and Ho et al. (2006), Hum Vaccin, 2:89-98. Detoxification can be achieved by mutating/deleting the catalytically essential residues L552V ΔE553 using quick change mutagenesis (Stratagene) and phosphorylated oligonucleotides 5′-GAAGGCGGGCGCGTGACCA TTCTCGGC (SEQ ID NO: 40) and 5′-GCCGAGAATGGTCACGCGCCCGCCTTC (SEQ ID NO: 41) resulting in construct pGVXN70. Accordingly, the detoxified EPA protein as used herein may have an amino acid sequence comprising (or consisting) of an amino acid sequence at least 80%, 85%, 90%, 92%, 95%, 96%, 97%, 98% or 99% identical to SEQ ID NO: 16 and having a substitution of leucine 552 to valine (L552V) and deletion of glutamine 553 (ΔE553) with reference to the amino acid sequence of SEQ ID NO: 16 (or an equivalent position in an amino acid sequence at least 80%, 85%, 90%, 92%, 95%, 96%, 97%, 98% or 99% identical to SEQ ID NO: 16).

Detoxification can be measured by determining the inhibition of ADP-ribosyltransferase and cytotoxic activity according to the methodology described in Lukac et al. (1988), Infect Immun, 56:3095-3098, and references cited therein, namely Douglas et al (1987) J. Bacteriol 169:4962-4966 and Douglas et al (1987). A detoxified EPA has ADP-ribosyltransferase and cytotoxic activites lower than wild-type EPA, suitably the same as or less than that of the modified EPA described in Lukac et al (1988) i.e. AE553 EPA (EPA having deletion of glutamic acid-533).

Thus the present invention provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a detoxified Exotoxin A of Pseudomonas aeruginosa (EPA), e.g. a detoxified Exotoxin A of Pseudomonas aeruginosa (EPA) having an amino acid sequence comprising (or consisting) of an amino acid sequence at least 80%, 85%, 90%, 92%, 95%, 96%, 97%, 98% or 99% identical to SEQ ID NO: 16 and having a substitution of leucine 552 to valine (L552V) and deletion of glutamine 553 (ΔE553).

The detoxified Exotoxin A of Pseudomonas aeruginosa (EPA) as used herein may be further modified in that the amino acid sequence comprises one (or more) consensus sequence(s) selected from: D/E-X-N-Z-S/T (SEQ ID NO: 1), K-D/E-X-N-Z-S/T-K (SEQ ID NO: 2) or J-D/E-X-N-Z-S/T-U (SEQ ID NO: 4) wherein X is Q (glutamine), Z is A (alanine), J and U are independently 1 to 5 amino acid residues independently selected from glycine and/or serine, as described above. The one (or more) consensus sequences may each be added next to, or substituted for one or more amino acids selected from specific amino acid residues within the EPA protein (consensus sequence sites). For example, the detoxified Exotoxin A of Pseudomonas aeruginosa (EPA) may comprise 3 to 7 inserted consensus sequences D/E-X-N-Z-S/T, wherein X and Z may be any natural amino acid except proline. Thus the present invention provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a detoxified Exotoxin A of Pseudomonas aeruginosa (EPA) comprises 3 to 7 inserted consensus sequences D/E-X-N-Z-S/T, wherein X and Z may be any natural amino acid except proline. For example, a detoxified Exotoxin A of Pseudomonas aeruginosa (EPA) having an amino acid sequence comprising (or consisting) of an amino acid sequence at least 80%, 85%, 90%, 92%, 95%, 96%, 97%, 98% or 99% identical to SEQ ID NO: 16 and having a substitution of leucine 552 to valine (L552V) and deletion of glutamine 553 (ΔE553) and comprising 3 to 7 inserted consensus sequences D/E-X-N-Z-S/T, wherein X and Z may be any natural amino acid except proline. Thus, the present invention also provides an immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, wherein each of the Klebsiella pneumoniae O1v1, O2a, O2afg and O3b O-antigen polysaccharides are individually conjugated to a detoxified Exotoxin A of Pseudomonas aeruginosa (EPA) carrier protein having an amino acid sequence comprising (or consisting) of an amino acid sequence at least 80%, 85%, 90%, 92%, 95%, 96%, 97%, 98% or 99% identical to SEQ ID NO: 16 modified in having a substitution of leucine 552 to valine (L552V) and deletion of glutamine 553 (ΔE553) and comprising 3 to 7 consensus sequence(s) selected from: D/E-X-N-Z-S/T (SEQ ID NO: 1), K-D/E-X-N-Z-S/T-K (SEQ ID NO: 2) and/or J-D/E-X-N-Z-S/T-U (SEQ ID NO: 4), wherein X and Z are independently any amino acid except proline (preferably wherein X is Q (glutamine), Z is A (alanine)) (e.g. K-D-Q-N-A-T-K (SEQ ID NO: 3), and wherein J and U are independently 1 to 5 naturally occurring amino acid residues (preferably J and U are independently 1 to 5 amino acid residues independently selected from glycine and/or serine, e.g. G-S-G-G-G-D/E-X-N-Z-S/T-G-S-G-G (SEQ ID NO: 5)).

The detoxified Exotoxin A of Pseudomonas aeruginosa (EPA) as used herein may contain four consensus sequences. The detoxified Exotoxin A of Pseudomonas aeruginosa (EPA) as used herein may have an amino acid sequence of SEQ ID NO: 16 or an amino acid sequence at least 80%, 85%, 90%, 92%, 95%, 96%, 97%, 98% or 99% identical to SEQ ID NO: 16 modified in that the amino acid sequence has a substitution of leucine 552 to valine (L552V), a deletion of glutamine 553 (ΔE553) and comprises four consensus sequences, e.g. wherein four consensus sequences are added next to or substituted for four independently selected amino acid residues of SEQ ID NO: 16 or an amino acid sequence at least 80%, 85%, 90%, 92%, 95%, 96%, 97%, 98% or 99% identical to SEQ ID NO: 16. The detoxified Exotoxin A of Pseudomonas aeruginosa (EPA) as used herein may contain four consensus sequences, optionally substituted for amino acid residues Y208, R274, A519 and added next to the N-terminal amino acid of SEQ ID NO: 16 or an amino acid sequence at least 80%, 85%, 90%, 92%, 95%, 96%, 97%, 98% or 99% identical to SEQ ID NO: 16. Preferably, the detoxified Exotoxin A of Pseudomonas aeruginosa (EPA) as used herein may comprise (or consist of) an amino acid sequence which is at least 95%, 96%, 97%, 98%, 99% or 100% identical to SEQ ID NO: 17.

In an embodiment, the carrier protein as used herein further comprises a signal sequence which is capable of directing the carrier protein to the periplasm of a host cell (e.g. bacterium). Signal sequences, including periplasmic signal sequences, are usually removed during translocation of the protein into, for example, the periplasm by signal peptidases (i.e., a mature protein is a protein from which at least the signal sequence has been removed). The signal sequence may be from E. coli flagellin (FlgI) [MIKFLSALILLLVTTAAQA (SEQ ID NO: 6)], E. coli outer membrane porin A (OmpA) [MKKTAIAIAVALAGFATVAQA (SEQ ID NO: 7)], E. coli maltose binding protein (MalE) [MKIKTGARILALSALTTMMFSASALA (SEQ ID NO: 8)], Erwinia carotovorans pectate lyase (PelB) [MKYLLPTAAAGLLLLAAQPAMA (SEQ ID NO: 9)], heat labile E. coli enterotoxin LTIIb [MSFKKIIKAFVIMAALVSVQAHA (SEQ ID NO: 10)], Bacillus subtilis endoxylanase XynA [MFKFKKKFLVGLTAAFMSISMFSATASA (SEQ ID NO: 11)], E. coli DsbA [MKKIWLALAGLVLAFSASA (SEQ ID NO: 12)], TolB [MKQALRVAFGFLILWASVLHA (SEQ ID NO: 13)] or SipA [MKMNKKVLLTSTMAASLLSVASVQAS (SEQ ID NO: 14)]. In a specific embodiment, the signal sequence is from E. coli DsbA [MKKIWLALAGLVLAFSASA (SEQ ID NO: 12)]. Thus, the carrier protein may further comprise a signal sequence which is capable of directing the carrier protein to the periplasm of a host cell (e.g. bacterium), optionally said signal sequence being DsbA (SEQ ID NO: 12). A signal peptide of the protein DsbA from E. coli can be genetically fused to the N-terminus of the mature carrier protein sequence. For example, a plasmid derived from pEC415 [Schulz, H., Hennecke, H., and Thony-Meyer, L., Science, 281, 1197-1200, 1998] containing the DsbA signal peptide code followed by a RNase sequence can be digested (NdeI to EcoRI) to keep the DsbA signal and remove the RNase insert. EPA is then amplified using PCR (forward oligo 5′-AAGCTAGCGCCGCCGAGGAAGCCTTCGACC (SEQ. ID NO. 19) and reverse oligo 5′-AAGAA TTCTCAGTGGTGGTGGTGGTGGTGCTTCAGGTCCTCGCGCGGCGG (SEQ. ID NO. 20)) and digested NheI/EcoRI and ligated to replace the RNase sequence removed previously. The resulting construct (pGVXN69) encodes a protein product with an DsbA signal peptide, the mature carrier sequence and a hexa-histag. For example, a detoxified Exotoxin A of Pseudomonas aeruginosa (EPA) with a DsbA signal sequence having an amino acid sequence comprising (or consisting of) an amino acid sequence which is at least 95%, 96%, 97%, 98%, 99% or 100% identical to SEQ ID NO: 18.

In specific embodiments, the carrier protein expressed by host cells of the invention are expressed from a nucleotide sequence that has been integrated into the genome of the host cell. That is, a nucleotide sequence encoding the carrier protein has been integrated into the host cell genome. Alternatively, the carrier protein expressed in the host cell of the invention is expressed from a plasmid that has been introduced into the host cell.

### Conjugates

The present invention also provides a conjugate (e.g. bioconjugate) comprising a Klebsiella pneumoniae O-antigen polysaccharide selected from O1v1, O2a, O2afg or O3b conjugated to a carrier protein, e.g. wherein the carrier protein is a detoxified Exotoxin A of Pseudomonas aeruginosa (EPA).

In an embodiment, the conjugate (e.g. bioconjugate) comprises (or consists of) a Klebsiella pneumoniae O-antigen polysaccharide selected from O1v1, O2a, O2afg or O3b covalently linked (either directly or through a linker) to a carrier protein, e.g. a detoxified Exotoxin A of Pseudomonas aeruginosa (EPA). In an embodiment, the Klebsiella pneumoniae O-antigen polysaccharide selected from O1v1, O2a, O2afg or O3b is directly linked to the carrier protein, e.g. a detoxified Exotoxin A of Pseudomonas aeruginosa (EPA). In an embodiment, the Klebsiella pneumoniae O-antigen polysaccharide selected from O1v1, O2a, O2afg or O3b is directly linked to an amino acid residue of the carrier protein, e.g. a detoxified Exotoxin A of Pseudomonas aeruginosa (EPA).

In an embodiment, the Klebsiella pneumoniae O-antigen polysaccharide selected from O1v1, O2a, O2afg or O3b is covalently linked to the carrier protein, e.g. a detoxified Exotoxin A of Pseudomonas aeruginosa (EPA) through a chemical linkage obtainable using a chemical conjugation method (i.e. the conjugate is produced by chemical conjugation). The chemical conjugation method may be selected from the group consisting of carbodiimide chemistry, reductive animation, cyanylation chemistry (for example CDAP chemistry), maleimide chemistry, hydrazide chemistry, ester chemistry, and N-hydroysuccinimide chemistry. Conjugates can be prepared by direct reductive amination methods as described in, US200710184072 (Hausdorff) U.S. Pat. No. 4,365,170 (Jennings) and U.S. Pat. No. 4,673,574 (Anderson). Other methods are described in EP-0-161-188, EP-208375 and EP-0-477508. The conjugation method may alternatively rely on activation of the saccharide with 1-cyano-4-dimethylamino pyridinium tetrafluoroborate (CDAP) to form a cyanate ester. Such conjugates are described in PCT published application WO 93/15760 Uniformed Services University and WO 95/08348 and WO 96/29094. See also Chu C. et al. Infect. Immunity, 1983 245 256.

In general the following types of chemical groups on carrier protein, e.g. a detoxified Exotoxin A of Pseudomonas aeruginosa (EPA), can be used for coupling/conjugation:
A) Carboxyl (for instance via aspartic acid or glutamic acid). In one embodiment this group is linked to amino groups on saccharides directly or to an amino group on a linker with carbodiimide chemistry e.g. with EDAC.
B) Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.
C) Sulphydryl (for instance via cysteine). In one embodiment this group is linked to a bromo or chloro acetylated saccharide or linker with maleimide chemistry. In one embodiment this group is activated/modified with bis diazobenzidine.
D) Hydroxyl group (for instance via tyrosine). In one embodiment this group is activated/modified with bis diazobenzidine.
E) Imidazolyl group (for instance via histidine). In one embodiment this group is activated/modified with bis diazobenzidine.
F) Guanidyl group (for instance via arginine).
G) Indolyl group (for instance via tryptophan).

On a saccharide, in general the following groups can be used for a coupling: OH, COOH or NH2. Aldehyde groups can be generated after different treatments such as: periodate, acid hydrolysis, hydrogen peroxide, etc.

Conjugates can be purified by any method known in the art for purification of a protein, for example, by chromatography (e.g. ion exchange, anionic exchange, affinity, and sizing column chromatography), centrifugation, differential solubility, or by any other standard technique for the purification of proteins. See, e.g., Saraswat et al., 2013, Biomed. Res. Int. ID0312709 (p. 1-18); see also the methods described in WO 2009/104074. The actual conditions used to purify a particular conjugate will depend, in past, on the synthesis strategy (e.g., synthetic production vs. recombinant production) and on factors such as net charge, hydrophobicity, and/or hydrophilicity of the bioconjugate.

In an embodiment, the amino acid residue on the carrier protein, e.g. a detoxified Exotoxin A of Pseudomonas aeruginosa (EPA), to which the antigen is linked is selected from the group consisting of: Ala, Arg, Asp, Cys, Gly, Glu, Gln, His, Ile, Leu, Lys, Met, Phe, Pro, Ser, Thr, Trp, Tyr, and Val. Optionally, the amino acid is: an amino acid containing a terminal amine group, a lysine, an arginine, a glutaminic acid, an aspartic acid, a cysteine, a tyrosine, a histidine or a tryptophan. In an embodiment, the amino acid residue on the carrier protein, e.g. a detoxified Exotoxin A of Pseudomonas aeruginosa (EPA), to which the antigen is linked is not an asparagine residue and in this case, the conjugate is typically produced by chemical conjugation. Alternatively, the antigen is linked to an amino acid on the carrier protein, e.g. a detoxified Exotoxin A of Pseudomonas aeruginosa (EPA), selected from asparagine, aspartic acid, glutamic acid, lysine, cysteine, tyrosine, histidine, arginine or tryptophan (e.g. asparagine), and in the case of asparagine the conjugate may be a bioconjugate (for example an enzymatic conjugation using a oligosaccharyltransferase such as PglB). In an embodiment, the amino acid residue on the carrier protein, e.g. a detoxified Exotoxin A of Pseudomonas aeruginosa (EPA), to which the antigen is linked is an asparagine residue. Preferably, the amino acid residue on the modified EPA protein to which the antigen is linked is part of the consensus sequence, e.g. the asparagine in D/E-X-N-Z-S/T (SEQ ID NO: 1), K-D/E-X-N-Z-S/T-K (SEQ ID NO: 2) or J-D/E-X-N-Z-S/T-U (SEQ ID NO: 4) consensus sequence.

The conjugate of the invention may be a conjugate of a a Klebsiella pneumoniae O-antigen polysaccharide selected from O1v1, O2a, O2afg or O3b (e.g. chemical conjugate or bioconjugate). The conjugate of the invention may be a conjugate of an isolated recombinant carrier protein, e.g. a recombinant detoxified Exotoxin A of Pseudomonas aeruginosa (EPA), and a recombinant antigen, e.g. recombinant Klebsiella pneumoniae O-antigen polysaccharide selected from O1v1, O2a, O2afg or O3b (i.e. bioconjugate).

The present invention provides a conjugate (e.g. bioconjugate) wherein the Klebsiella pneumoniae O1v1 O-antigen polysaccharide has the structure -(D-galactan II)n-(D-galactan I)n-GlcNAc:

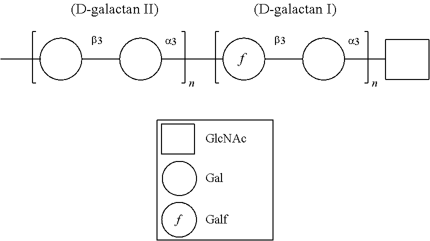

wherein n is the number of repeat units. This structure can also be written as: [→3)-β-D-Galp-(1→3)-α-D-Galp-(1→]n-[→3)-β-D-Galf-(1→3)-α-D-Galp-(1→]n→3)-D-GlcNAc. The number of repeat units for D-galactan II may be different from the number of repeat units for D-galactan I. Optionally the number of repeat units (n) ranges from 4 to 8, 5 to 7, for example 6 for D-galactan II and the number of repeat units (n) ranges from 2 to 10, 3 to 7, for example 4 for D-galactan I. For example, the number of repeat units (n) may range from 5 to 7 for D-galactan II and the number of repeat units (n) may range from 3 to 5 for D-galactan I. Optionally the ratio of D-galactan II:D-galactan I ranges between 2:1 to 1:50 or 2:1 to 1:2 (e.g. between 1.5:1 to 2:1).

A further aspect of the invention is a conjugate (e.g. bioconjugate) wherein the Klebsiella pneumoniae O1v1 O-antigen polysaccharide may have the structure -(D-galactan II)n-(D-galactan I)n-GlcNAc:

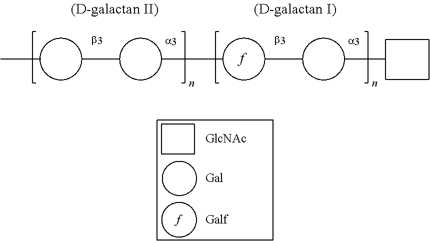

wherein n is the number of repeat units. This structure can also be written as: [→3)-β-D-Galp-(1→3)-α-D-Galp-(1→]n-[→3)-β-D-Galf-(1→3)-α-D-Galp-(1→]n→3)-D-GlcNAc. The number of repeat units for D-galactan II may be different from the number of repeat units for D-galactan I. Optionally the number of repeat units (n) ranges from 8 to 20 or 9 to 14, for example 12 for D-galactan II and the number of repeat units (n) ranges from 2 to 10, 3 to 6, for example 4 for D-galactan I. For example, the number of repeat units (n) may range from 10 to 14 for D-galactan II and the number of repeat units (n) may range from 3 to 5 for D-galactan I. Optionally the ratio of D-galactan II:D-galactan I ranges between 3:1 to 20:1 or 3:1 to 10:1 (e.g. between 3:1 to 5:1).

The present invention provides a conjugate (e.g. bioconjugate) wherein the Klebsiella pneumoniae O2a O-antigen polysaccharide has the structure -(D-galactan I)n-GlcNAc:

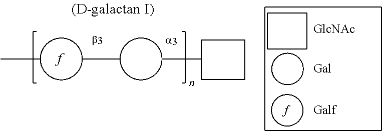

wherein n is the number of repeat units. This structure can also be written as: [→3)-β-D-Galf-(1→3)-α-D-Galp-(1→]n→3)-D-GlcNAc. Optionally the number of repeat units (n) ranges from 10 to 30, e.g. from 15 to 30.

The present invention provides a conjugate (e.g. bioconjugate) wherein the Klebsiella pneumoniae O2afg O-antigen polysaccharide has the structure -(D-galactan III)n-GlcNAc:

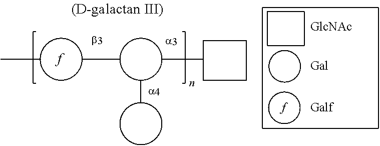

wherein n is the number of repeat units. This structure can also be written as: [→3)-β-D-Galf-(1→3)-[α-D-Galp-(1→4)]-α-D-Galp-(1→]n→3)-D-GlcNAc. Optionally the number of repeat units (n) ranges from 5 to 25 (e.g. from 5 to 15). Optionally the degree of branching ranges from 90-100%.

The present invention provides a conjugate (e.g. bioconjugate) wherein the Klebsiella pneumoniae O3b O-antigen polysaccharide has the structure Me-P-3 (Man-α2-Man-α3-Man-α3)n-Man-α3-Man-α3-GlcNAc:

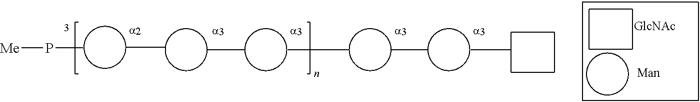

wherein n is the number of repeat units. This structure can also be written as: Me-P-[→3)-α-D-Man(1→2)-α-D-Man (1→3)-α-D-Man(1→]n→3)-α-D-Man(1→3)-α-D-Man(1→3)-D-GlcNAc. Optionally the number of repeat units (n) ranges from 5 to 25 (e.g. from 10 to 20).

The conjugates (e.g. bioconjugate), of the invention are particularly suited for inclusion in immunogenic compositions and vaccines. The present invention also provides an immunogenic composition comprising a conjugate (e.g. bioconjugate) of the invention, and optionally a pharmaceutically acceptable excipient and/or carrier.

### Host Cell

The present invention provides a host cell comprising nucleotide sequences comprising polysaccharide synthesis genes for producing a Klebsiella pneumoniae O-antigen polysaccharide selected from O1v1, O2a, O2afg and O3b and a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline). Thus, the present invention provides a host cell comprising: i) nucleotide sequences comprising polysaccharide synthesis genes for producing a Klebsiella pneumoniae O-antigen polysaccharide selected from O1v1, O2a, O2afg and O3b, optionally integrated into the host cell genome; (ii) a nucleotide sequence encoding a heterologous oligosaccharyl transferase, optionally within a plasmid; (iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and optionally (iv) a nucleotide sequence encoding an ABC transporter, optionally K. pneumoniae genes wzm and wzt, optionally integrated into the host cell genome.

The present invention also provides a host cell comprising:
i) nucleotide sequences comprising polysaccharide synthesis genes for producing a Klebsiella pneumoniae O-antigen polysaccharide selected from O1v1, O2a, O2afg and O3b, optionally integrated into the host cell genome;
ii) a nucleotide sequence encoding a heterologous oligosaccharyl transferase, optionally within a plasmid;
iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and
iv) a nucleotide sequence encoding an ABC transporter, optionally K. pneumoniae genes wzm and wzt, optionally integrated into the host cell genome.

Disclosures of methods for making such host cells which are capable of producing bioconjugates are found in WO 06/119987, WO 09/104074, WO 11/62615, WO 11/138361, WO 14/57109, WO14/72405 and WO16/20499.

Host cells that can be used to produce the bioconjugates of the invention, include archea, prokaryotic host cells, and eukaryotic host cells. In certain embodiments, the host cell is a non-human host cell. Exemplary prokaryotic host cells for use in production of the bioconjugates of the invention include Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. Preferably, the host cell is E. coli (e.g. E. coli K12 W3110).

Where the host cell is E. coli (e.g. E. coli K12 W3110), nucleotide sequences comprising polysaccharide synthesis genes for producing a Klebsiella pneumoniae O-antigen polysaccharide may be integrated into the E. coli O-antigen locus (e.g. the 016-antigen locus of E. coli K12 W3110), optionally in place of one or more genes of the E. coli O-antigen locus. The sequence of the O-antigen cluster of E coli W3110 is reported in GenBank with accession number U03041 (rfb, GenBank U03041). For example, where the host cell is E. coli (e.g. E. coli K12 W3110), the K. pneumoniae genes wbbM, glf, wbbN, and wbbO, may be integrated into E. coli O-antigen locus (e.g. the 016-antigen locus of E. coli K12 W3110), optionally retaining the E. coli O-antigen promoter as a promoter for the polysaccharide synthesis genes. Where the host cell is E. coli (e.g. E. coli K12 W3110), nucleotide sequences comprising polysaccharide synthesis genes for producing a Klebsiella pneumoniae O-antigen polysaccharide may be integrated into the E. coli yeaS locus, optionally in place of the E. coli yeaS gene. The genome of E. coli K12 W3110 is reported in GenBank with accession number NC_007779. The YeaS gene occupies positions 1′881′835 to 1′882′473 (GenBank NC_007779 position 1′881′835 to 1′882′473). For example, where the host cell is E. coli (e.g. E. coli K12 W3110), the K. pneumoniae genes wbbY and wbbZ may be integrated into the E. coli yeaS locus. Thus, the present invention also provides a host cell wherein the host cell is E. coli (e.g. E. coli K12 W3110) and wherein K. pneumoniae genes wbbM, glf, wbbN, and wbbO are integrated into E. coli O-antigen locus (e.g. the 016-antigen locus of E. coli K12 W3110), optionally in place of one or more genes of the E. coli O-antigen locus, and the K. pneumoniae genes wbbY and wbbZ are integrated into the E. coli yeaS locus, optionally in place of the E. coli yeaS gene.

Host cells may be modified to delete or modify genes in the host cell genetic background (genome) that compete or interfere with the synthesis of the polysaccharide of interest (e.g. compete or interfere with one or more heterologous polysaccharide synthesis genes that are recombinantly introduced into the host cell). These genes can be deleted or modified in the host cell background (genome) in a manner that makes them inactive/dysfunctional (i.e. the host cell nucleotide sequences that are deleted/modified do not encode a functional protein or do not encode a protein whatsoever). In an embodiment, when nucleotide sequences are deleted from the genome of the host cells of the invention, they are replaced by a desirable sequence, e.g. a sequence that is useful for polysaccharide synthesis. Exemplary genes that can be deleted in host cells (and, in some cases, replaced with other desired nucleotide sequences) include genes of host cells involved in glycolipid biosynthesis, such as waal (see, e.g. Feldman et al. 2005, PNAS USA 102:3016-3021), the O-antigen cluster (rfb or wb), enterobacterial common antigen cluster (wec), the lipid A core biosynthesis cluster (waa), galactose cluster (gal), arabinose cluster (ara), colonic acid cluster (wc), capsular polysaccharide cluster, undecaprenol-pyrophosphate biosynthesis genes (e.g. uppS (Undecaprenyl pyrophosphate synthase), uppP (Undecaprenyl diphosphatase)), Und-P recycling genes, metabolic enzymes involved in nucleotide activated sugar biosynthesis, enterobacterial common antigen cluster, and prophage O antigen modification clusters like the gtrABS cluster. In an embodiment, one or more of the native waal gene, gtrA gene, gtrB gene, gtrS gene, or a gene or genes from the enterobacterial common antigen cluster (ECA, wec), or a gene, or a gene or genes from the colonic acid cluster (wc) are deleted or functionally inactivated from the genome of a prokaryotic host cell of the invention. In a specific embodiment the host cell of the invention is E. coli, wherein the enterobacterial common antigen cluster (ECA, wec) with the exception of wecA, the colanic acid cluster (wca), and the O-antigen cluster (e.g. the 016-antigen cluster of E. coli K12 W3110) have been deleted. For example, in E. coli K12 W3110 the wec genes are as follows: wecA (UDP-N-acetylglucosamine transferase), wzzE (chain length regulator), wecB (UDP-N-acetylglucosamine epimerase), wecC (UDP-N-acetylmannosamine dehydrogenase), rlmB (TDP-glucose 4,6-dehydratase), rlmA (glucose-1-phosphate thymidylyltransferase), wecD (fucosamine acetyltransferase), wecE (TDP-4-oxo-6-deoxy-D-glucose transaminase), wzxE (ECA translocase), wecF (UDP-N-acetylfucosamine transferase), wzy (ECA polymerase), and wecG (UDP-N-acetylmannosaminuronic acid transferase). In a host cell of the invention, where the native enterobacterial common antigen cluster (ECA, wec) with the exception of wecA is deleted, the genes from wzzE to wecG (i.e. wzzE, wecB, wecC, rlmB, rlmA, wecD, wecE, wzxE, wecF, wzy, and wecG) are deleted. In addition, the native lipopolysaccharide O-antigen ligase waal may be deleted from the host cell of the invention. In addition, the native gtrA gene, gtrB gene and gtrS gene (e.g. the E. coli gtrABS genes) may be deleted from the host cell of the invention.

The host cells of the present invention are engineered to comprise heterologous nucleotide sequences. The host cells of the present invention are engineered to comprise a nucleotide sequence that encodes nucleotide sequences comprising polysaccharide synthesis genes for producing a Klebsiella pneumoniae O-antigen polysaccharide selected from O1v1, O2a, O2afg and O3b.

Polysaccharide synthesis genes encode proteins involved in synthesis of a polysaccharide. The host cells of the invention may comprise one or more nucleotide sequences sufficient for producing a Klebsiella pneumoniae O-antigen polysaccharide selected from O1v1, O2a, O2afg and O3b. Suitably, the present invention provides a host cell comprising nucleotide sequences for producing a Klebsiella pneumoniae O-antigen polysaccharide O1v1, O2a, O2afg or O3b, optionally integrated into the host cell genome. For example the present invention provides a host cell comprising nucleotide sequences for producing a Klebsiella pneumoniae O-antigen polysaccharide O1v1, optionally integrated into the host cell genome. For example the present invention provides a host cell comprising nucleotide sequences for producing a Klebsiella pneumoniae O-antigen polysaccharide 02a, optionally integrated into the host cell genome. For example the present invention provides a host cell comprising nucleotide sequences for producing a Klebsiella pneumoniae O-antigen polysaccharide O2afg, optionally integrated into the host cell genome. For example the present invention provides a host cell comprising nucleotide sequences for producing a Klebsiella pneumoniae O-antigen polysaccharide O3b, optionally integrated into the host cell genome.

Heterologous nucleotide sequences (e.g. nucleotide sequences that encode carrier proteins and/or nucleotide sequences that encode other proteins, e.g. proteins involved in glycosylation) can be introduced into the host cells of the invention using methods such as electroporation, chemical transformation by heat shock, natural transformation, phage transduction, and conjugation. In specific embodiments, heterologous nucleotide sequences are introduced into the host cells of the invention using a plasmid, e.g. the heterologous nucleotide sequences are expressed in the host cells by a plasmid (e.g. an expression vector). In another specific embodiment, heterologous nucleotide sequences are introduced into the host cells of the invention using the method of insertion described in WO14/037585. In an embodiment, the host cell of the present invention comprises one or more nucleotide sequences that comprise polysaccharide synthesis genes which are heterologous to the host cell. In an embodiment, one or more of said nucleotide sequences that comprise polysaccharide synthesis genes which are heterologous to the host cell are integrated into the genome of the host cell. The nucleotide sequences comprising polysaccharide synthesis genes for producing a Klebsiella pneumoniae O-antigen polysaccharide selected from O1v1, O2a, O2afg and O3b may be integrated into the host cell genome. The nucleotide sequences comprising polysaccharide synthesis genes for producing a Klebsiella pneumoniae O1v1, O2a or O2afg O-antigen polysaccharide may comprise K. pneumoniae genes wbbM, gif, wbbN and wbbO. The nucleotide sequences comprising polysaccharide synthesis genes for producing a Klebsiella pneumoniae O-antigen may comprise K. pneumoniae genes wbbM, glf, wbbN and wbbO from a K. pneumoniae strain which expresses an O1v1, O2a or O2afg O-antigen (the wbbM, glf, wbbN and wbbO sequences are identical among several isolates of O1v1, O2a, O2afg). For example, the nucleotide sequences comprising polysaccharide synthesis genes for producing a Klebsiella pneumoniae O-antigen may comprise K. pneumoniae genes wbbM, glf, wbbN and wbbO from a K. pneumoniae strain which expresses an O2a O-antigen. For example, the nucleotide sequences comprising polysaccharide synthesis genes for producing a Klebsiella pneumoniae O-antigen may comprise K. pneumoniae genes wbbM, glf, wbbN and wbbO from a K. pneumoniae strain which expresses an O2afg O-antigen. For example, the nucleotide sequences comprising polysaccharide synthesis genes for producing a Klebsiella pneumoniae O-antigen may comprise K. pneumoniae genes wbbM, glf, wbbN and wbbO from a K. pneumoniae strain which expresses an O1v1 O-antigen. Thus, the present invention provides a host cell wherein the nucleotide sequences comprising polysaccharide synthesis genes for producing a Klebsiella pneumoniae O-antigen polysaccharide comprise K. pneumoniae genes wbbM, glf, wbbN and wbbO. Preferably, the nucleotide sequence for K. pneumoniae gene wbbM comprises (or consists of) a nucleotide sequence at least 95%, 96%, 97%, 98%, 99% or 100% identical to SEQ ID NO: 23. Preferably, the nucleotide sequence for K. pneumoniae gene glf comprises (or consists of) a nucleotide sequence at least 95%, 96%, 97%, 98%, 99% or 100% identical to SEQ ID NO: 24. Preferably, the nucleotide sequence for K. pneumoniae gene wbbN comprises (or consists of) a nucleotide sequence at least 95%, 96%, 97%, 98%, 99% or 100% identical to SEQ ID NO: 25. Preferably, the nucleotide sequence for K. pneumoniae gene wbbO comprises (or consists of) a nucleotide sequence at least 95%, 96%, 97%, 98%, 99% or 100% identical to SEQ ID NO: 26.

In an embodiment, the present invention provides a host cell (e.g. E. coli) comprising:
i) nucleotide sequences for producing a Klebsiella pneumoniae O2a O-antigen polysaccharide comprising K. pneumoniae genes wbbM, glf, wbbN and wbbO, optionally integrated into the host cell genome;
ii) a nucleotide sequence encoding a heterologous oligosaccharyl transferase (e.g. pglB, optionally from Campylobacter jejuni), optionally within a plasmid;
iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and
iv) a nucleotide sequence encoding an ABC transporter, optionally K. pneumoniae genes wzm and wzt, optionally integrated into the host cell genome.

The nucleotide sequences comprising polysaccharide synthesis genes for producing a Klebsiella pneumoniae O2a O-antigen polysaccharide may comprise K. pneumoniae genes wbbM, gif, wbbN and wbbO. Thus, the present invention provides a host cell wherein the nucleotide sequences comprising polysaccharide synthesis genes for producing a Klebsiella pneumoniae O-antigen polysaccharide comprise K. pneumoniae genes wbbM, glf, wbbN and wbbO. The present invention provides a host cell wherein the nucleotide sequences comprising polysaccharide synthesis genes for producing a Klebsiella pneumoniae O2a O-antigen polysaccharide comprise K. pneumoniae genes wbbM, glf, wbbN and wbbO. The nucleotide sequences comprising polysaccharide synthesis genes for producing a Klebsiella pneumoniae O-antigen may comprise K. pneumoniae genes wbbM, glf, wbbN and wbbO from a K. pneumoniae strain which expresses an O2 O-antigen (e.g. from a K. pneumoniae strain which expresses a O2a O-antigen). Preferably wbbM, glf, wbbN and wbbO are from a K. pneumoniae strain which expresses an O2a O-antigen. Preferably, the nucleotide sequence for K. pneumoniae gene wbbM comprises (or consists of) a nucleotide sequence at least 95%, 96%, 97%, 98%, 99% or 100% identical to SEQ ID NO: 23. Preferably, the nucleotide sequence for K. pneumoniae gene glf comprises (or consists of) a nucleotide sequence at least 95%, 96%, 97%, 98%, 99% or 100% identical to SEQ ID NO: 24. Preferably, the nucleotide sequence for K. pneumoniae gene wbbN comprises (or consists of) a nucleotide sequence at least 95%, 96%, 97%, 98%, 99% or 100% identical to SEQ ID NO: 25. Preferably, the nucleotide sequence for K. pneumoniae gene wbbO comprises (or consists of) a nucleotide sequence at least 95%, 96%, 97%, 98%, 99% or 100% identical to SEQ ID NO: 26.

In an embodiment, the present invention provides a host cell (e.g. E. coli) comprising:
i) nucleotide sequences for producing a Klebsiella pneumoniae O2afg O-antigen polysaccharide comprising K. pneumoniae genes wbbM, glf, wbbN, wbbO, gmlA, gmlB and gmlC, optionally integrated into the host cell genome;
ii) a nucleotide sequence encoding a heterologous oligosaccharyl transferase (e.g. pglB, optionally from Campylobacter jejuni), optionally within a plasmid;
iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and
iv) a nucleotide sequence encoding an ABC transporter, optionally K. pneumoniae genes wzm and wzt, optionally integrated into the host cell genome.

The nucleotide sequences comprising polysaccharide synthesis genes for producing a Klebsiella pneumoniae O2afg O-antigen polysaccharide may comprise K. pneumoniae genes wbbM, glf, wbbN, wbbO, gmlA, gmlB and gmlC. Thus, the present invention provides a host cell wherein the nucleotide sequences comprising polysaccharide synthesis genes for producing a Klebsiella pneumoniae O-antigen polysaccharide comprise K. pneumoniae genes wbbM, glf, wbbN, wbbO, gmlA, gmlB and gmlC. The present invention provides a host cell wherein the nucleotide sequences comprising polysaccharide synthesis genes for producing a Klebsiella pneumoniae O2afg O-antigen polysaccharide comprise K. pneumoniae genes wbbM, glf, wbbN, wbbO, gmlA, gmlB and gmlC. The nucleotide sequences comprising polysaccharide synthesis genes for producing a Klebsiella pneumoniae O-antigen may comprise K. pneumoniae genes wbbM, glf, wbbN, wbbO, gmlA, gmlB and gmlC from a K. pneumoniae strain which expresses an 02 O-antigen (e.g. from a K. pneumoniae strain which expresses an O2afg O-antigen). Preferably at least gmlA, gmlB and gmlC are from a K. pneumoniae strain which expresses an O2afg O-antigen. Preferably, the nucleotide sequence encoding K. pneumoniae gene gmlA comprises (or consists of) a nucleotide sequence at least 95%, 96%, 97%, 98%, 99% or 100% identical to SEQ ID NO: 27. Preferably, the nucleotide sequence encoding K. pneumoniae gene gmlB comprises (or consists of) a nucleotide sequence at least 95%, 96%, 97%, 98%, 99% or 100% identical to SEQ ID NO: 28. Preferably, the nucleotide sequence encoding K. pneumoniae gene gmlC comprises (or consists of) a nucleotide sequence at least 95%, 96%, 97%, 98%, 99% or 100% identical to SEQ ID NO: 29.

In an embodiment, the present invention provides a host cell (e.g. E. coli) comprising:
i) nucleotide sequences for producing a Klebsiella pneumoniae O1v1 O-antigen polysaccharide comprising K. pneumoniae genes wbbM, gif, wbbN, wbbO, wbbY and wbbZ, optionally integrated into the host cell genome;
ii) a nucleotide sequence encoding a heterologous oligosaccharyl transferase (e.g. pglB, optionally from Campylobacter jejuni), optionally within a plasmid;
iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and
iv) a nucleotide sequence encoding an ABC transporter, optionally K. pneumoniae genes wzm and wzt, optionally integrated into the host cell genome.

The nucleotide sequences comprising polysaccharide synthesis genes for producing a Klebsiella pneumoniae O1v1 O-antigen polysaccharide may comprise K. pneumoniae genes wbbM, glf, wbbN, wbbO, wbbY and wbbZ. Thus, the present invention provides a host cell wherein the nucleotide sequences comprising polysaccharide synthesis genes for producing a Klebsiella pneumoniae O-antigen polysaccharide comprise K. pneumoniae genes wbbM, glf, wbbN, wbbO, wbbY and wbbZ. The present invention provides a host cell wherein the nucleotide sequences comprising polysaccharide synthesis genes for producing a Klebsiella pneumoniae O1v1 O-antigen polysaccharide comprise K. pneumoniae genes wbbM, glf, wbbN, wbbO, wbbY and wbbZ. The nucleotide sequences comprising polysaccharide synthesis genes for producing a Klebsiella pneumoniae O1v1 O-antigen may comprise K. pneumoniae genes wbbM, glf, wbbN, wbbO, wbbY and wbbZ from a K. pneumoniae strain which expresses an O1 O-antigen (e.g. from a K. pneumoniae strain which expresses an O1v1 O-antigen). Preferably at least wbbY and wbbZ are from a K. pneumoniae strain which expresses an 01v1 O-antigen. Preferably, the nucleotide sequence encoding K. pneumoniae gene wbbY comprises (or consists of) a nucleotide sequence at least 95%, 96%, 97%, 98%, 99% or 100% identical to SEQ ID NO: 30. Preferably, the nucleotide sequence encoding K. pneumoniae gene wbbZ comprises (or consists of) a nucleotide sequence at least 95%, 96%, 97%, 98%, 99% or 100% identical to SEQ ID NO: 31.

In an embodiment, the present invention provides a host cell (e.g. E. coli) comprising:
i) nucleotide sequences for producing a Klebsiella pneumoniae O3b O-antigen polysaccharide comprising K. pneumoniae genes manC, manB, wbdD, wbdA, wbdB and wbdC, optionally integrated into the host cell genome;
ii) a nucleotide sequence encoding a heterologous oligosaccharyl transferase (e.g. pglB, optionally from Campylobacter jejuni), optionally within a plasmid;
iii) a nucleotide sequence that encodes a carrier protein comprising an inserted consensus sequence D/E-X-N-Z-S/T wherein X and Z may be any natural amino acid except proline (e.g. detoxified exotoxin A of Pseudomonas aeruginosa (EPA) comprising an inserted consensus sequence D/E-X-N-Z-S/T wherein X and Z may be any natural amino acid except proline), optionally within a plasmid; and
iv) a nucleotide sequence encoding an ABC transporter, optionally K. pneumoniae genes wzm and wzt, optionally integrated into the host cell genome.

The nucleotide sequences comprising polysaccharide synthesis genes for producing a Klebsiella pneumoniae O3b O-antigen polysaccharide may comprise K. pneumoniae genes manC, manB, wbdD, wbdA, wbdB and wbdC. Thus, the present invention provides a host cell wherein the nucleotide sequences comprising polysaccharide synthesis genes for producing a Klebsiella pneumoniae O-antigen polysaccharide comprise K. pneumoniae genes manC, manB, wbdD, wbdA, wbdB and wbdC. The present invention provides a host cell wherein the nucleotide sequences comprising polysaccharide synthesis genes for producing a Klebsiella pneumoniae O3b O-antigen polysaccharide comprise K. pneumoniae genes manC, manB, wbdD, wbdA, wbdB and wbdC. The nucleotide sequences comprising polysaccharide synthesis genes for producing a Klebsiella pneumoniae O3b O-antigen may comprise K. pneumoniae genes manC, manB, wbdD, wbdA, wbdB and wbdC from a K. pneumoniae strain which expresses an O3 O-antigen (e.g. from a K. pneumoniae strain which expresses an O3b O-antigen). As described in Guachalla et al. (2017) variants in O3 subtypes carry mutations in the mannosyltransferase domains of wbdA. Thus, preferably at least wbdA is from a K. pneumoniae strain which expresses an O3b O-antigen. The nucleotide sequences comprising polysaccharide synthesis genes for producing a Klebsiella pneumoniae O-antigen may comprise K. pneumoniae genes manC, manB, wbdD, wbdA, wbdB and wbdC from a K. pneumoniae strain which expresses an O3b O-antigen. Preferably, the nucleotide sequence encoding K. pneumoniae gene manC comprises (or consists of) a nucleotide sequence at least 95%, 96%, 97%, 98%, 99% or 100% identical to SEQ ID NO: 32. Preferably, the nucleotide sequence encoding K. pneumoniae gene manB comprises (or consists of) a nucleotide sequence at least 95%, 96%, 97%, 98%, 99% or 100% identical to SEQ ID NO: 33. Preferably, the nucleotide sequence encoding K. pneumoniae gene wbdD comprises (or consists of) a nucleotide sequence at least 95%, 96%, 97%, 98%, 99% or 100% identical to SEQ ID NO: 36. Preferably, the nucleotide sequence for K. pneumoniae encoding wbdA comprises (or consists of) a nucleotide sequence at least 95%, 96%, 97%, 98%, 99% or 100% identical to SEQ ID NO: 37. Preferably, the nucleotide sequence encoding K. pneumoniae gene wbdB comprises (or consists of) a nucleotide sequence at least 95%, 96%, 97%, 98%, 99% or 100% identical to SEQ ID NO: 38. Preferably, the nucleotide sequence encoding K. pneumoniae gene wbdC comprises (or consists of) a nucleotide sequence at least 95%, 96%, 97%, 98%, 99% or 100% identical to SEQ ID NO: 39.

The host cells of the present invention are also engineered to comprise a nucleotide sequence that encodes a carrier protein comprising an inserted consensus sequence D/E-X-N-Z-S/T wherein X and Z may be any natural amino acid except proline (e.g. detoxified Exotoxin A of Pseudomonas aeruginosa (EPA) comprising an inserted consensus sequence D/E-X-N-Z-S/T wherein X and Z may be any natural amino acid except proline), optionally within a plasmid. For example, host cells of the present invention may comprise a nucleotide sequence that encodes a detoxified Exotoxin A of Pseudomonas aeruginosa (EPA) having an amino acid sequence comprising (or consisting) of an amino acid sequence at least 80%, 85%, 90%, 92%, 95%, 96%, 97%, 98% or 99% identical to SEQ ID NO: 16 and having a substitution of leucine 552 to valine (L552V) and deletion of glutamine 553 (ΔE553) and comprising 3 to 7 inserted consensus sequences D/E-X-N-Z-S/T, wherein X and Z may be any natural amino acid except proline. For example, host cells of the present invention may comprise a nucleotide sequence that encodes a detoxified Exotoxin A of Pseudomonas aeruginosa (EPA) having an amino acid sequence comprising (or consisting of) an amino acid sequence which is at least 95%, 96%, 97%, 98%, 99% or 100% identical to SEQ ID NO: 17. For example, host cells of the present invention may comprise a nucleotide sequence that encodes a detoxified Exotoxin A of Pseudomonas aeruginosa (EPA) with a signal sequence having an amino acid sequence comprising (or consisting of) an amino acid sequence which is at least 95%, 96%, 97%, 98%, 99% or 100% identical to SEQ ID NO: 18.

Thus, host cells of the invention can produce a bioconjugate comprising a Klebsiella pneumoniae O-antigen polysaccharide selected from O1v1, O2a, O2afg or O3b which is attached to a carrier protein comprising an inserted consensus sequence D/E-X-N-Z-S/T wherein X and Z may be any natural amino acid except proline (e.g. detoxified Exotoxin A of Pseudomonas aeruginosa (EPA) comprising an inserted consensus sequence D/E-X-N-Z-S/T wherein X and Z may be any natural amino acid except proline.

In an embodiment, the host cells may also comprise heterologous nucleotide sequences that are located outside of an O-antigen cluster. For example, nucleotide sequences encoding glycosyltransferases and acetyltransferases that are found outside of O-antigen clusters and that modify recombinant polysaccharides can be introduced into the host cells.

### Oligosaccharyl Transferase

N-linked protein glycosylation (the addition of carbohydrate molecules to an asparagine residue in the polypeptide chain of the target protein) is the most common type of post-translational modification occurring in the endoplasmic reticulum of eukaryotic organisms. The process is accomplished by the enzymatic oligosaccharyltransferase complex (OST) responsible for the transfer of a preassembled oligosaccharide from a lipid carrier (dolichol phosphate) to an asparagine residue of a nascent protein within the conserved sequence Asn-X-Ser/Thr (where X is any amino acid except proline) in the Endoplasmic reticulum.

It has been shown that a bacterium, the food-borne pathogen Campylobacter jejuni, can also N-glycosylate its proteins (Wacker et al. Science. 2002; 298 (5599): 1790-3) due to the fact that it possesses its own glycosylation machinery. The machinery responsible of this reaction is encoded by a cluster called “pgl” (for protein glycosylation). The C. jejuni glycosylation machinery can be transferred to E. coli to allow for the glycosylation of recombinant proteins expressed by the E. coli cells. Previous studies have demonstrated how to generate E. coli strains that can perform N-glycosylation (see, e.g. Wacker et al. Science. 2002; 298 (5599): 1790-3; Nita-Lazar et al. Glycobiology. 2005; 15 (4): 361-7; Feldman et al. Proc Natl Acad Sci USA. 2005; 102 (8): 3016-21; Kowarik et al. EMBO J. 2006; 25 (9): 1957-66; Wacker et al. Proc Natl Acad Sci USA. 2006; 103 (18): 7088-93; International Patent Application Publication Nos. WO2003/074687, WO2006/119987, WO 2009/104074, and WO/2011/06261, and WO2011/138361).

The host cells of the present invention comprise a nucleotide sequence encoding a heterologous oligosaccharyl transferase, optionally within a plasmid. In a specific embodiment, the oligosaccharyl transferase is an oligosaccharyl transferase from Campylobacter. In another specific embodiment, the oligosaccharyl transferase is a pglB, optionally from Campylobacter jejuni (i.e. pglB; see, e.g. Wacker et al. 2002, Science 298:1790-1793; see also, e.g. NCBI Gene ID: 3231775, UniProt Accession No. 086154) SEQ ID NO: 15:

SEQ ID NO: 15
MLKKEYLKNPYLVLFAMIILAYVFSVFCRFYWVWWASEFNEYFFN
NQLMIISNDGYAFAEGARDMIAGFHQPNDLSYYGSSLSALTYWLY
KITPFSFESIILYMSTFLSSLVVIPTILLANEYKRPLMGFVAALL
ASIANSYYNRTMSGYYDTDMLVIVLPMFILFFMVRMILKKDFFSL
IALPLFIGIYLWWYPSSYTLNVALIGLFLIYTLIFHRKEKIFYIA
VILSSLTLSNIAWFYQSAIIVILFALFALEQKRLNFMIIGILGSA
TLIFLILSGGVDPILYQLKFYIFRSDESANLTQGFMYFNVNQTIQ
EVENVDLSEFMRRISGSEIVFLFSLFGFVWLLRKHKSMIMALPIL
VLGFLALKGGLRFTIYSVPVMALGFGFLLSEFKAIMVKKYSQLTS
NVCIVFATILTLAPVFIHIYNYKAPTVFSQNEASLLNQLKNIANR
EDYVVTWWDYGYPVRYYSDVKTLVDGGKHLGKDNFFPSFALSKDE
QAAANMARLSVEYTEKSFYAPQNDILKTDILQAMMKDYNQSNVDL
FLASLSKPDFKIDTPKTRDIYLYMPARMSLIFSTVASFSFINLDT
GVLDKPFTFSTAYPLDVKNGEIYLSNGVVLSDDFRSFKIGDNVVS
VNSIVEINSIKQGEYKITPIDDKAQFYIFYLKDSAIPYAQFILMD
KTMFNSAYVQMFFLGNYDKNLFDLVINSRDAKVFKLKI

Thus host cells of the present invention may comprise a nucleotide sequence encoding pglB, optionally pglB from Campylobacter jejuni, optionally a nucleotide sequence encoding pglB from Campylobacter jejuni having a sequence at least 95%, 96%, 97%, 98%, 99% or 100% identical to SEQ ID NO: 15, optionally within a plasmid.

### Chain Elongation

In host cells of the present invention chain elongation is carried out by multifunctional glycosyltransferases (i.e. the nucleotide sequences comprising polysaccharide synthesis genes for producing a Klebsiella pneumoniae O-antigen polysaccharide as described herein). Accordingly, there is no need for a polymerase and it is not necessary to introduce a heterologous polymerase. Thus host cells of the present invention may lack a nucleotide sequence encoding a heterologous polymerase (e.g. wzy).

### ABC Transporters

The host cells of the present invention may be engineered to comprise a nucleotide sequence that encodes an ABC transporter. The ABC transporter transfers the repeating units of a polysaccharide from the cytoplasm into the periplam of host cells (e.g. E. coli). For example, host cells of the present invention may comprise a nucleotide sequence encoding K. pneumoniae genes wzm and wzt. The nucleotide sequences encoding an ABC transporter may comprise K. pneumoniae genes wzm and wzt from a K. pneumoniae strain which expresses O2 O-antigen (e.g. from a K. pneumoniae strain which expresses an O2a O-antigen), e.g. for synthesis of a Klebsiella pneumoniae O2a O-antigen. The nucleotide sequences encoding an ABC transporter may comprise K. pneumoniae genes wzm and wzt from a K. pneumoniae strain which expresses O2 O-antigen (e.g. from a K. pneumoniae strain which expresses an O2afg O-antigen), e.g. for synthesis of a Klebsiella pneumoniae O2afg O-antigen. The nucleotide sequences encoding an ABC transporter may comprise K. pneumoniae genes wzm and wzt from a K. pneumoniae strain which expresses O1 O-antigen (e.g. from a K. pneumoniae strain which expresses an O1v1 O-antigen), e.g. for synthesis of a Klebsiella pneumoniae O1v1 O-antigen. For example, the amino acid sequence encoding K. pneumoniae gene wzm comprises (or consists of) a nucleotide sequence at least 95%, 96%, 97%, 98%, 99% or 100% identical to SEQ ID NO: 21. For example, the amino acid sequence encoding K. pneumoniae gene wzt comprises (or consists of) a nucleotide sequence at least 95%, 96%, 97%, 98%, 99% or 100% identical to SEQ ID NO: 22. The nucleotide sequences encoding an ABC transporter may comprise K. pneumoniae genes wzm and wzt from a K. pneumoniae strain which expresses O3 O-antigen (e.g. from a K. pneumoniae strain which expresses an O3b O-antigen), e.g. for synthesis of a Klebsiella pneumoniae O3b O-antigen. For example, the nucleotide sequence encoding K. pneumoniae gene wzm comprises (or consists of) a nucleotide sequence at least 95%, 96%, 97%, 98%, 99% or 100% identical to SEQ ID NO: 34. For example, the nucleotide sequence encoding K. pneumoniae gene wzt comprises (or consists of) a nucleotide sequence at least 95%, 96%, 97%, 98%, 99% or 100% identical to SEQ ID NO: 35. The nucleotide sequence that encodes an ABC transporter may be introduced as part of the Klebsiella pneumoniae O-antigen cluster for a particular serotype.

The nucleotide sequence encoding the ABC transporter may be integrated into the host cell genome. The nucleotide sequence encoding the ABC transporter may co-localised with the nucleotide sequences comprising polysaccharide synthesis genes for producing a Klebsiella pneumoniae O-antigen polysaccharide O1v1, O2a, O2afg or O3b within the host cell genome. Thus, the present invention provides a host cell wherein nucleotide sequences comprising polysaccharide synthesis genes for producing a Klebsiella pneumoniae O-antigen polysaccharide O1v1, O2a, O2afg or O3b and the nucleotide sequence encoding an ABC transporter are integrated into the host cell genome, optionally co-localized.

### Accessory Enzymes

In an embodiment, nucleotide sequences encoding one or more accessory enzymes are introduced into the host cells of the invention. Thus, a host cell of the invention may further comprise one or more of these accessory enzymes. Such nucleotide sequences encoding one or more accessory enzymes can be either plasmid-borne or integrated into the genome of the host cells of the invention. Exemplary accessory enzymes include, without limitation, epimerases (see e.g. WO2011/062615), branching, modifying (e.g. to add cholins, glycerolphosphates, pyruvates), amidating, acetylating, formylating enzymes.

### Bioconjugates

The present invention provides a bioconjugate comprising a Klebsiella pneumoniae O-antigen polysaccharide, in particular a Klebsiella pneumoniae O-antigen polysaccharide selected from O1v1, O2a, O2afg or O3b, conjugated to a carrier protein, wherein the carrier protein is a detoxified Exotoxin A of Pseudomonas aeruginosa (EPA).

The present invention provides a bioconjugate comprising a Klebsiella pneumoniae O-antigen polysaccharide O1v1 optionally having the structure -(D-galactan II)n-(D-galactan I)n-GlcNAc:

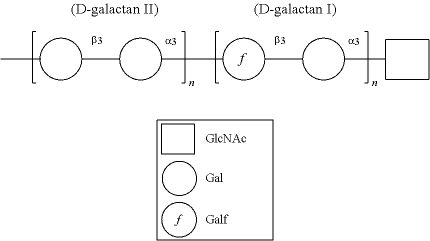

wherein n is the number of repeat units. This structure can also be written as: [→3)-β-D-Galp-(1→3)-α-D-Galp-(1→]n-[→3)-β-D-Galf-(1→3)-α-D-Galp-(1→]n→3)-D-GlcNAc. The number of repeat units for D-galactan II may be different from the number of repeat units for D-galactan I. Optionally the number of repeat units (n) ranges from 4 to 8 or 5 to 7, for example 6 for D-galactan II and the number of repeat units (n) ranges from 2 to 10 or 3 to 7, for example 4 for D-galactan I. For example, the number of repeat units (n) may range from 5 to 7 for D-galactan II and the number of repeat units (n) may range from 3 to 5 for D-galactan I. Optionally the ratio of D-galactan II:D-galactan I ranges between 2:1 to 1:50 or 2:1 to 1:2 (e.g. between 1.5:1 to 2:1).

The present invention provides a bioconjugate comprising a Klebsiella pneumoniae O1v1 O-antigen polysaccharide optionally having the structure -(D-galactan II)n-(D-galactan I)n-GlcNAc:

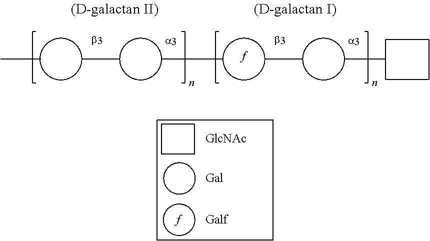

wherein n is the number of repeat units. This structure can also be written as: [→3)-β-D-Galp-(1→3)-α-D-Galp-(1→]n-[→3)-β-D-Galf-(1→3)-α-D-Galp-(1→]n→3)-D-GlcNAc. The number of repeat units for D-galactan II may be different from the number of repeat units for D-galactan I. Optionally the number of repeat units (n) ranges from 8 to 20 or 9 to 14, for example 12 for D-galactan II and the number of repeat units (n) ranges from 2 to 10, 3 to 6, for example 4 for D-galactan I. For example, the number of repeat units (n) may range from 10 to 14 for D-galactan II and the number of repeat units (n) may range from 3 to 5 for D-galactan I. Optionally the ratio of D-galactan II:D-galactan I ranges between 3:1 to 20:1 or 3:1 to 10:1 (e.g. between 3:1 to 5:1).

The present invention provides a bioconjugate comprising a Klebsiella pneumoniae O-antigen polysaccharide O2a has the structure -(D-galactan I)n-GlcNAc:

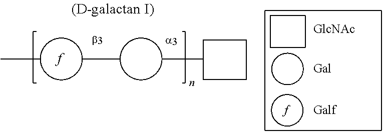

wherein n is the number of repeat units. This structure can also be written as: [→3)-β-D-Galf-(1→3)-α-D-Galp-(1→]n→3)-D-GlcNAc. Optionally the number of repeat units (n) ranges from 10 to 30, e.g. from 15 to 30.

The present invention provides a bioconjugate comprising a Klebsiella pneumoniae O-antigen polysaccharide O2afg has the structure -(D-galactan III)n-GlcNAc:

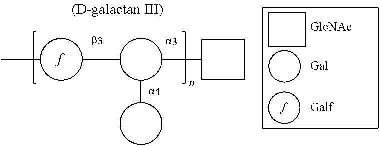

wherein n is the number of repeat units. This structure can also be written as: [→3)-β-D-Galf-(1→3)-[α-D-Galp-(1→4)]-α-D-Galp-(1→]n→3)-D-GlcNAc. Optionally the number of repeat units (n) ranges from 5 to 25 (e.g. from 5 to 15). Optionally the degree of branching ranges from 90-100%.

The present invention provides a bioconjugate comprising a Klebsiella pneumoniae O-antigen polysaccharide O3b has the structure Me-P-3(Man-α2-Man-α3-Man-α3)n-Man-α3-Man-α3-GlcNAc:

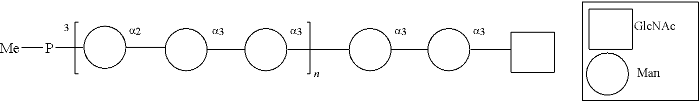

wherein n is the number of repeat units. This structure can also be written as: Me-P-[→3)-α-D-Man(1→2)-α-D-Man(1→3)-α-D-Man (1→]n→3)-α-D-Man(1→3)-α-D-Man(1→3)-D-GlcNAc. Optionally the number of repeat units (n) ranges from 5 to 25 (e.g. from 10 to 20).

The present invention provides a bioconjugate according to the invention wherein the detoxified Exotoxin A of Pseudomonas aeruginosa (EPA) comprises 3 to 7 inserted consensus sequences D/E-X-N-Z-S/T (SEQ ID NO. 1), wherein X and Z may be any natural amino acid except proline. For example, a detoxified Exotoxin A of Pseudomonas aeruginosa (EPA) having an amino acid sequence comprising (or consisting) of an amino acid sequence at least 80%, 85%, 90%, 92%, 95%, 96%, 97%, 98% or 99% identical to SEQ ID NO: 16 and having a substitution of leucine 552 to valine (L552V) and deletion of glutamine 553 (ΔE553) and comprising 3 to 7 inserted consensus sequences D/E-X-N-Z-S/T, wherein X and Z may be any natural amino acid except proline. For example, a detoxified Exotoxin A of Pseudomonas aeruginosa (EPA) having an amino acid sequence comprising (or consisting of) an amino acid sequence which is at least 95%, 96%, 97%, 98%, 99% or 100% identical to SEQ ID NO: 17. Thus, the present invention provides a bioconjugate wherein the detoxified Exotoxin A of Pseudomonas aeruginosa (EPA) comprises 3 to 7 inserted consensus sequences D/E-X-N-Z-S/T, wherein X and Z may be any natural amino acid except proline, optionally comprising (or consisting of) an amino acid sequence which is at least 95%, 96%, 97%, 98%, 99% or 100% identical to SEQ ID NO: 17.

The Klebsiella pneumoniae O-antigen may be linked to an amino acid on the modified EPA protein selected from asparagine, aspartic acid, glutamic acid, lysine, cysteine, tyrosine, histidine, arginine or tryptophan (e.g. asparagine). Bioconjugates, as described herein, have advantageous properties over chemical conjugates of antigen-carrier protein, in that they require less chemicals in manufacture and are more consistent in terms of the final product generated.

A further aspect of the invention is a process for producing a bioconjugate that comprises (or consists of) a Klebsiella pneumoniae O-antigen polysaccharide selected from O1v1, O2a, O2afg or O3b, conjugated to a carrier protein, wherein the carrier protein is a detoxified Exotoxin A of Pseudomonas aeruginosa (EPA), said process comprising (i) culturing the host cell of the invention under conditions suitable for the production of glycoproteins and (ii) isolating the bioconjugate produced by said host cell, optionally isolating the bioconjugate from a periplasmic extract from the host cell. There is thus provided a process for producing a bioconjugate comprising (i) culturing the host cell of the invention under conditions suitable for the production of glycoproteins and (ii) isolating the bioconjugate. There is also provided a process for producing a bioconjugate comprising (i) culturing the host cell of the invention under conditions suitable for the production of glycoproteins and (ii) isolating the bioconjugate from a periplasmic extract from the host cell.

For example, bioconjugates can be made using the shakeflask process, e.g. in a LB shake flask. In aspect of the invention, a fed-batch process for the production of recombinant glycosylated proteins in bacteria can be used to produce bioconjugates of the invention. The aim is to increase glycosylation efficiency and recombinant protein yield per cell and while maintaining simplicity and reproducibility in the process. Bioconjugates of the present invention can be manufactured on a commercial scale by developing an optimized manufacturing method using typical E. coli production processes. Various types of feed strategies, such as batch, chemostat and fed-batch can be used.

The bioconjugates of the invention can be purified for example, by chromatography (e.g. ion exchange, anionic exchange, affinity, and sizing column chromatography), centrifugation, differential solubility, or by any other standard technique for the purification of proteins. See, e.g. Saraswat et al. 2013, Biomed. Res. Int. ID #312709 (p. 1-18); see also the methods described in WO 2009/104074. Further, the bioconjugates may be fused to heterologous polypeptide sequences described herein or otherwise known in the art to facilitate purification.

The present invention also provides an immunogenic composition comprising the conjugate (e.g. bioconjugate) of the invention, and optionally a pharmaceutically acceptable excipient and/or carrier. The invention provides an immunogenic composition comprising a Klebsiella pneumoniae O1v1 O-antigen polysaccharide conjugate (e.g. bioconjugate) of the invention. The invention provides an immunogenic composition comprising a Klebsiella pneumoniae O2a O-antigen polysaccharide conjugate (e.g. bioconjugate) of the invention. The invention provides an immunogenic composition comprising a Klebsiella pneumoniae O2afg O-antigen polysaccharide conjugate (e.g. bioconjugate) of the invention. The invention provides an immunogenic composition comprising a Klebsiella pneumoniae O3b O-antigen polysaccharide conjugate (e.g. bioconjugate) of the invention.

### Analytical Methods

Various methods can be used to analyze the structural compositions and sugar chain lengths of the bioconjugates of the invention and to determine glycosylation site usage.

Hydrazinolysis can be used to analyze glycans. First, polysaccharides are released from their protein carriers by incubation with hydrazine according to the manufacturer's instructions (Ludger Liberate Hydrazinolysis Glycan Release Kit, Oxfordshire, UK). The nucleophile hydrazine attacks the glycosidic bond between the polysaccharide and the carrier protein and allows release of the attached glycans. N-acetyl groups are lost during this treatment and have to be reconstituted by re-N-acetylation. The free glycans are purified on carbon columns and subsequently labeled at the reducing end with the fluorophor 2-amino benzamide. See Bigge J C, Patel T P, Bruce J A, Goulding P N, Charles S M, Parekh R B: Nonselective and efficient fluorescent labeling of glycans using 2-amino benzamide and anthranilic acid. Anal Biochem 1995, 230 (2): 229-238. The labeled polysaccharides are separated on a GlycoSep-N column (GL Sciences) according to the HPLC protocol of Royle et al., See Royle L, Mattu T S, Hart E, Langridge J I, Merry A H, Murphy N, Harvey D J, Dwek R A, Rudd P M: An analytical and structural database provides a strategy for sequencing O-glycans from microgram quantities of glycoproteins. Anal Biochem 2002, 304 (1): 70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Alternatively, high mass MS and size exclusion HPLC can be applied to measure the size of the complete bioconjugates.

Yield may be measured as carbohydrate amount derived from a liter of bacterial production culture grown in a bioreactor under controlled and optimized conditions. After purification of bioconjugate, the carbohydrate yields can be directly measured by either the anthrone assay or ELISA using carbohydrate specific antisera. Indirect measurements are possible by using the protein amount (measured by BCA, Lowry, or bardford assays) and the glycan length and structure to calculate a theoretical carbohydrate amount per gram of protein. In addition, yield can also be measured by drying the glycoprotein preparation from a volatile buffer and using a balance to measure the weight.

Various methods can be used to analyze the conjugates of the invention including, for example, SDS-PAGE or capillary gel electrophoresis. Polymer length is defined by the number of repeat units that are linearly assembled. This means that the typical ladder like pattern is a consequence of different repeat unit numbers that compose the glycan. Thus, two bands next to each other in SDS PAGE (or other techniques that separate by size) differ by only a single repeat unit. These discrete differences are exploited when analyzing glycoproteins for glycan size: the unglycosylated carrier protein and the bioconjugate with different polymer chain lengths separate according to their electrophoretic mobilities. The first detectable repeat unit number (n₁) and the average repeat unit number (n_{average}) present on a bioconjugate are measured. These parameters can be used to demonstrate batch to batch consistency or polysaccharide stability, for example.

Glycosylation site usage may be quantified by, for example, glycopeptide LC-MS/MS: conjugates are digested with protease(s), and the peptides are separated by a suitable chromatographic method (C18, Hydrophilic interaction HPLC HILIC, GlycoSepN columns, SE HPLC, AE HPLC), and the different peptides are identified using MS/MS. This method can be used with or without previous sugar chain shortening by chemical (smith degradation) or enzymatic methods. Quantification of glycopeptide peaks using UV detection at 215 to 280 nm allows relative determination of glycosylation site usage. In another embodiment, site usage may be quantified by size exclusion HPLC: Higher glycosylation site usage is reflected by an earlier elution time from a SE HPLC column. In yet another embodiment, site usage may be quantified by quantitative densitometry of purified bioconjugates stained with Coomassie Briliant Blue following sodium dodecyl sulfate-polyacrylamide gel electrophoresis (SDS-PAGE).

### Vaccines

### Vaccines

The present invention also provides an immunogenic composition (e.g., a vaccine composition) optionally comprising an adjuvant.

The term “adjuvant” refers to a compound that when administered in conjunction with or as part of an immunogenic composition of vaccine of the invention augments, enhances and/or boosts the immune response to a conjugate (e.g. bioconjugate) of the invention, but when the compound is administered alone does not generate an immune response to the conjugate (e.g. bioconjugate). Adjuvants can enhance an immune response by several mechanisms including, e.g. lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS01 (GlaxoSmithKline), and saponins, such as QS21 (see Kensil et al. in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al. N. Engl. J. Med. 336, 86-91 (1997)). In an embodiment, the adjuvant is an oil in water emulsion comprises a metabolisable oil, a tocol and an emulsifier. In an embodiment, the metabolizable oil is squalene. In an embodiment the tocol is alpha-tocoferol. In an embodiment, the emulsifier is polyoxyethylene sorbitan monooleate. In an embodiment, the oil in water emulsion comprises 1-10, 2-10, 3-9, 4-8. 5-7, or 5-6 mg metabolisable oil, per dose. In an embodiment, the oil in water emulsion comprises 0.5-11, 1-11, 2-10, 3-9, 4-8, 5-7, 5-6 mg tocol per dose. In an embodiment, the oil in water emulsion comprises 0.1-5, 0.2-5, 0.3-4, 0.4-3 or 2-3 mg emulsifying agent per dose.

Also provided is a method of making the immunogenic composition of the invention comprising the step of mixing the conjugate (e.g. bioconjugate) of the invention with a pharmaceutically acceptable excipient and/or carrier and an adjuvant. Vaccine preparation is generally described in Vaccine Design (“The subunit and adjuvant approach” (eds Powell M. F. & Newman M. J.) (1995) Plenum Press New York).

The immunogenic compositions of the invention can be included in a container, pack, or dispenser together with instructions for administration.

The immunogenic compositions or vaccines of the invention can be stored before use, e.g. the compositions can be stored frozen (e.g. at about −20° C. or at about −70° C.); stored in refrigerated conditions (e.g. at about 4° C.); or stored at room temperature. The immunogenic compositions or vaccines of the invention may be stored in solution or lyophilized. In an embodiment, the solution is lyophilized in the presence of a sugar such as sucrose, trehalose or lactose. In another embodiment, the vaccines of the invention are lyophilized and extemporaneously reconstituted prior to use.

### Administration and Dosage

Immunogenic compositions or vaccines of the invention may be used to protect or treat a subject (e.g. mammal), by means of administering said immunogenic composition or vaccine via systemic or mucosal route. These administrations may include injection via the intramuscular (IM), intraperitoneal, intradermal (ID) or subcutaneous (SC) routes; or via mucosal administration to the oral/alimentary, respiratory, genitourinary tracts.

In one aspect, the immunogenic composition or vaccine of the invention is administered by the intramuscular delivery route. Intramuscular administration may be to the thigh or the upper arm. Injection is typically via a needle (e.g. a hypodermic needle), but needle-free injection may alternatively be used. A typical intramuscular dose is 0.5 ml.

In another aspect, the immunogenic composition or vaccine of the invention is administered by the intradermal administration. Human skin comprises an outer “horny” cuticle, called the stratum corneum, which overlays the epidermis. Underneath this epidermis is a layer called the dermis, which in turn overlays the subcutaneous tissue. The conventional technique of intradermal injection, the “mantoux procedure”, comprises steps of cleaning the skin, and then stretching with one hand, and with the bevel of a narrow gauge needle (26 to 31 gauge) facing upwards the needle is inserted at an angle of between 10 to 15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

In another aspect, the immunogenic composition or vaccine of the invention is administered by the intranasal administration. Typically, the immunogenic composition or vaccine is administered locally to the nasopharyngeal area, e.g. without being inhaled into the lungs. It is desirable to use an intranasal delivery device which delivers the immunogenic composition or vaccine formulation to the nasopharyngeal area, without or substantially without it entering the lungs. Suitable devices for intranasal administration of the vaccines according to the invention are spray devices. Suitable commercially available nasal spray devices include ACCUSPRAY™ (Becton Dickinson).

Immunogenic compositions comprise an immunologically effective amount of one or more Klebsiella pneumoniae polysaccharide conjugates (e.g. bioconjugates) of the invention, as well as any other components. By “immunologically effective amount”, it is meant that the administration of that amount to an individual, either as a single dose or as part of a series is effective for treatment or prevention of a Klebsiella pneumoniae infection, disease or condition. This amount varies depending on the health and physical condition of the individual to be treated, age, the degree of protection desired, the formulation of the vaccine and other relevant factors.

The amount of conjugate (e.g. bioconjugate) in each immunogenic composition or vaccine dose is selected as an amount which induces an immunoprotective response without significant, adverse side effects in typical vaccines. Such amount will vary depending upon which specific immunogen is employed and how it is presented. The content of conjugate (e.g. bioconjugate) will typically be in the range 1-100 μg, suitably 5-50 μg.

### Prophylactic and Therapeutic Uses

The present invention also provides an immunogenic composition of the invention, or the vaccine of the invention, for use in medicine.

Provided herein are methods (and uses) of inducing an immune response in a subject against Klebsiella pneumoniae, comprising administering to the subject a conjugate (e.g. bioconjugate) of the invention an immunogenic composition of the invention or a vaccine of the invention. The immunogenic composition of the invention or the vaccine of the invention comprises conjugate(s) (e.g. bioconjugate(s)) of Klebsiella pneumoniae O1v1 O-antigen polysaccharide, Klebsiella pneumoniae O1v2 O-antigen polysaccharide, Klebsiella pneumoniae O2a O-antigen polysaccharide conjugate, Klebsiella pneumoniae O2afg O-antigen polysaccharide and/or a Klebsiella pneumoniae O3b O-antigen polysaccharide, wherein each of the Klebsiella pneumoniae O1v1, O1v2, O2a, O2afg and O3b O-antigen polysaccharides are individually conjugated to a carrier protein. In an embodiment, the conjugate(s) is/are bioconjugate(s). In one embodiment, said subject has bacterial infection at the time of administration. In another embodiment, said subject does not have a bacterial infection at the time of administration.

Thus, the present invention provides a method of inducing an immune response to Klebsiella pneumoniae in a subject, the method comprising administering a therapeutically or prophylactically effective amount of the immunogenic composition of the invention, or the vaccine of the invention, to a subject (e.g. human) in need thereof. The present invention also provides an immunogenic composition of the invention, or the vaccine of the invention, for use in inducing an immune response to Klebsiella pneumoniae in a subject (e.g. human). The present invention also provides an immunogenic composition of the invention for use in the manufacture of a medicament for inducing an immune response to Klebsiella pneumoniae in a subject (e.g. human). Also provided herein are methods (and uses) of inducing the production of opsonophagocytic antibodies in a subject (e.g. human) against Klebsiella pneumoniae, comprising administering to the subject a conjugate (e.g. bioconjugate) of the invention an immunogenic composition of the invention or a vaccine of the invention. In an embodiment, the conjugate (e.g. bioconjugate) of the invention an immunogenic composition of the invention or a vaccine of the invention can be used to induce the production of opsonophagocytic antibodies in a subject (e.g. human) against Klebsiella pneumoniae.

The present invention also provides methods of treating and/or preventing a Klebsiella pneumoniae infection in a subject comprising administering to the subject a conjugate (e.g. bioconjugate) of the invention. The conjugate (e.g. bioconjugate) may be in the form of an immunogenic composition or vaccine. Thus, the present invention provides a method of treating or preventing a Klebsiella pneumoniae infection, disease or condition in a subject, the method comprising administering a therapeutically or prophylactically effective amount of the immunogenic composition of the invention, or the vaccine of the invention, to a subject (e.g. human) in need thereof. The present invention also provides an immunogenic composition of the invention, or the vaccine of the invention, for use in treating or preventing a Klebsiella pneumoniae infection, disease or condition in a subject (e.g. human). The present invention also provides an immunogenic composition of the invention for use in the manufacture of a medicament for treating or preventing a Klebsiella pneumoniae infection, disease or condition in a subject (e.g. human).

### Cross-Reactivity

The present inventors have found that sera obtained by immunization with certain Klebsiella O-antigen serotypes are cross-reactive and can thus provide cross-protection against other Klebsiella O-antigen serotypes despite the antigenic differences between the serotypes. The present inventors have found that antisera generated by immunization with a conjugate of Klebsiella pneumoniae O1v1 O-antigen polysaccharide bind the corresponding subserotype Klebsiella pneumoniae O1v2 O-antigen polysaccharide and that antisera generated by immunization with a conjugate of Klebsiella pneumoniae O1v2 O-antigen polysaccharide bind the corresponding subserotype Klebsiella pneumoniae O1v1 O-antigen polysaccharide. The cross protection between these two distinct subserotypes allows a vaccine comprising either an O1v1 or O1v2 serotype to protect against the other serotype. This means that the multivalent immunogenic composition or vaccine of the invention can offer a broader protection against the range of Klebsiella pneumoniae serotypes, covering greater than 60% of non-resistant strains and greater than 75% of resistant strains (with cross-reactivity it is estimated to cover 80.4% of non-resistant strains and 81.9% of resistant strains). The advantages of such an immunogenic composition/vaccine include minimizing the cost of goods and minimizing the likelihood of interference of one antigen over another.

Thus the present invention provides a method of treating or preventing a Klebsiella pneumoniae infection, disease or condition associated with an O1v2 strain of Klebsiella pneumoniae in a subject, the method comprising administering a therapeutically or prophylactically effective amount of an immunogenic composition of the invention or the vaccine of the invention, comprising a conjugate (e.g. bioconjugate) of a non-pyruvylated or less than 50%, 40%, 30%, 20%, 10%, 5% or 1% pyruvylated Klebsiella pneumoniae O1v1 O-antigen polysaccharide, to a subject (e.g. human) in need thereof. The present invention also provides an immunogenic composition of the invention or a vaccine of the invention, comprising a conjugate (e.g. bioconjugate) of a non-pyruvylated or less than 50%, 40%, 30%, 20%, 10%, 5% or 1% pyruvylated Klebsiella pneumoniae O1v1 O-antigen polysaccharide, for use in treating or preventing a Klebsiella pneumoniae infection, disease or condition associated with an O1v2 strain of Klebsiella pneumoniae in a subject (e.g. human). The present invention also provides an immunogenic composition of the invention comprising a conjugate (e.g. bioconjugate) of a non-pyruvylated or less than 50%, 40%, 30%, 20%, 10%, 5% or 1% pyruvylated Klebsiella pneumoniae O1v1 O-antigen polysaccharide, for use in the manufacture of a medicament for treating or preventing a Klebsiella pneumoniae infection, disease or condition associated with an O1v2 strain of Klebsiella pneumoniae in a subject (e.g. human).

In an embodiment, the immunogenic composition of the invention, or vaccine of the invention comprising a conjugate (e.g. bioconjugate of non-pyruvylated or less than 50%, 40%, 30%, 20%, 10%, 5% or 1% pyruvylated Klebsiella pneumoniae O1v1 O-antigen polysaccharide), when administered to a subject (e.g. human), is able to induce the formation of antibodies capable of binding to Klebsiella pneumoniae O1v2 as measured by ELISA assay. In the ELISA (Enzyme-linked Immunosorbent Assay) method, antibodies from the sera of vaccinated subjects are incubated with polysaccharides which have been adsorbed to a solid support. The bound antibodies are detected using enzyme-conjugated secondary detection antibodies.

In an embodiment, the immunogenic composition of the invention, or the vaccine of the invention, does not comprise Klebsiella pneumoniae O1v2 O-antigen polysaccharide. Thus the present invention provides a method of treating or preventing a Klebsiella pneumoniae infection, disease or condition associated with an O1v2 strain of Klebsiella pneumoniae in a subject, the method comprising administering a therapeutically or prophylactically effective amount of an immunogenic composition of the invention or a vaccine of the invention, comprising a conjugate (e.g. bioconjugate) of a non-pyruvylated or less than 50%, 40%, 30%, 20%, 10%, 5% or 1% pyruvylated Klebsiella pneumoniae O1v1 O-antigen polysaccharide and which does not comprise Klebsiella pneumoniae O1v2 O-antigen polysaccharide, to a subject (e.g. human) in need thereof. The present invention also provides an immunogenic composition of the invention or a vaccine of the invention, comprising a conjugate (e.g. bioconjugate) of a non-pyruvylated or less than 50%, 40%, 30%, 20%, 10%, 5% or 1% pyruvylated Klebsiella pneumoniae O1v1 O-antigen polysaccharide and which does not comprise Klebsiella pneumoniae O1v2 O-antigen polysaccharide, for use in treating or preventing a Klebsiella pneumoniae infection, disease or condition associated with an O1v2 strain of Klebsiella pneumoniae in a subject (e.g. human). The present invention also provides an immunogenic composition of the invention comprising a conjugate (e.g. bioconjugate) of a non-pyruvylated or less than 50%, 40%, 30%, 20%, 10%, 5% or 1% pyruvylated Klebsiella pneumoniae O1v1 O-antigen polysaccharide or Klebsiella pneumoniae O1v1 O-antigen which is less than 50%, 40%, 30%, 20%, 10%, 5% or 1% pyruvylated and which does not comprise Klebsiella pneumoniae O1v2 O-antigen polysaccharide, for use in the manufacture of a medicament for treating or preventing a Klebsiella pneumoniae infection, disease or condition associated with an O1v2 strain of Klebsiella pneumoniae in a subject (e.g. human).

Embodiments of the invention are further described in the subsequent numbered paragraphs:
1. An immunogenic composition comprising a Klebsiella pneumoniae O1v1 O-antigen polysaccharide conjugate (for example a bioconjugate), a Klebsiella pneumoniae O2a O-antigen polysaccharide conjugate (for example a bioconjugate), a Klebsiella pneumoniae O2afg O-antigen polysaccharide conjugate (for example a bioconjugate) and a Klebsiella pneumoniae O3b O-antigen polysaccharide conjugate (for example a bioconjugate), wherein each of the Klebsiella pneumoniae O1v1, O2a, O2afg and O3b O-antigen polysaccharides are individually conjugated to a carrier protein (e.g. a detoxified Exotoxin A of Pseudomonas aeruginosa (EPA)) wherein the Klebsiella pneumoniae O3b O-antigen polysaccharide conjugate is present in said immunogenic composition at a higher saccharide dose than at least one of the Klebsiella pneumoniae O1v1 O-antigen polysaccharide conjugate, Klebsiella pneumoniae O2a O-antigen polysaccharide conjugate or Klebsiella pneumoniae O2afg O-antigen polysaccharide conjugate.
2. The immunogenic composition according to paragraph 1 wherein the Klebsiella pneumoniae O3b O-antigen polysaccharide conjugate is present at a higher saccharide dose than the Klebsiella pneumoniae O1v1 O-antigen polysaccharide conjugate.
3. The immunogenic composition according to paragraph 1 or 2 wherein the Klebsiella pneumoniae O3b O-antigen polysaccharide conjugate is present at a higher saccharide dose than the Klebsiella pneumoniae O2a O-antigen polysaccharide conjugate.
4. The immunogenic composition according to paragraph 1 or 2 or 3 wherein the Klebsiella pneumoniae O3b O-antigen polysaccharide conjugate is present at a higher saccharide dose than the Klebsiella pneumoniae O2afg O-antigen polysaccharide conjugate.
5. The immunogenic composition according to paragraphs 1~4 wherein the Klebsiella pneumoniae O3b O-antigen polysaccharide conjugate is present at a higher saccharide dose than each of the Klebsiella pneumoniae O1v1 O-antigen polysaccharide conjugate, Klebsiella pneumoniae O2a O-antigen polysaccharide conjugate and Klebsiella pneumoniae O2afg O-antigen polysaccharide conjugate.
6. The immunogenic composition according to any one of paragraphs 1-5 wherein the saccharide dose of the Klebsiella pneumoniae O3b O-antigen polysaccharide conjugate is at least 1.5, 2, 3, 4, 5, 6, 7, 8, 9 or 10 times higher than the saccharide dose of the Klebsiella pneumoniae O1v1 O-antigen polysaccharide conjugate.
7. The immunogenic composition according to any one of paragraphs 1-6 wherein the saccharide dose of the Klebsiella pneumoniae O3b O-antigen polysaccharide conjugate is at least 1.5, 2, 3, 4, 5, 6, 7, 8, 9 or 10 times higher than the saccharide dose of the Klebsiella pneumoniae O2a O-antigen polysaccharide conjugate.
8. The immunogenic composition according to any one of paragraphs 1-7 wherein the saccharide dose of the Klebsiella pneumoniae O3b O-antigen polysaccharide conjugate is at least 1.5, 2, 3, 4, 5, 6, 7, 8, 9 or 10 times higher than the saccharide dose of the Klebsiella pneumoniae O2afg O-antigen polysaccharide conjugate.
9. The immunogenic composition according to any one of paragraphs 1-8 wherein the saccharide dose of the Klebsiella pneumoniae O3b O-antigen polysaccharide conjugate is at least 1.5, 2, 3, 4, 5, 6, 7, 8, 9 or 10 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate.
10. The immunogenic composition according to any one of paragraphs 1-9 wherein the saccharide dose of the Klebsiella pneumoniae O3b O-antigen polysaccharide conjugate is between 1.5 and 20 times higher than the saccharide dose of the Klebsiella pneumoniae O1v1 O-antigen polysaccharide conjugate.
11. The immunogenic composition according to any one of paragraphs 1-10 wherein the saccharide dose of the Klebsiella pneumoniae O3b O-antigen polysaccharide conjugate is between 1.5 and 20 times higher than the saccharide dose of the Klebsiella pneumoniae O2a O-antigen polysaccharide conjugate.
12. The immunogenic composition according to any one of paragraphs 1-11 wherein the saccharide dose of the Klebsiella pneumoniae O3b O-antigen polysaccharide conjugate is between 1.5 and 20 times higher than the saccharide dose of the Klebsiella pneumoniae O2agf O-antigen polysaccharide conjugate.
13. The immunogenic composition according to any one of paragraphs 1-12 wherein the saccharide dose of the Klebsiella pneumoniae O3b O-antigen polysaccharide conjugate is between 1.5 and 20 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate.
14. The immunogenic composition according to any one of paragraphs 1-13 wherein the saccharide dose of the Klebsiella pneumoniae O3b O-antigen polysaccharide conjugate is between 2 and 10 times higher than the saccharide dose of the Klebsiella pneumoniae O1v1 O-antigen polysaccharide conjugate.
15. The immunogenic composition according to any one of paragraphs 1-14 wherein the saccharide dose of the Klebsiella pneumoniae O3b O-antigen polysaccharide conjugate is between 2 and 10 times higher than the saccharide dose of the Klebsiella pneumoniae O2a O-antigen polysaccharide conjugate.
16. The immunogenic composition according to any one of paragraphs 1-15 wherein the saccharide dose of the Klebsiella pneumoniae O3b O-antigen polysaccharide conjugate is between 2 and 10 times higher than the saccharide dose of the Klebsiella pneumoniae O2agf O-antigen polysaccharide conjugate.
17. The immunogenic composition according to any one of paragraphs 1-16 wherein the saccharide dose of the Klebsiella pneumoniae O3b O-antigen polysaccharide conjugate is between 2 and 10 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate.
18. The immunogenic composition according to any one of paragraphs 1-17 wherein the saccharide dose of the Klebsiella pneumoniae O3b O-antigen polysaccharide conjugate is between 2 and 5 times higher than the saccharide dose of the Klebsiella pneumoniae O1v1 O-antigen polysaccharide conjugate.
19. The immunogenic composition according to any one of paragraphs 1-18 wherein the saccharide dose of the Klebsiella pneumoniae O3b O-antigen polysaccharide conjugate is between 2 and 5 times higher than the saccharide dose of the Klebsiella pneumoniae O2a O-antigen polysaccharide conjugate.
20. The immunogenic composition according to any one of paragraphs 1-19 wherein the saccharide dose of the Klebsiella pneumoniae O3b O-antigen polysaccharide conjugate is between 2 and 5 times higher than the saccharide dose of the Klebsiella pneumoniae O2agf O-antigen polysaccharide conjugate.
21. The immunogenic composition according to any one of paragraphs 1-20 wherein the saccharide dose of the Klebsiella pneumoniae O3b O-antigen polysaccharide conjugate is between 2 and 5 times higher than each of the saccharide doses of the Klebsiella pneumoniae O1v1 O-antigen polysaccharide conjugate, the Klebsiella pneumoniae O2a O-antigen polysaccharide conjugate and the Klebsiella pneumoniae O2afg O-antigen polysaccharide conjugate.
22. The immunogenic composition according to any one of paragraphs 1-21 wherein the saccharide dose of the Klebsiella pneumoniae O3b O-antigen is 10-25 mg per human dose.
23. The immunogenic composition according to any one of paragraphs 1-22 wherein the saccharide dose of the Klebsiella pneumoniae O1v1 O-antigen is 1-10 mg per human dose.
24. The immunogenic composition according to any one of paragraph 1-23 wherein the saccharide dose of the Klebsiella pneumoniae O2a O-antigen is 1-10 mg per human dose.
25. The immunogenic composition according to any one of paragraphs 1-24 wherein the saccharide dose of the Klebsiella pneumoniae O2agf O-antigen is 1-10 mg per human dose.
26. The immunogenic composition according to any one of paragraphs 1-25 wherein the saccharide dose of the Klebsiella pneumoniae O3b O-antigen is 10-25 mg per human dose and wherein the saccharide dose of each of the Klebsiella pneumoniae O1v1 O-antigen, the Klebsiella pneumoniae O2a O-antigen and the Klebsiella pneumoniae O2agf O-antigen is 1-10 mg per human dose.
27. The immunogenic composition according to any one of paragraphs 1-26 wherein the conjugate is a bioconjugate and/or the volume of the human dose is 0.5 ml.
28. The immunogenic composition according to any one of paragraphs 1-27 wherein the carrier protein comprises an inserted consensus sequence D/E-X-N-Z-S/T wherein X and Z may be any natural amino acid except proline.
29. The immunogenic composition according to any one of paragraphs 1-28 wherein the carrier protein is a detoxified Exotoxin A of Pseudomonas aeruginosa (EPA).
30. The immunogenic composition according to paragraph 29 wherein the detoxified Exotoxin A of Pseudomonas aeruginosa (EPA) comprises 3 to 7 inserted consensus sequences D/E-X-N-Z-S/T, wherein X and Z may be any natural amino acid except proline, optionally comprising (or consisting of) an amino acid sequence which is at least 95%, 96%, 97%, 98%, 99% or 100% identical to SEQ ID NO: 17.
31. The immunogenic composition according to any of paragraphs 1-30 wherein the Klebsiella pneumoniae O1v1 O-antigen polysaccharide has the structure: -(D-galactan II)n-(D-galactan I)n-GlcNAc

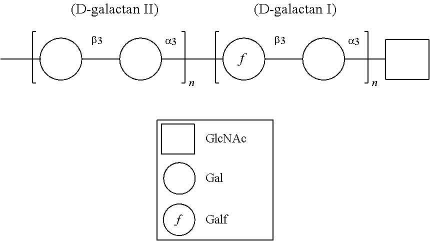

optionally wherein the number of repeat units n ranges from 5 to 7 for D-galactan II and the number of repeat units n ranges from 2 to 10 for D-galactan I and optionally wherein the ratio of D-galactan II:D-galactan I ranges between 2:1 to 1:50.
32. The immunogenic composition according to any one of paragraphs 1-31 wherein the Klebsiella pneumoniae O1v1 O-antigen polysaccharide is less than 50%, 40%, 30%, 20% or 10% pyruvylated or not capped with a pyruvate group.
33. The immunogenic composition according to paragraph 32 wherein the Klebsiella pneumoniae O1v1 O-antigen polysaccharide has the structure: -(D-galactan II)n-(D-galactan I)n-GlcNAc:

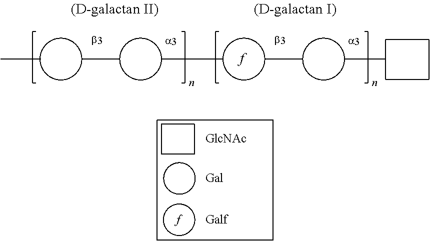

optionally wherein the number of repeat units n ranges from 8 to 20 (optionally 10-14) for D-galactan II and the number of repeat units n ranges from 2 to 10 for D-galactan I and optionally wherein the ratio of D-galactan II:D-galactan I ranges between 3:1 to 10:1.
34. The immunogenic composition according to any of paragraphs 1-33 wherein the Klebsiella pneumoniae O2a O-antigen polysaccharide has the structure -(D-galactan I)n-GlcNAc:

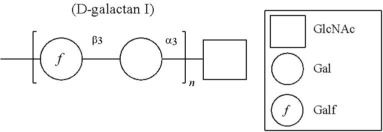

optionally wherein the number of repeat units n ranges from 10 to 30.
35. The immunogenic composition according to any of paragraphs 1-34 wherein the Klebsiella pneumoniae O2afg O-antigen polysaccharide has the structure -(D-galactan III)n-GlcNAc:

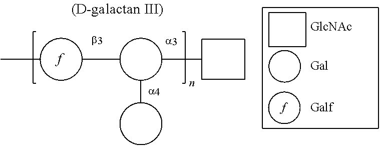

optionally wherein the number of repeat units n ranges from 5 to 25 and optionally wherein the degree of branching ranges from 90-100%.
36. The immunogenic composition according to any of paragraphs 1-35 wherein the Klebsiella pneumoniae O3b O-antigen polysaccharide has the structure Me-P-3 (Man-α2-Man-α3-Man-α3)n-Man-α3-Man-α3-GlcNAc:

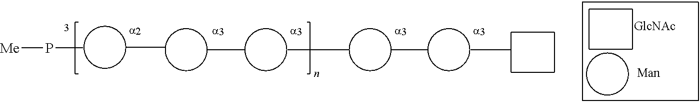

optionally wherein the number of repeat units n ranges from 5 to 25.
37. The immunogenic composition according to any one of paragraphs 1-36 comprising a pharmaceutically acceptable excipient and/or carrier.
38. A process for making an immunogenic composition of any of paragraphs 1-37, comprising combining a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate such that the Klebsiella pneumoniae O3b O-antigen polysaccharide conjugate is present in said immunogenic composition at a higher saccharide dose than at least one of the Klebsiella pneumoniae O1v1 O-antigen polysaccharide conjugate, Klebsiella pneumoniae O2a O-antigen polysaccharide conjugate or Klebsiella pneumoniae O2afg O-antigen polysaccharide conjugate, and optionally adding a pharmaceutically acceptable excipient and/or carrier.
39. A vaccine comprising the immunogenic composition of any of paragraphs 1-37 and optionally an adjuvant.
40. The vaccine according to paragraph 39 comprising an adjuvant which is an oil-in-water emulsion comprising a metabolizable oil, a tocol and an emulsifier.
41. The vaccine according to paragraph 40 wherein the metabolizable oil is squalene.
42. The vaccine according to paragraph 40 or 41 wherein the tocol is alpha-tocoferol.
43. The vaccine according to any one of paragraphs 40-42 wherein the emulsifier is polyoxyethylene sorbitan monooleate.
44. The vaccine according to any one of paragraphs 40-43 wherein the oil in water emulsion comprises 1-10, 2-10, 3-9, 4-8. 5-7, or 5-6 mg metabolisable oil, per dose.
45. The vaccine according to any one of paragraphs 40-44 wherein the oil in water emulsion comprises 0.5-11, 1-11, 2-10, 3-9, 4-8, 5-7, 5-6 mg tocol per dose.
46. The vaccine according to any one of paragraphs 40-45 wherein the oil in water emulsion comprises 0.1-5, 0.2-5, 0.3-4, 0.4-3 or 2-3 mg emulsifying agent per dose.
47. A method of inducing an immune response to Klebsiella pneumoniae in a subject, the method comprising administering a therapeutically or prophylactically effective amount of the immunogenic composition of paragraphs 1-37, or the vaccine of any one of paragraphs 39-46, to a subject (e.g. human) in need thereof.
48. A method of treating or preventing a Klebsiella pneumoniae infection, disease or condition in a subject, the method comprising administering a therapeutically or prophylactically effective amount of the immunogenic composition of any one of paragraphs 1-37, or the vaccine of any one of paragraphs 39-46, to a subject (e.g. human) in need thereof.
49. A method of treating or preventing a Klebsiella pneumoniae infection, disease or condition associated with an O1v2 strain of Klebsiella pneumoniae in a subject, the method comprising administering a therapeutically or prophylactically effective amount of the immunogenic composition of any one of paragraphs 1-37 or the vaccine of any one of paragraphs 39-46, comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, to a subject (e.g. human) in need thereof.
50. A method of inducing an immune response to Klebsiella pneumoniae in a subject, the method comprising administering a therapeutically or prophylactically effective amount of an immunogenic composition comprising a Klebsiella pneumoniae O1v1 O-antigen polysaccharide conjugate (for example a bioconjugate), a Klebsiella pneumoniae O2a O-antigen polysaccharide conjugate (for example a bioconjugate), a Klebsiella pneumoniae O2afg O-antigen polysaccharide conjugate (for example a bioconjugate) and a Klebsiella pneumoniae O3b O-antigen polysaccharide conjugate (for example a bioconjugate), wherein each of the Klebsiella pneumoniae O1v1, O2a, O2afg and O3b O-antigen polysaccharides are individually conjugated to a carrier protein to a subject (e.g. human) in need thereof followed by a subsequent step of administering an immunogenic composition comprising a Klebsiella pneumoniae O3b O-antigen polysaccharide conjugate which does not contain a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate to the subject.
51. The immunogenic composition of any one of paragraphs 1-37, or the vaccine of any one of paragraphs 39-46, for use in inducing an immune response to Klebsiella pneumoniae in a subject (e.g. human).
52. The immunogenic composition of any one of paragraphs 1-37, or the vaccine of any one of paragraphs 39-46, for use in treating or preventing a Klebsiella pneumoniae infection, disease or condition in a subject (e.g. human).
53. The immunogenic composition of any one of paragraphs 1-37 or the vaccine of any one of paragraphs 39-46, comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, for use in treating or preventing a Klebsiella pneumoniae infection, disease or condition associated with an O1v2 strain of Klebsiella pneumoniae in a subject (e.g. human).
54. An immunogenic composition for use as a booster in inducing an immune response to Klebsiella pneumoniae in a subject (e.g. human) comprising a Klebsiella pneumoniae O3b O-antigen polysaccharide conjugate (for example a bioconjugate) but not comprising a Klebsiella pneumoniae O1v1 O-antigen polysaccharide conjugate (for example a bioconjugate), a Klebsiella pneumoniae O2a O-antigen polysaccharide conjugate (for example a bioconjugate) or a Klebsiella pneumoniae O2afg O-antigen polysaccharide conjugate (for example a bioconjugate).
55. The immunogenic composition of any one of paragraphs 1-37 for use in the manufacture of a medicament for inducing an immune response to Klebsiella pneumoniae in a subject (e.g. human).
56. The immunogenic composition of any one of paragraphs 1-37 for use in the manufacture of a medicament for treating or preventing a Klebsiella pneumoniae infection, disease or condition in a subject (e.g. human).
57. The immunogenic composition of any one of paragraphs 1-37 comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate and a Klebsiella pneumoniae O3b O-antigen polysaccharide conjugate, for use in the manufacture of a medicament for treating or preventing a Klebsiella pneumoniae infection, disease or condition associated with an O1v2 strain of Klebsiella pneumoniae in a subject (e.g. human).

In order that this invention may be better understood, the following examples are set forth. These examples are for purposes of illustration only, and are not to be construed as limiting the scope of the invention in any manner.

### EXAMPLES

### Example 1: Generation of Klebsiella pneumoniae Pyruvylated O1v1 (wbbZ+), Non-Pyruvylated O1v1 (wbbZ−), O2a, O2Afg, O3b O-Antigen-EPA Bioconjugates

### Bioconjugate-Producing Strains' Construction

In order to optimally produce glycan-protein bioconjugates, E. coli K12 W3110 benefits from the following genetic modifications: i. deletion of genomic cluster involved in glycan biosynthesis and transport which could potentially negatively affect the expression of recombinant glycans; ii. introduction of the target glycan's biosynthetic genes; iii. introduction of the protein carrier's encoding gene; iv. introduction of the olygosaccharyl transferase PglB encoding gene. The construction of glycan-production strains for the four K. pneumoniae serotypes varies therefore only with respect of the genes required for the glycan biosynthesis.

An E. coli K12 W3110-derivative strain devoid of potential interfering pathways was constructed by subsequent replacements of the targeted gene clusters with an FRT sites-flanked selection marker via A-Red homologous recombination followed by FLP recombinase-catalysed marker removal (Kuhlman and Cox Nucleic Acids Res. 2010 April; 38 (6): e92; or WO 19/30234). Five homologous recombination/marker removal steps were carried out, removing genomic sequences of:
i. O16 O-antigen cluster (rfb or wb, GenBank NCBI Reference Sequence NC_007779.1 (dated Jun. 7, 2020) position 2′114′113 to 2′103′814),
ii. colanic acid cluster (wca, GenBank NCBI Reference Sequence NC_007779.1 (dated Jun. 7, 2020) position 2′138′241 to 2′118′033),
iii. ECA cluster retaining wecA (wec, GenBank NCBI Reference Sequence NC_007779.1 (dated Jun. 7, 2020) position 3′666′604 to 3′656′725),
iv. O16wzz2 or cld (GenBank NCBI Reference Sequence NC_007779.1 (dated Jun. 7, 2020) position 2′099′458 to 2′100′438), and
v. gtrABS or yfdGHI (GenBank NCBI Reference Sequence NC_007779.1 (dated Jun. 7, 2020) position 2′473′301 to 2′475′908).

This strain is here referred as “clean strain”.

This “clean strain” was the target for the insertion of the clusters. Genes wzm, wzt, wbbM, glf, wbbN, wbbO from K. pneumoniae (GenBank Accession No. CP052562.1 (dated May 4, 2020) position 1′695′622 to 1′702′243) were inserted into the 016 O-antigen cluster together with a selection marker (which was later removed) using known techniques (T E Kuhlman and E C Cox. Nucleic Acids Res. 2010 April; 38 (6): e92.), originating the O2a glycan-producing strain. The transcription of the inserted genes was driven by the native E. coli O-antigen cluster promoter and was therefore constitutive.

Genes gmlABCas in K. pneumoniae (GenBank Accession No. CP052562.1 (dated May 4, 2020) position 1′706′431 to 1′703′615) were inserted into the ECA cluster (retaining wecA) of the 02a glycan-producing strain together with a selection marker (which was later removed) using known techniques (T E Kuhlman and E C Cox. Nucleic Acids Res. 2010 April; 38 (6): e92.), originating the O2afg glycan-producing strain. The transcription of the inserted genes was driven by the native E. coli ECA cluster promoter and was therefore constitutive.

Genes wbbY and wbbZ and the DNA region in between them featuring a transcription promoter as in K. pneumoniae (GenBank Accession No. LT174607.1 (dated May 9, 2017) position 5′605 to 8′734) were used to replace the gene yeaS (GenBank NCBI Reference Sequence NC_007779.1 (dated Jun. 7, 2020) position 1′881′835 to 1′882′473) of the O2a glycan-producing strain together with a selection marker (which was later removed) using known techniques (T E Kuhlman and E C Cox. Nucleic Acids Res. 2010 April; 38 (6): e92.), originating the pyruvylated O1v1 glycan-producing strain. The transcription of the inserted genes was driven by the K. pneumoniae promoters which are included in the inserted DNA and was constitutive. The KpO1v1 glycan produced in presence of wbbZ is here named KpO1v1 (Z+).

Gene wbbY and the DNA region upstream of it featuring a transcription promoter as in K. pneumoniae (GenBank Accession No. LT174607.1 (dated May 9, 2017) position 5′605 to 8′024) was used to replace the gene yeaS (GenBank NCBI Reference Sequence NC_007779.1 (dated Jun. 7, 2020) position 1′881′835 to 1′882′473) of the O2a glycan-producing strain together with a selection marker (which was later removed) using known techniques (T E Kuhlman and E C Cox. Nucleic Acids Res. 2010 April; 38 (6): e92.), originating the non-pyruvylated O1v1 glycan-producing strain. The transcription of the inserted genes was driven by the K. pneumoniae promoters which are included in the inserted DNA and was constitutive. The KpO1v1 glycan produced in absence of wbbZ is here named KpO1v1 (Z−).

Genes manC, manB, wzm, wzt, wbdD, wbdA, wbdB, wbdC as in K. pneumoniae (GenBank Accession No. LT174604.1 (dated Jun. 13, 2016)) were inserted into the 016 O-antigen cluster of the “clean strain” together with a selection marker (which was later removed) using known techniques (T E Kuhlman and E C Cox. Nucleic Acids Res. 2010 April; 38 (6): e92.), originating the O3b glycan-producing strain. The transcription of the inserted genes was driven by the native E. coli O-antigen cluster promoter and was therefore constitutive.

The five strains were transformed with plasmids encoding the inducible expression of the oligosaccharyl transferase PglB, the carrier protein EPA (detoxified exotoxin A from Pseudomonas aeruginosa) containing four PglB glycosylation consensus sequences, and, for O3b, a further copy of the genes manC and manB, generating the respective conjugate-producing strains. The expression of these genes was inducibly expressed by isopropyl β-D-1-thiogalactopyranoside (IPTG). The used plasmids vary among the four strain due to their specific better performance in terms of bioconjugate production. The amino acid sequences of the introduced EPA (e.g. SEQ ID NO: 18) and PglB proteins (e.g. SEQ ID NO: 15) are nevertheless identical among the four strains.

### Expression of the Bioconjugates

The ability of the five strains in producing the wanted bioconjugates was assessed in protein glycosylation experiments. The experiments consist in inoculating a liquid TBdev medium culture containing the appropriate antibiotics with the conjugate-production strain, incubating it in the optimal identified temperature until optimal OD, inducing the plasmid-encoded genes with optimal Ara and/or IPTG concentration, further incubate it until the optimal harvesting time, where the optimal parameters were identified after screening several alternatives in previous experiments. Such experiments are carried out earlier in shaking flasks and later in fed-batch bioreactors. The conjugate production was assessed by extracting the periplasm's content and analysing it on SDS page which was either stained with coomassie staining or transferred on blotting membranes for the execution of Western Blot analyses.

In FIG. 1 are reported analyses of conjugates extracted from Research-level shaking flasks experiment where EPA carrier with different numbers of PglB consensus glycosylation sequences were compared. The indicated glycan-producing strains were transformed with plasmids carrying an EPA variant and a plasmid expressing PglB. To prepare a pre-culture, 5 ml TB (Terrific Broth) medium containing 10 mM MgCl₂ and appropriate antibiotics was inoculated with a streak of colonies from the transformation plate and grown at 37° C. o/n (overnight). The pre-culture was used to inoculate 50 ml of supplemented TB medium in a shake flask to give a starting OD600=0.1. The cultures were grown at 37° C., with 200 rpm shaking until reaching OD600=0.8-1 and then induced by addition of 0.1 mM IPTG (PgIB). The expression and glycosylation of EPA variants was continued at 37° C. o/n.

A periplasmic extraction procedure was carried out. The amount of cells from o/n cultures corresponding to OD600=60 (measured using a spectrophotometer) was harvested by centrifugation. The cell pellets were resuspended in 1.5 ml of lysis buffer (30 mM Tris-HCl pH 8.5, 1 mM EDTA (Ethylenediaminetetraacetic acid), 20% sucrose) and lysozyme was added to a final concentration of 1 mg/ml. The suspensions were incubated with slight shaking for 25 minutes at 4° C. and then centrifuged at 16′000 rcf for 10 min. After centrifugation, the supernatant corresponding to periplasmic extract (PPE) was transferred to a fresh tube. Samples were detected on the gel by Coomassie staining (Fazekas de St. Groth, S.; Webster, R. G.; Datyner, A. (1963). “Two new staining procedures for quantitative estimation of proteins on electrophoretic strips”. Biochimica et Biophysica Acta. 71:377-391. doi: 10.1016/0006-3002 (63) 91092-8. PMID 18421828).

In order to enrich periplasmic extracts with EPA variants and allow more direct read-out by SDS-PAGE, the His-tagged EPA variants were purified using one-step purification on Ni-NTA (Nickel Nitrilo-triacetic Acid) agarose. 1 ml of PPE was mixed with 200 μl of pre-equilibrated Ni-NTA slurry and incubated with slight shaking for 30 min. After that the resin was washed and the bound protein eluted with elution buffer (30 mM Tris pH 8.0, 500 mM imidazole, 50 mM NaCl). The IMAC enriched PPE was analysed by SDS-PAGE (Laemmli, U. K. (1970). “Cleavage of Structural Proteins during the Assembly of the Head of Bacteriophage T4”. Nature. 227 (5259): 680-685. Bibcode: 1970Natur.227 . . . 680L. doi: 10.1038/227680a0. ISSN 0028-0836. PMID 5432063). Samples were detected on the gel by Coomassie staining (Fazekas de St. Groth, S.; Webster, R. G.; Datyner, A. (1963). “Two new staining procedures for quantitative estimation of proteins on electrophoretic strips”. Biochimica et Biophysica Acta. 71:377-391. doi: 10.1016/0006-3002 (63) 91092-8. PMID 18421828).

The bioreactor testing of the conjugate-producing strains was carried out as follows. pH 7 phosphate-buffered TBdev medium with 50 g/L glycerol, 10 mM MgCl2, antibiotics, was inoculated with the appropriate strain and stirred at 37° C. (or 35° C. for O2a) in a bioreactor vessel. Temperature was shifted to 30° C. (or kept at 37° C. for O3b) ahead of induction. Induction was carried out with 0.1 mM IPTG, and a feed was started at OD 25-40. Feed medium was phosphate-buffered at pH 7 and consists of yeast extract 67 g/L, Soy peptone 33 g/L, glycerol 250 to 300 g/L, 0.1 mM IPTG, antibiotics. Cells were harvested at 42-46 h after induction (or at 22-26 h for O3b). Samples for analysis were withdrawn at harvest.

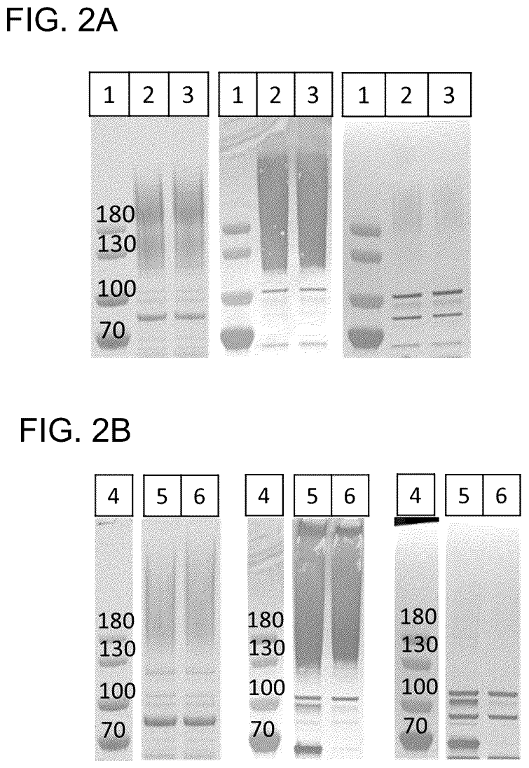

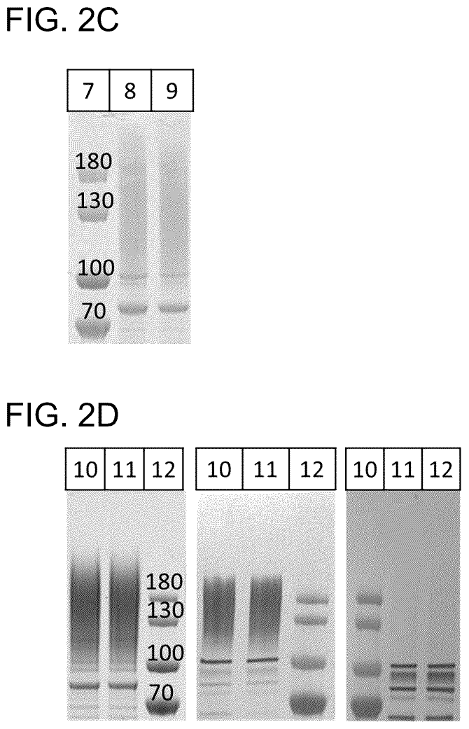

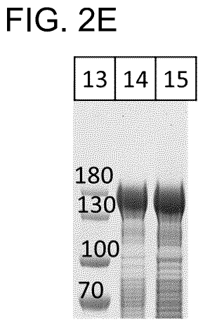

A periplasmic extraction procedure was carried out, followed by SDS-PAGE and Coomassie staining. Periplasmic extracts were also analysed by immunoblots using anti-serum raised against K. pneumoniae killed whole cells exposing the O-antigen of interest (FIG. 2). In FIG. 2 KpO1v1 (Z+) (panel A) and KpO1v1 (Z−) (panel B) are compared, showing the higher glycan length achieved with the exclusion of wbbZ.

### Purified Bioconjugates

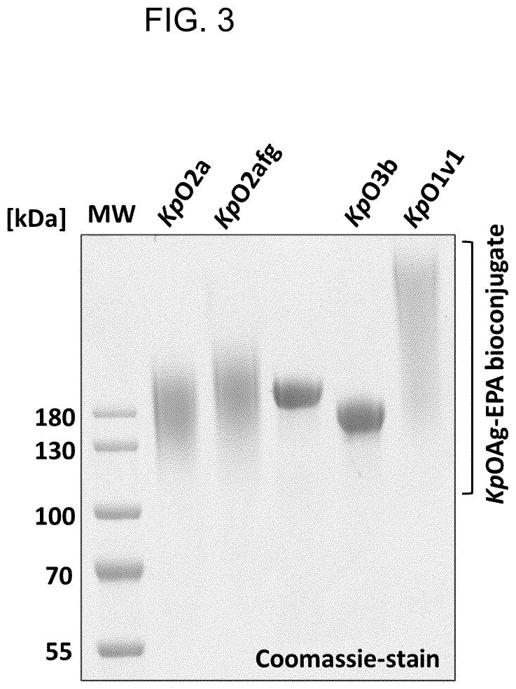

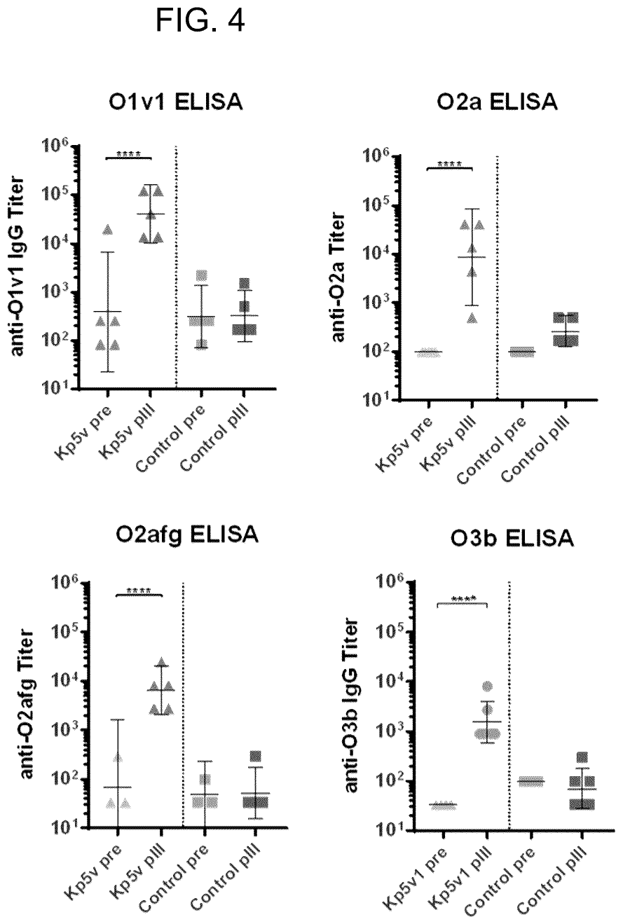

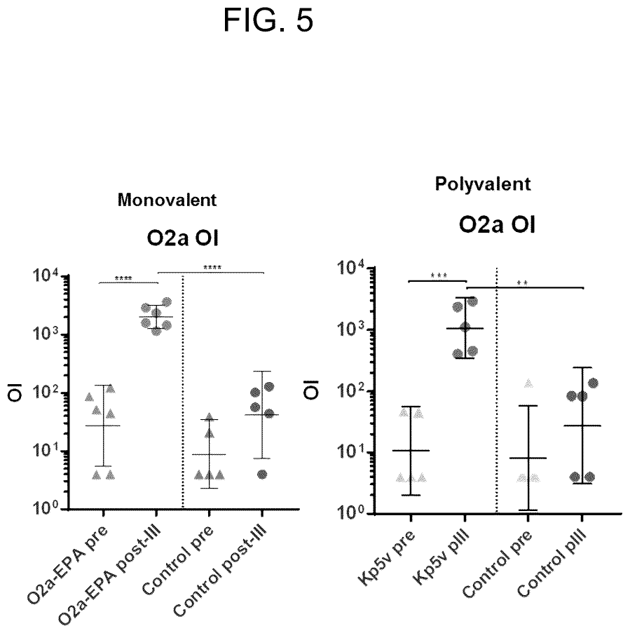

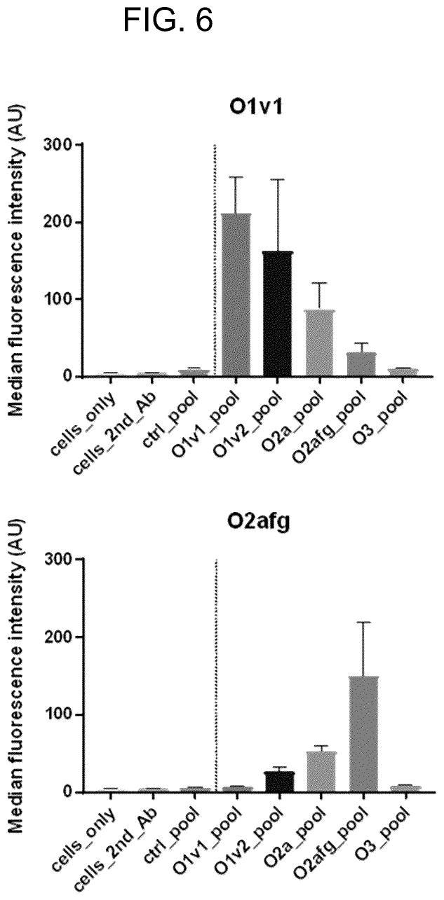

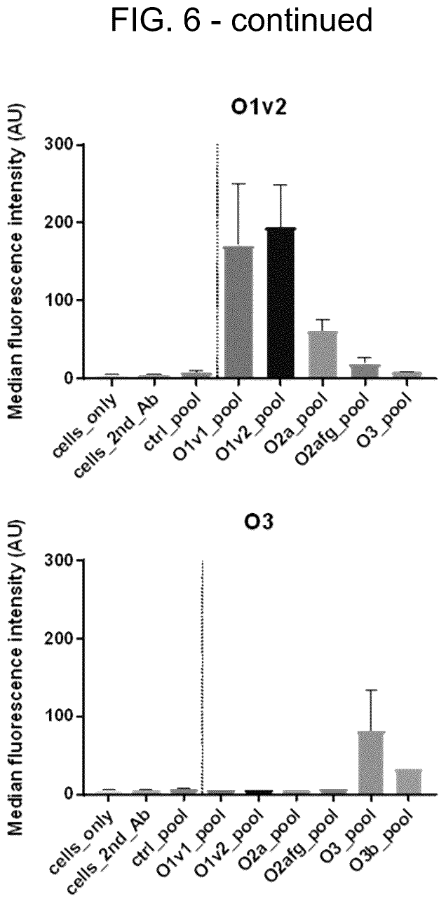

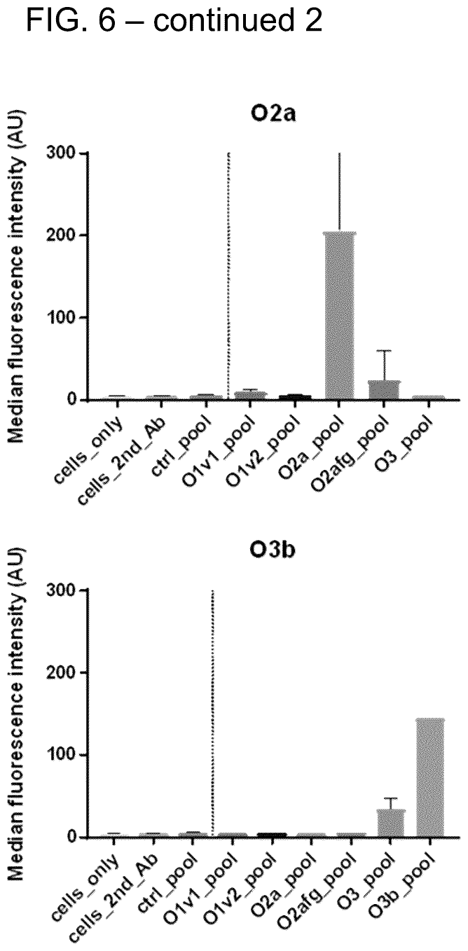

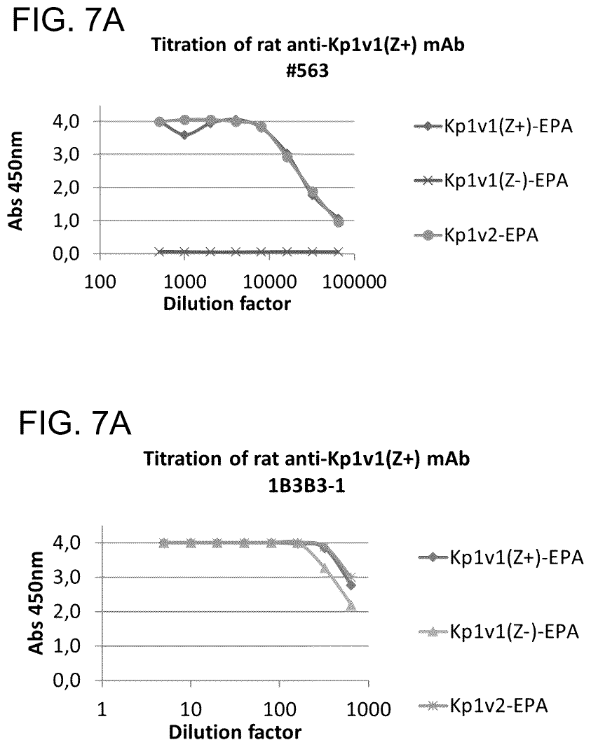

Periplasmic extraction was applied to the totality of the material harvested at the end of the growth protocol and the extracted solution was loaded into a series of chromatographic columns in order to separate contaminants and obtain a pure conjugate (FIG. 3).

### Example 2: Genetic Manipulation of K. pneumoniae Wild Type Strains for O-Antigen Characterization

The O-antigen is a part of the lipopolysaccharide (LPS). The cluster encoding the K-antigen (capsular polysaccharide) of K. pneumoniae isolates National Collection of Type Cultures (NCTC) Numbers: NCTC 13439, NCTC 9147, NCTC 11682, and NCTC 9163, expressing O-antigens O3b, O2afg, O1v1, and O2a, respectively was replaced by a kanamycin resistance cassette via homologous recombination as described (Datsenko, A. and Wanner, L. 2000, PNAS, 97 (12) 6640-6645) in order to minimize the likelihood of co-purification of the K-antigen together with the LPS. Fed-batch bioreactor cultivation was carried out for the obtained strains in order to maximize the biomass production. Cells were harvested and the LPS was extracted as described in Apicella M. A. 2008, Methods in Molecular Biology, 431:3-13 and a follow-up size exclusion chromatography was applied as described in Perdomo R. and Montero V. 2006, Biotecnología Aplicada 23:124-129.

In order to further understand the function of WbbZ in O1v1, the gene wbbZ was replaced by a XXX resistance cassette in the derivative of strain NCTC 11682 where the K-antigen was removed. In order to be consistent with the nomenclature given to the glycans produced recombinantly in E. coli described above, the O-antigen produced in strain where the wbbZ is still present is here named O1v1 (Z+), while the one produced in strain where wbbZ was replaced is here named O1v1(Z−).

### Example 3: Characterization of the Effect of wbbZ Deletion in K. pneumoniae Wild Type Strains

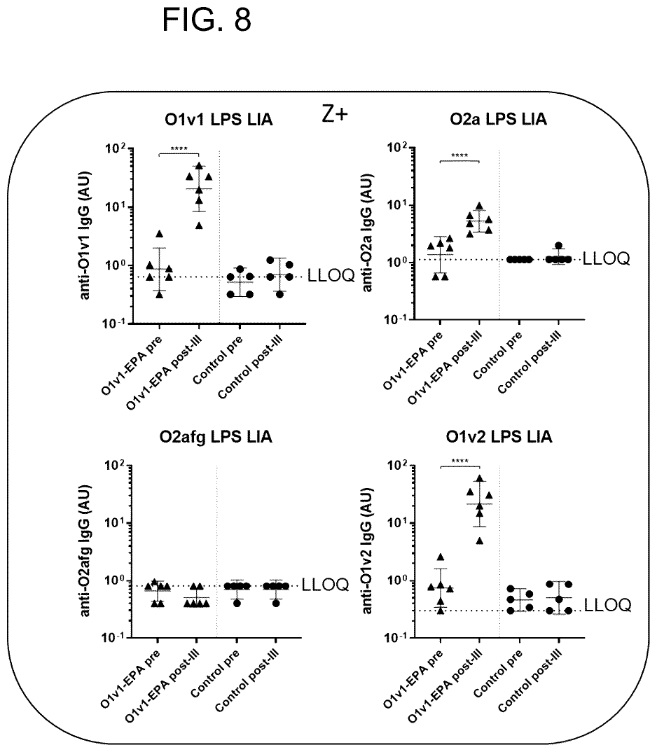

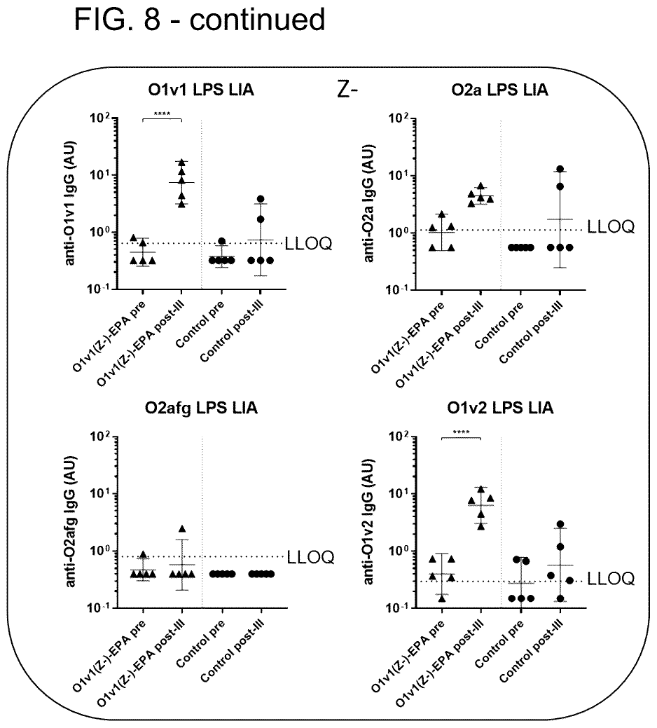

The LPS produced by K. pneumoniae NCTC 11682 derivatives lacking the K-cluster and where wbbZ was either intact or replaced by a selection marker, were analysed by silver staining and Western blots (FIG. 8). It is possible to observe that the LPS in strains lacking wbbZ bears an O-antigen with a broader distribution of lengths, and where the longer glycans are significantly longer when compared to the strain in which wbbZ is intact. Western blots where the monoclonal antibodies #563 and #576 were used show that #563, which proposedly recognized only the pyruvylate cap on galactan II, shows signal only in LPS from strain in which wbbZ is intact, while #576, proposedly binding the side chain of galactan II, does not discriminate the O-antigen from the two strains.

### Example 4: Structural Comparisons Between K. pneumoniae Natural O-Antigens and Glycan Part of Recombinantly Produced Bioconjugates

NMR Analyses of LPS from K. pneumoniae Wild Type Strains

The O-antigen is a part of the lipopolysaccharide (LPS). The cluster encoding the K-antigen (capsular polysaccharide) of K. pneumoniae isolates National Collection of Type Cultures (NCTC) Numbers: NCTC 13439, NCTC 9147, NCTC 11682, and NCTC 9163, expressing O-antigens O3b, O2afg, O1v1, and O2a, respectively was replaced by a kanamycin resistance cassette via homologous recombination as described (Datsenko, A. and Wanner, L. 2000, PNAS, 97 (12) 6640-6645) in order to minimize the likelihood of co-purification of the K-antigen together with the LPS. Fed-batch bioreactor cultivation was carried out for the obtained strains in order to maximize the biomass production. Cells were harvested and the LPS was extracted as described in Apicella M. A. 2008, Methods in Molecular Biology, 431:3-13 and a follow-up size exclusion chromatography was applied as described in Perdomo R. and Montero V. 2006, Biotecnología Aplicada 23:124-129.

Samples were prepared for NMR as follows. 80 mg LPS was suspended in 2 mL of 2% v/v acetic acid and hydrolyzed at 100° C. until precipitate formed. After removal of the precipitate by centrifugation and washing the pellet in 2% acetic acid, the pooled supernatant was subjected to size exclusion chromatography. Polysaccharide was separated on a Sephadex G-50 superfine column and fractions corresponding to the early peak (major) were pooled, evaporated to reduce the volume, and lyophilized. Dried polysaccharide was deuterium-exchanged by lyophilizing twice from 99.9% D₂O. For the NMR measurements polysaccharide was dissolved in 560 μL 99.9% D₂O and 4 μL 1% TSP in D₂O was added. The sample was centrifuged at 4,600×g for 5 min and placed into 5 mm NMR tube. 1H NMR and ¹H, ¹³C HSQC experiments were obtained using a Bruker Avance III 600 MHz spectrometer equipped with a 5 mm TXI probe. ¹³C NMR spectrum was obtained using a Bruker Avance III 400 MHz spectrometer equipped with a 5 mm broadband cryoprobe Prodigy. TSP was used as a chemical shift reference in the ¹H and ¹³C dimensions (δ_{H}=0 ppm, δ_{C}=−1.6 ppm). ¹H NMR spectrum was recorded at 30° C. and 50° C. 13C NMR and HSQC were recorded at 30° C. Results are summarized in Table 1.

### NMR Analyses of the Purified Conjugates

The O1v1(Z−)-EPA conjugate sample was exchanged twice with D₂O and then dissolved in 0.6 mL D₂O and transferred to a 5 mm NMR tube. NMR spectra were recorded at 323K. 1D (1H & DOSY) and 2D, TOCSY and HSQC-DEPT NMR spectra were obtained using a Bruker Avance III 600 MHz NMR spectrometer equipped with a BBO Prodigy cryoprobe. The spectra were recorded and processed using standard Bruker software (Topspin 3.2). The 1D proton spectra were recorded using a 30 degree pulse and a D1 of 5s. The 2D DOSY-TOCSY experiments was performed using a mixing time of 180 ms. The ¹H-¹³C HSQC experiment was optimized for J=145 Hz. 2D experiments were recorded using non-uniform sampling: 50% for homonuclear and 20% for heteronuclear experiments. Spectra were referenced relative to β-Galf: ¹H at 5.21 ppm, ¹³C at 110.2 ppm [Vinogradov et al. Structures of Lipopolysaccharides from Klebsiella pneumoniae, JBC, 2002, 277, 25070-25081].

The O1v1 (Z+)-EPA conjugate sample was exchanged twice with D₂O and then dissolved in 0.6 mL D₂O and transferred to a 5 mm NMR tube. NMR spectra were recorded at 323K. 1D (¹H and ³¹P) and 2D, COSY, TOCSY, NOESY and HSQC-DEPT NMR spectra were obtained using a Bruker Avance III 600 MHz NMR spectrometer equipped with a BBO Prodigy cryoprobe. The spectra were recorded and processed using standard Bruker software (Topspin 3.2). The 1D proton spectra were recorded using a 30 degree pulse and a D1 of 5s. 2D DOSY-TOCSY experiments were performed using a mixing time of 180 ms and NOESY for 300 ms. The ¹H-¹³C HSQC experiment was optimized for J=145 H. 2D experiments were recorded using non-uniform sampling: 50% for homonuclear and 25% for heteronuclear experiments. 1D and 1D TOCSY (200 ms) were also recorded at 323K. Spectra were referenced relative to β-Galf: ¹H at 5.21 ppm, ¹³C at 110.2 ppm [Clarke et al. “Molecular basis for the structural diversity in serogroup O2-antigen polysaccharides in Klebsiella pneumoniae.” Journal of Biological Chemistry 293.13 (2018): 4666-4679].

The O2a-EPA conjugate sample was exchanged twice with D₂O and then dissolved in 0.6 mL D₂O and transferred to a 5 mm NMR tube. NMR spectra were recorded at 323K. 1D (1H) and 2D, DOSY-TOCSY and HSQC-DEPT NMR spectra were obtained using a Bruker Avance III 600 MHz NMR spectrometer equipped with a BBO Prodigy cryoprobe. The spectra were recorded and processed using standard Bruker software (Topspin 3.2). The 1D proton spectra were recorded using a 30 degree pulse and a D1 of 5s. 2D DOSY-TOCSY experiments were performed using a mixing time of 180 ms, the ¹H-¹³C HSQC experiment was optimized for J=145 Hz. 2D experiments were recorded using non-uniform sampling: 50% for homonuclear and 25% for heteronuclear experiments. Spectra were referenced relative to β-Galf: ¹H at 5.22 ppm, ¹³C at 110.6 ppm [Clarke et al. “Molecular basis for the structural diversity in serogroup 02-antigen polysaccharides in Klebsiella pneumoniae.” Journal of Biological Chemistry 293.13 (2018): 4666-4679].

The O2afg-EPA conjugate sample was exchanged twice with D₂O and then dissolved in 0.6 mL D₂O and transferred to a 5 mm NMR tube. NMR spectra were recorded at 323K. 1D (1H). DOSY and 2D, DOSY-TOCSY and HSQC-DEPT NMR spectra were obtained using a Bruker Avance III 600 MHz NMR spectrometer equipped with a BBO Prodigy cryoprobe. The spectra were recorded and processed using standard Bruker software (Topspin 3.2). The 1D proton spectra were recorded using a 30 degree pulse and a D1 of 5s. The 2D DOSY-TOCSY experiment was performed using a mixing time of 180 ms; the ¹H-¹³C HSQC experiment was optimized for J=145 Hz. 2D experiments were recorded using non-uniform sampling: 50% for homonuclear and 20% for heteronuclear experiments. Spectra were referenced relative to b-Galf: ¹H at 5.22 ppm, ¹³C at 110.9 ppm [Clarke et al. “Molecular basis for the structural diversity in serogroup 02-antigen polysaccharides in Klebsiella pneumoniae.” Journal of Biological Chemistry 293.13 (2018): 4666-4679].

The O3b-EPA conjugate sample was exchanged twice with D₂O then dissolved in 0.6 mL D₂O and transferred to a 5 mm NMR tube for analysis. NMR spectra were recorded at 323K. 1D (¹H and DOSY and ³¹P) and 2D, COSY, DOSY-TOCSY, NOESY, HSQC-DEPT and ¹H-³¹P HMBC NMR spectra were obtained using a Bruker Avance III 600 MHz NMR spectrometer equipped with a BBO Prodigy cryoprobe. The spectra were recorded and processed using standard Bruker software (Topspin 3.2). The 1D proton spectra were recorded using a 30 degree pulse and a D1 of 5s. The 2D DOSY-TOCSY experiment were performed using mixing time of 180 ms (1D using 200 ms) and the 2D NOESY recorded using a mixing time of 300 ms. The ¹H-¹³C HSQC-DEPT experiment was optimized for J=145 Hz and the ¹H-³¹P HMBC experiment for J=50 Hz. Spectra were referenced relative to H1/C1 of 2-α-Man: ¹H at 5.36 ppm, ¹³C at 101.4 ppm and ³¹P at 2.08 ppm (Scientific reports 2017, 7, 6635). Results are summarized in Table 1.

TABLE 1
Comparison of relevant parameter determined by NMR studies
on wild type K. pneumoniae LPS and on purified glycoconjugates.

| | | | Degree of | | Ratio |
|---|---|---|---|---|---|
| | | | polymerization | Degree | gal-II |
| | | | (average repeat | of Gal | vs |
| Serotype | Source | Structure | units) | branching¹ | gal-I |
| O1v1 | LPS | Confirmed | N/P | N/A | 60:40 |
| | Conjugate | Confirmed | Gal-II: 6.1 | N/A | 62:38 |
| | | | Gal-I: 3.8 | | |
| O2a | LPS | Confirmed | 34 | N/A | N/A |
| | Conjugate | Confirmed | 17 | N/A | N/A |
| O2afg | LPS | Confirmed | N/P | 93% | N/A |
| | Conjugate | Confirmed | 7 | 100% | N/A |
| O3b | LPS | Confirmed | 12 | N/A | N/A |
| | Conjugate | Confirmed | 12 | N/A | N/A |

¹Percentage of Gal-III on Gal-I + Gal-III.
N/A = Not Applicable.
N/P = Not Possible.

### Example 5: Animal Studies on the Conjugates: Immunogenicity of the Conjugates, Functionality and Cross Reactivity of the Generated Antisera

### Immunogenicity and Cross Reactivity of KpO1v1-EPA Conjugate in Rabbits: Assessing the Effect of the Pyruvlyation

The immunogenicity of the purified conjugates has been assessed in rabbit immunization studies. Monovalent compositions of O1v1-EPA conjugates either with or without pyruvylation were injected. In general, groups of 5 or 6 New Zealand rabbits were immunized with monovalent KpO1v1(Z−)-EPA (non-pyruvylated form) or KpO1v1 (Z+)-EPA (pyruvylated form) compositions in 10 mM Na-phosphate pH 6.5, 150 mM NaCl buffer without adjuvants. Buffer only was used as control. 1 μg of total polysacchide was used for each injection. Three immunizations were carried out at day 0, 14, and 28 of the protocol. Pre-immunization, Post-II and Post-III bleeds were harvested at day 0, 28, and 42 of the protocol, respectively, and sera were obtained. The specific antibody content of each serum was measured via Luminex immunoassay (LIA). For magnetic bead coupling to LPS, methods previously described for pneumococcal capsular polysaccharides and Shigella LPS were used (Pickering et al 2002, Kaminski et al 2013). In brief, Kp LPS was added to 0.01% NaOH 0.0001% Phenolphthalein (PPT) and vortexed, cyanuric chloride was diluted to 50 mg/ml and added to a final concentration of 0.5 mg/ml, the solution was vortexed until colourless. Poly-L-lysine hydrobromide (PLL) was added to a final concentration of 5 μg per mg of LPS and the solution was incubated at 4° C. overnight. The solution was passed through a Sephadex G-25 column. MagPlex magnetic microspheres (BioRad) were vortexed and sonicated, washed in 50 mM HEPES buffer and pelleted using DynaMag-2 Magnetic Particle Concentrator (Invitrogen). Beads were activated with 5 mg/ml Sulpho-NHS and 5 mg/ml EDC (Thermo Fisher) on a Rotamix for 20 minutes at room temperature. Beads were washed and LPS-PLL was added in 50 mM HEPES buffer at various concentrations, vortexed and placed on Rotamix for 120 minutes at room temperature. Beads were counted using a haemocytometer, resuspended at 1×10⁷/ml and stored at 4° C. in PBS containing 0.1% BSA, 0.05% sodium azide, protected from light. LPS coupled beads were diluted and combined in 6-plex (2000 each serotype bead per well) in assay buffer containing 1% BSA, 0.05% Tween 20. Beads were incubated with diluted sera, standard or quality control sample (assay buffer alone was used for the blank wells), the plate was sealed and incubated with shaking at 500 rpm at room temperature for 1 hour. Plates were washed 2× using BioRad Bio-Plex Pro Wash Station, R-phycoerythrin conjugated goat anti-rabbit IgG detection antibody (Sigma) at 3 μg/ml was added to each well and sealed and incubated with shaking at 500 rpm at room temperature for 30 minutes. Plates were washed 3× and beads were resuspended in assay buffer and analysed on Luminex 200 on high PMT setting. Serum antibody responses against LPS were monitored pre, post second and post-third immunization (pre, post-II and post-III). The immunogenicity of the test items was assessed by measuring LPS-specific IgG serum levels. MFI values above the LLOQ were interpolated using a calibration curve to an arbitrary concentration, values below the LLOQ were assigned a value of ½ LLOQ to be able to analyze results.

In FIG. 8 the results are reported (Post-II omitted). It is possible to observe that both versions of the KpO1v1-EPA conjugates are able to elicit the biosynthesis of antibodies which react with O1v1 and O1v2. The omission of the pyruvylation is therefore not compromising the immunogenicity towards these serotypes.

### Immunogenicity of Tetravalent Composition in Rabbits

A multivalent composition composed of KpO1v1(Z−)-EPA, KpO2a-EPA, KpO2afg-EPA, KpO3b-EPA, named Kleb4V has been used for a rabbits immunization study. Groups of 5 New Zealand rabbit were immunized with Kleb4V in absence of adjuvant, in presence of AS03 adjuvant in 10 mM Na-phosphate pH 6.5, 150 mM NaCl or with buffer only as control. 1 μg of polysacchide per serotype (total 4 μg polysaccharide) was used for each injection. Three immunizations were carried out at day 0, 14, and 28 of the protocol. Pre-immunization, Post-II and Post-III bleeds were harvested at day 0, 28, and 42 of the protocol, respectively, and sera were obtained. The specific antibody content of each serum was measured via Luminex immunoassay (LIA) as described in the previous paragraph.

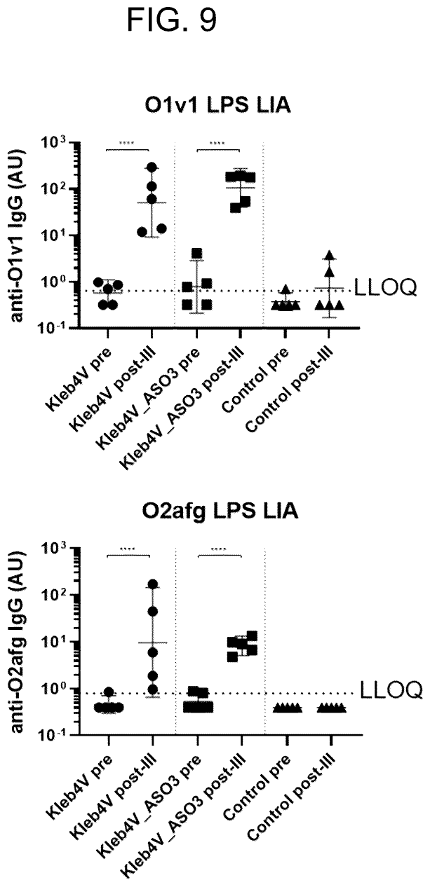

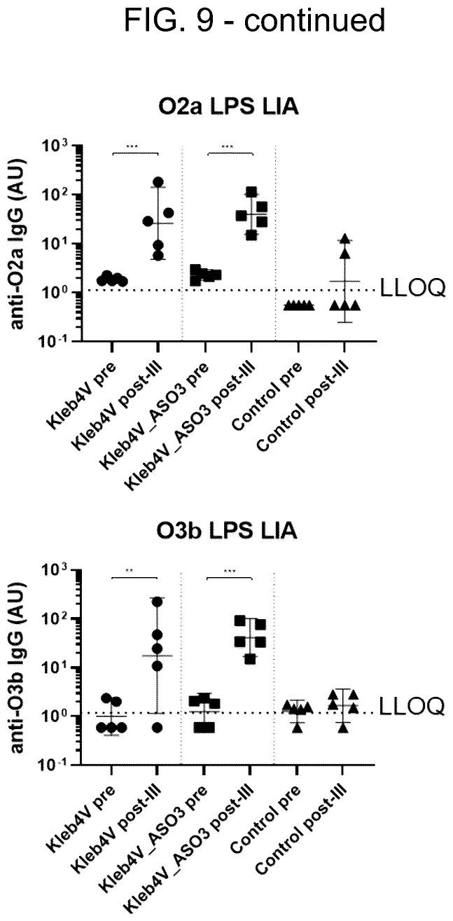

In FIG. 9 a summary of the results for polyvalent composition is reported for the conjugates of interest (Post-II omitted). Conjugates were able to significantly elicit the production of O-antigen specific antibodies against the four vaccine serotypes.

### Example 6: Serogroup O1 Structural Determination

The present inventors have found that the structural characterization of Klebsiella O-antigen serotypes belonging to 01 serogroup reported in publicly available literature is incomplete. The present inventors have created O1v1 conjugates in strains either including or excluding the gene wbbZ in the E. coli production strains. Certain monoclonal antibodies originated in rats immunized with the conjugate produced from an E. coli production strain containing wbbZ were unable to recognize a conjugate produced from an E. coli O1v1-producing strain devoid of wbbZ, while they were able to recognize conjugates produced from E. coli O1v1 and O1v2 producing strains containing wbbZ, as well as the klebsiella LPS from 01 serogroup. Deeper NMR analysis confirmed that a pyruvate linked to the 3 and 4 position of the non reducing terminal galactose can be detected in a conjugate produced by a strain which includes wbbZ but not in one which does not. Similarly the pyruvylation was detected in LPS extracted by klebsiella strains.

In order to prove the correlation between wbbZ and pyruvylation, the LPS from a Klebsiella pneumoniae O1v1 strain in which wbbZ is mutated was analyzed via Western Blot in comparison to the same strain in which wbbZ has been recombinantly introduced. Recombinant expression of wbbZ renders the LPS recognizable by the pyruvylation-specific antibody, having an effect in reducing the length of the O-antigen.

Moreover, the activity of wbbZ was investigated in vitro. Purified WbbZ protein, WbbY (the processive multi-functional glycosyltranferase responsible for the synthesis of the galactan II polysaccharide elongating the non-reducing end of galactan I), UDP-galactose, phosphoenolpyruvate, and a synthetic galactan I disaccharide linked to a fluorophore at the reducing end were differently mixed in the appropriate buffer and reactions were carried out. The running behavior of the fluorophore-linked glycan was then assessed via SDS-PAGE exploiting the fluorescence. Reactions in which WbbZ and/or phosphoenolpyruvate were omitted resulted in an omogeneously long glycan. The inclusion of both WbbZ and phosphoenolpyruvate created a glycan length distribution with several species of shorter length (FIG. 8) indicating i. the role of the WbbZ-mediated modification in shortening the O1 polysaccharide and ii. the function of WbbZ depends on a pyruvate donor. When a mutant version of WbbY able to add only a galactan II disaccharide to the galactan I acceptor was used in the reactions, the omission of WbbZ resulted in a sharp band corresponding to the generated tetrasaccharide (galactan II disaccharide-galactan I disaccharide-fluorofore). Addition of WbbZ and phosphoenolpyruvate resulted instead in a different shift which suggests that a negative charge was introduced. The glycan produced with this last reaction was analyzed by NMR, confirming that analysis confirmed the presence of a pyruvate linked to the 3 and 4 position of the non reducing terminal galactose. WbbZ structure is available (PDB: 6X1L) and it is similar to a characterized pyruvyltransferase from yeast (PDB: 5AX7). Potential catalytic residues were inferred based on the homology and point mutants of WbbZ were created. The employment of such mutants in the in vitro assay resulted in the same electrophoretic pattern of the reactions in which WbbZ was omitted, confirming that the mutated residues are necessary for WbbZ's activity. NMR analysis of the glycan produced by including WbbZ, phosphoenolpyruvate.

The exclusion of wbbZ creates a glycan which is different from the wild type. This allows the production of a conjugate with longer glycans, resulting in higher sugar-to-protein ratio and ultimately allowing to inject less EPA in order to achieve the same glycan dosage. An effect on immunogenicity of the pyruvate has been excluded in animal immunization studies, where high Ig titers against O1v1 LPS were observed upon immunization with both pyruvylated and non-pyruvylated KpO1v1 conjugates.

TABLE 2
Comparison of structures of 01v1(Z+) and 01v1(Z−)

| | | | Degree of | Degree of | | Sugar/ |
|---|---|---|---|---|---|---|
| | | | (average repeat | Gal | Ratio gal- | Protein |
| Serotype | Source | Structure | units) | branching | II vs gal-I | ratio* |
| O1v1(Z+) | LPS | Confirmed, | N/P | N/A | 60:40 (II:I) | N/A |
| | | contains | | | | |
| | | pyruvlyation | | | | |
| | Conjugate | Confirmed, | Gal-II: 6.1 | N/A | 62:38 (II:I) | 42% |
| | | contains | Gal-I: 3.8 | | | |
| | | pyruvylation | | | | |
| O1v1(Z−) | LPS | Confirmed, | N/A | N/A | N/A | N/A |
| | | lacks | | | | |
| | | pyruvylation | | | | |
| | Conjugate | Confirmed, | Gal-II: 12 | N/A | 79:21 (II:I) | 86% |
| | | lacks | Gal-I: 3.2 | | | |
| | | pyruvylation | | | | |

*From Research-scale purifications. Glycan content measured via HPAEC-PAD, protein content measured via absorbance.

The structures of O1v2(Z+) and O1v2(Z−) conjugate have also been studied. The use of Coomassie stained SDS-PAGE gels demonstrates that longer glycans are present in the absence of wbbZ (i.e. in the case of O1v2(Z−)). NMR analysis confirmed the lack of pyruvylation in the O1v2(Z−) O-antigen, whereas pyruvylation could be detected in O1v2(Z+) O-antigen (results not shown). It therefore appears that the omission of the wbbZ gene in host cells producing a O1v2 O-antigen results in a lack of pyruvyation, capping the saccharide, and hence to a longer glycan.

### Example 7

A Phase I/II clinical trial was performed in an observer-blind, controlled and randomised matter in two steps to evaluated the safety and immunogenicity of a Kleb4V vaccine +/−AS03 adjuvant. The Kleb4V vaccine contains O-antigens of Klebsiella pneumoniae of serotypes O1v1, O2a, O2afg and O3b linked to detoxified Pseudomonas aeruginosa Exoprotein A (EPA), as bioconjugates. In the first step the target population contained 18-40 year olds and 55-70 year olds and in the second step, the target population was 55-70 year olds. Each subject was immunised with the Kleb4V vaccine, either unadjuvanted or adjuvanted with an AS03 adjuvant and either at a target dose (64 μg total saccharide dose-16 μg of each serotype) or a low dose (16 μg total saccharide dose-4 μg of each serotype). A total of 166 subjects were included in the study, 48 in step 1 and 118 in step 2. Each subject received two doses of the vaccine intramuscularly, 2 months apart.

### Safety Results

| | | | Low dose + | Target | Target dose + |
|---|---|---|---|---|---|
| | Placebo | Low dose | AS03 | dose | AS03 |
| | N (%) | N (%) | N (%) | N (%) | N (%) |
| Any AEs | 23 (67.6%) | 18 (60%) | 27 (90%) | 33 (91.7%) | 36 (100%) |
| Any solicited | 14 (41.2%) | 18 (60%) | 27 (90%) | 30 (83.3%) | 36 (100%) |
| AEs | | | | | |
| Any solicited | 5 (14.7%) | 15 (50%) | 27 (90%) | 26 (72.2%) | 35 (97.2%) |
| local AEs | | | | | |
| Any solicited | 13 (38.2%) | 7 (23.3%) | 18 (60.0%) | 20 (55.6%) | 18 (50%) |
| general AEs | | | | | |
| Any unsolicited | 17 (50%) | 10 (33.3%) | 11 (36.7%) | 22 (61.1%) | 21 (58.3%) |
| AEs | | | | | |
| SAEs | 3 (8.8%) | 10 (33.3%) | 11 (36.7%) | 1 (2.8%) | 1 (2.8%) |

It is concluded that the Kleb4V is safe in the target population of adults aged 55-70 years old. The majority of AEs were of mild intensity. There was a trend towards an impact of dose and adjuvantation on the incidence of AEs. All SAEs were unrelated to treatment and are resolved.

### Immunogenicity Results

Diluted sera were assessed for the presence of antibodies binding to K. pneumoniae serotype O1v1, Olv2, O2a, O2agf and O3b using a Multoplexed-LUMINEX assay. Serum samples were diluted and pipetted un duplicate into microwells. Microspheres coated with the required K. pneumoniae O-antigen were added to the wells and incubated at room temperature with shaking. A detection antibody, R-Phycoerythrin—anti-IgG was added to each well with subsequent incubation at room temperature with shaking. Finally fluorescence was detected and antibody titres calculated.

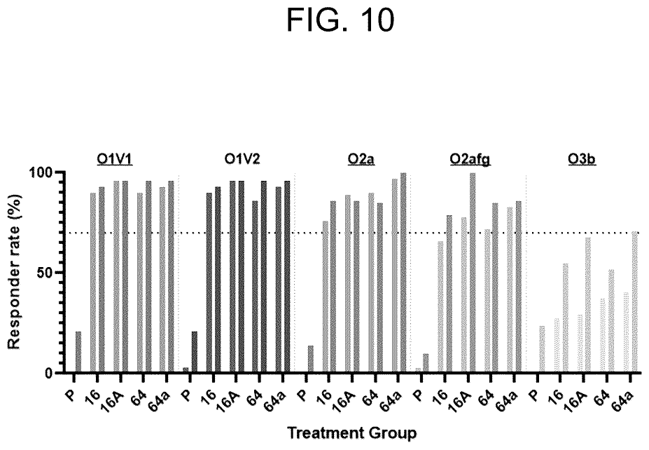

The results (see FIG. 10) showed over 70% seroconversion for all serotypes after 2nd vaccination. Indeed over 70% seroconversion was achieved after the first vaccination for all serotypes but O3b. The increase in responders rate following a second vaccination and with adjuvant was more relevant for O3b.

Functional assays were used to assess sera samples taken from the subjects. Dilutions of the sera were added to microwells in duplicate. The sera were inactivated by incubating at 56 degrees C. for 30 minutes. Pre-diluted bacteria were added to each well. Both SBA and OPA functional assays were carried out by adding complement (for SBA assays) or complement and HL-60 cells (for OPK assays) after incubation, each well was spotted onto an agar plate and after an overnight incubation, the number of CFUs was calculated.

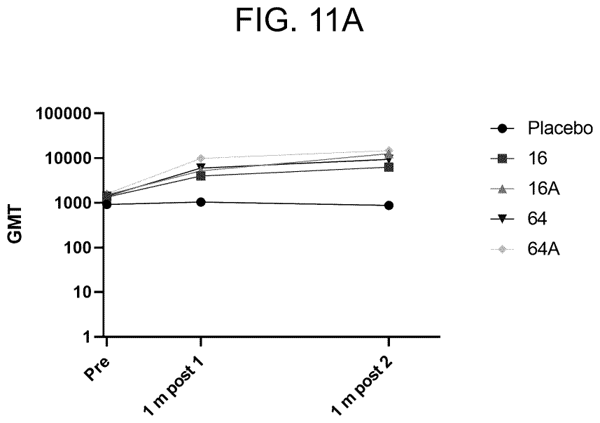

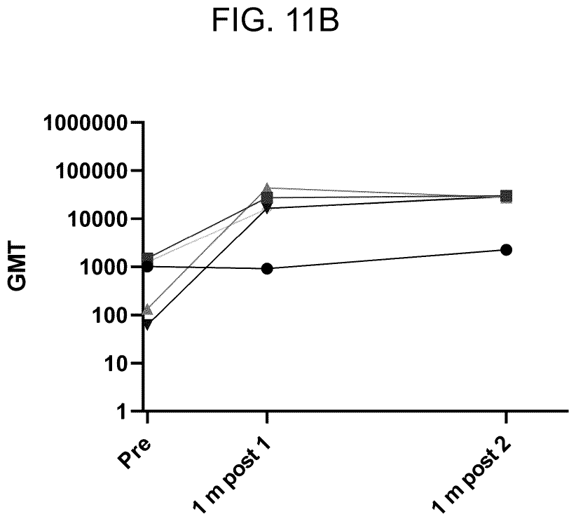

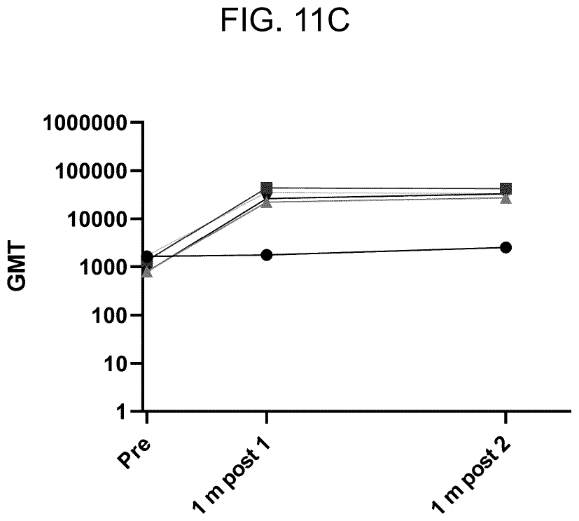

The results are shown in FIG. 11. Functional immune responses were monitored by mearuring SBA response to O2afg (FIG. 11A), as well as OPA responses to O1v1 (FIG. 11B) and O1v2 (FIG. 11C). Functional assays were not developed for serotypes O2a and O3b because of technical difficulties in identifying suitable strains. Significantly higher immune responses were noted in active vaccinees compared to baseline and placebo. O1v1 induced antibodies also protected against both O1v1 and O1v2. O2afg was also shown to elicit functional antibodies which neutralised O2afg in an SBA assay. Significant responses were detected in sera collected 1 month after the first immunisation and 1 month after the second immunisation.

### Sequences:

SEQ ID NO: 1
Consensus sequence (artificial sequence)
D/E-X-N-Z-S/T
SEQ ID NO: 2 Consensus sequence (artificial sequence)
K-D/E-X-N-Z-S/T-K
SEQ ID NO: 3
Consensus sequence (artificial sequence)
K-D-Q-N-A-T-K
SEQ ID NO: 4
Consensus sequence (artificial sequence)
J-D/E-X-N-Z-S/T-U
SEQ ID NO: 5
Consensus sequence (artificial)
G-S-G-G-G-D/E-X-N-Z-S/T-G-S-G-G
SEQ ID NO: 6
E. coli flagellin (FlgI) signal sequence
MIKFLSALILLLVTTAAQA
SEQ ID NO: 7
E. coli outer membrane porin A (OmpA) signal sequence
MKKTAIAIAVALAGFATVAQA
SEQ ID NO: 8
E. coli maltose binding protein (MalE) signal sequence
MKIKTGARILALSALTTMMFSASALA
SEQ ID NO: 9
Erwinia carotovorans pectate lyase (PelB) signal sequence
MKYLLPTAAAGLLLLAAQPAMA
SEQ ID NO: 10
heat labile E. coli enterotoxin LTIIb signal sequence
MSFKKIIKAFVIMAALVSVQAHA
SEQ ID NO: 11
Bacillus subtilis endoxylanase XynA signal sequence
MFKFKKKFLVGLTAAFMSISMFSATASA
SEQ ID NO: 12
E. coli DsbA signal sequence
MKKIWLALAGLVLAFSASA
SEQ ID NO: 13
E. coli TolB signal sequence
MKQALRVAFGFLILWASVLHA
SEQ ID NO: 14
Streptococcus agalactiae SipA signal sequence
MKMNKKVLLTSTMAASLLSVASVQAS
SEQ ID NO: 15
pgIB from Campylobacter jejuni
MLKKEYLKNPYLVLFAMIILAYVFSVFCRFYWVWWASEFNEYFFNNQLMIISNDG
YAFAEGARDMIAGFHQPNDLSYYGSSLSALTYWLYKITPFSFESIILYMSTFLSS
LVVIPTILLANEYKRPLMGFVAALLASIANSYYNRTMSGYYDTDMLVIVLPMFIL
FFMVRMILKKDFFSLIALPLFIGIYLWWYPSSYTLNVALIGLFLIYTLIFHRKEK
IFYIAVILSSLTLSNIAWFYQSAIIVILFALFALEQKRLNFMIIGILGSATLIFL
ILSGGVDPILYQLKFYIFRSDESANLTQGFMYFNVNQTIQEVENVDLSEFMRRIS
GSEIVFLFSLFGFVWLLRKHKSMIMALPILVLGFLALKGGLRFTIYSVPVMALGF
GFLLSEFKAIMVKKYSQLTSNVCIVFATILTLAPVFIHIYNYKAPTVFSQNEASL
LNQLKNIANREDYVVTWWDYGYPVRYYSDVKTLVDGGKHLGKDNFFPSFALSKDE
QAAANMARLSVEYTEKSFYAPQNDILKTDILQAMMKDYNQSNVDLFLASLSKPDF
KIDTPKTRDIYLYMPARMSLIFSTVASFSFINLDTGVLDKPFTFSTAYPLDVKNG
EIYLSNGVVLSDDFRSFKIGDNVVSVNSIVEINSIKQGEYKITPIDDKAQFYIFY
LKDSAIPYAQFILMDKTMFNSAYVQMFFLGNYDKNLFDLVINSRDAKVFKLKI
SEQ ID NO: 16
EPA sequence from Pseudomonas aeruginosa
AEEAFDLWNECAKACVLDLKDGVRSSRMSVDPAIADTNGQGVLHYSMVLEGGNDA
LKLAIDNALSITSDGLTIRLEGGVEPNKPVRYSYTRQARGSWSLNWLVPIGHEKP
SNIKVFIHELNAGNQLSHMSPIYTIEMGDELLAKLARDATFFVRAHESNEMQPTL
AISHAGVSVVMAQAQPRREKRWSEWASGKVLCLLDPLDGVYNYLAQQRCNLDDTW
EGKIYRVLAGNPAKHDLDIKPTVISHRLHFPEGGSLAALTAHQACHLPLEAFTRH
RQPRGWEQLEQCGYPVQRLVALYLAARLSWNQVDQVIRNALASPGSGGDLGEAIR
EQPEQARLALTLAAAESERFVRQGTGNDEAGAASADVVSLTCPVAAGECAGPADS
GDALLERNYPTGAEFLGDGGDVSFSTRGTQNWTVERLLQAHRQLEERGYVFVGYH
GTFLEAAQSIVFGGVRARSQDLDAIWRGFYIAGDPALAYGYAQDQEPDARGRIRN
GALLRVYVPRWSLPGFYRTGLTLAAPEAAGEVERLIGHPLPLRLDAITGPEEEGG
RVTILGWPLAERTVVIPSAIPTDPRNVGGDLDPSSIPDKEQAISALPDYASQPGK
PPREDLK
SEQ ID NO: 17
Modified EPA sequence with consensus sequences inserted at N-
terminal + Y208 + R274 + A519 (artificial sequence)
GSGGGDQNATGSGGGKLAEEAFDLWNECAKACVLDLKDGVRSSRMSVDPAIADTN
GQGVLHYSMVLEGGNDALKLAIDNALSITSDGLTIRLEGGVEPNKPVRYSYTRQA
RGSWSLNWLVPIGHEKPSNIKVFIHELNAGNQLSHMSPIYTIEMGDELLAKLARD
ATFFVRAHESNEMQPTLAISHAGVSVVMAQAQPRREKRWSEWASGKVLCLLDPLD
GVYNKDQNATKLAQQRCNLDDTWEGKIYRVLAGNPAKHDLDIKPTVISHRLHFPE
GGSLAALTAHQACHLPLEAFTKDQNATKHRQPRGWEQLEQCGYPVQRLVALYLAA
RLSWNQVDQVIRNALASPGSGGDLGEAIREQPEQARLALTLAAAESERFVRQGTG
NDEAGAASADVVSLTCPVAAGECAGPADSGDALLERNYPTGAEFLGDGGDVSFST
RGTQNWTVERLLQAHRQLEERGYVFVGYHGTFLEAAQSIVFGGVRARSQDLDAIW
RGFYIAGDPALAYGYAQDQEPDARGRIRNGALLRVYVPRWSLPGFYRTGLTLKDQ
NATKAPEAAGEVERLIGHPLPLRLDAITGPEEEGGRVTILGWPLAERTVVIPSAI
PTDPRNVGGDLDPSSIPDKEQAISALPDYASQPGKPPREDLK
SEQ ID NO: 18
Modified EPA sequence with consensus sequences inserted at N-
terminal + Y208 + R274 + A519 and E. coli DsbA signal
sequence (artificial sequence)
MKKIWLALAGLVLAFSASAGSGGGDQNATGSGGGKLAEEAFDLWNECAKACVLDL
KDGVRSSRMSVDPAIADTNGQGVLHYSMVLEGGNDALKLAIDNALSITSDGLTIR
LEGGVEPNKPVRYSYTRQARGSWSLNWLVPIGHEKPSNIKVFIHELNAGNQLSHM
SPIYTIEMGDELLAKLARDATFFVRAHESNEMQPTLAISHAGVSVVMAQAQPRRE
KRWSEWASGKVLCLLDPLDGVYNKDQNATKLAQQRCNLDDTWEGKIYRVLAGNPA
KHDLDIKPTVISHRLHFPEGGSLAALTAHQACHLPLEAFTKDQNATKHRQPRGWE
QLEQCGYPVQRLVALYLAARLSWNQVDQVIRNALASPGSGGDLGEAIREQPEQAR
LALTLAAAESERFVRQGTGNDEAGAASADVVSLTCPVAAGECAGPADSGDALLER
NYPTGAEFLGDGGDVSFSTRGTQNWTVERLLQAHRQLEERGYVFVGYHGTFLEAA
QSIVFGGVRARSQDLDAIWRGFYIAGDPALAYGYAQDQEPDARGRIRNGALLRVY
VPRWSLPGFYRTGLTLKDQNATKAPEAAGEVERLIGHPLPLRLDAITGPEEEGGR
VTILGWPLAERTVVIPSAIPTDPRNVGGDLDPSSIPDKEQAISALPDYASQPGKP
PREDLK
SEQ ID NO. 19 Forward primer (artificial sequence)
AAGCTAGCGCCGCCGAGGAAGCCTTCGACC
SEQ ID NO. 20 Reverse primer (artificial sequence)
AAGAATTCTCAGTGGTGGTGGTGGTGGTGCTTCAGGTCCTCGCGCGGCGG
SEQ ID NO: 21 Klebsiella pneumoniae Wzm:
ATGAAGTACAATTTAGGGTATTTATTTGATTTACTTGTTGTCATAACAAATAAAG
ATCTAAAAGTGCGCTATAAGAGCAGCATGCTAGGCTATTTATGGTCAGTAGCAAA
TCCATTGCTTTTTGCCATGATTTATTATTTTATATTTAAGCTGGTAATGAGAGTA
CAAATTCCAAATTATACCGTTTTCCTCATTACCGGCTTGTTTCCGTGGCAATGGT
TTGCCAGTTCGGCCACTAACTCATTATTTTCATTCATCGCTAACGCTCAAATTAT
CAAGAAGACAGTTTTTCCCCGGTCCGTGATTCCGCTAAGTAATGTAATGATGGAA
GGGTTGCATTTTCTTTGTACCATCCCGGTTATTGTTGTCTTTCTTTTTGTTTATG
GCATGACGCCGTCCTTGTCCTGGGTTTGGGGTATACCTCTCATTGCTATTGGCCA
GGTGATTTTCACCTTTGGTGTTTCAATCATCTTTTCAACGCTGAACCTGTTTTTC
CGTGACCTGGAGCGCTTTGTCAGTCTGGGGATTATGCTGATGTTTTATTGTACGC
CGATTTTATATGCGTCTGATATGATTCCGGAAAAATTTAGCTGGATAATTACCTA
CAATCCGCTAGCGAGTATGATTCTTAGTTGGCGTGATTTATTCATGAATGGGACT
CTTAATTATGAGTATATTTCTATACTCTATTTTACGGGAATCATTTTGACGGTTG
TCGGTTTGTCTATTTTCAATAAATTAAAATATCGATTTGCAGAGATCTTGTAA
SEQ ID NO: 22 Klebsiella pneumoniae Wzt:
ATGCACCCAGTTATTAACTTCAGTCATGTTACAAAAGAGTATCCTCTGTACCATC
ATATTGGCTCAGGAATCAAAGATTTAATTTTCCATCCGAAACGCGCTTTTCAATT
GCTGAAGGGGCGGAAATATTTAGCTATCGAAGACGTATCCTTTACAGTTGGCAAA
GGTGAGGCTGTTGCTCTGATTGGACGTAATGGGGCAGGAAAGAGTACCTCTCTTG
GCCTGGTTGCCGGCGTGATTAAGCCAACTAAGGGAACCGTCACCACTGAAGGACG
GGTGGCATCGATGCTTGAACTCGGCGGAGGCTTTCATCCGGAACTTACCGGGCGT
GAGAATATTTACCTGAATGCTACTCTGCTGGGCCTTCGGCGTAAAGAGGTCCAGC
AACGTATGGAACGTATTATTGAATTTTCGGAACTGGGAGAATTCATAGACGAGCC
AATCAGAGTGTACTCAAGCGGAATGCTAGCTAAGTTAGGTTTTTCGGTCATCAGT
CAGGTTGAACCGGATATTTTAATTATTGATGAAGTTCTGGCAGTAGGTGATATCG
CTTTTCAGGCAAAATGTATTCAGACCATAAGAGATTTTAAGAAAAGAGGCGTGAC
AATATTGTTTGTTAGCCACAATATGAGTGACGTTGAAAAAATCTGCGACAGAGTC
ATCTGGATCGAAAATCATAGGCTCAGAGAAGTGGGGTCTGCAGAGCGAATCATTG
AACTGTACAAGCAAGCAATGGCTTAA
SEQ ID NO: 23
Klebsiella pneumoniae WbbM:
ATGAACAATAGCGTTAAAATCTATACCAGCCACCATAAGCCTAGTGCTTTTCTTA
ATGCTGCAATTATCAAACCTCTGCATGTCGGCAAAGCTAATTCTTGTAATGAAAT
TGGTTGTCCAGGAGATGACACTGGCGATAATATTTCCTTTAAGAATCCGTTTTAT
TGCGAACTTACTGCGCATTATTGGGTTTGGAAAAACGAAGAGCTGGCAGACTATG
TCGGTTTCATGCACTATCGCCGTCATCTTAATTTTTCCGAAAAACAAACTTTTTC
TGAGGATACGTGGGGGGTCGTGAACCATCCATGCATTGATGAAGAATATGAGGAG
ATCTTTGGATTAAACGAAGAAACAATTCAACGGTGTGTCGAAGGTATTGACATCT
TGCTGCCCAAAAAATGGTCTGTCACTGCGGCGGGAAGTAAAAATAATTACGATCA
CTATGAACGAGGTGAATACTTACACATTCGTGATTATCAGGCTGCCATTGCCACC
GTTGAAAAACTATATCCAGAGTATAGCACGGCAATAAAAACGTTTAATGATGCCA
GTGATGGCTATTACACAAATATGTTTGTCATGCGCAAAGATATTTTTGTTAACTA
TTCTGAGTGGCTCTTTTCTATTCTGGATAATCTCGAAGATGCCATCTCGATGAAC
AATTATAATGCTCAGGAAAAACGCGTTATTGGGCATATAGCAGAACGGCTGTTTA
ATATTTACATTATTAAGTTGCAACAAGATGGTGAGCTTAAGGTAAAAGAATTACA
GCGTACTTTTGTCAGCAATGAAACATTCAATGGTGCACTGAATCCAGTTTTTGAT
TCTGCGGTTCCAGTGGTTATCAGTTTCGATGATAATTACGCAGTCAGCGGTGGTG
CATTAATTAATTCCATTGTCCGGCATGCGGATAAAAATAAAAATTATGATATCGT
CGTGCTCGAAAACAAAGTAAGCTATTTGAATAAAACGCGGTTAGTAAATCTAACC
TCGGCTCATCCGAATATTTCTCTTCGTTTTTTTGACGTTAATGCCTTCACTGAAA
TAAACGGTGTGCATACCCGAGCGCATTTTAGCGCATCAACGTATGCCCGTCTTTT
TATTCCTCAACTGTTCAGACGATACGATAAAGTCGTATTTATTGATTCGGATACC
GTTGTAAAGGCTGACCTGGGTGAACTGCTTGATGTCCCTCTGGGCAACAATTTAG
TTGCAGCGGTTAAGGATATCGTCATGGAAGGTTTTGTAAAATTTTCTGCAATGTC
GGCATCAGATGATGGCGTTATGCCGGCAGGCGAATATTTACAGAAAACCTTAAAT
ATGAATAACCCTGATGAATATTTTCAGGCAGGGATTATTGTTTTTAATGTCAAAC
AAATGGTCGAAGAAAATACTTTTGCTGAATTGATGCGGGTATTAAAGGCAAAAAA
ATACTGGTTCCTCGACCAGGATATCATGAATAAAGTATTCTACTCTCGAGTCACA
TTTCTGCCATTAGAGTGGAACGTTTATCATGGTAATGGCAACACGGATGATTTCT
TCCCTAATCTTAAGTTTGCAACGTATATGAAATTTTTAGCAGCTCGCAAGAAGCC
TAAAATGATTCATTATGCGGGTGAGAACAAACCATGGAATACCGAAAAAGTCGAT
TTTTATGACGACTTTATTGAAAACATCGCTAACACTCCATGGGAGATGGAAATCT
ATAAACGTCAGATGTCGTTAGCGGCTTCGATTGGTTTAACCCATAGCGAGCCGCA
ACAACAAATCTTGTTCCAGACCAAAATCAAGAACGTACTGATGCCTTATGTTAAT
AAATATGCACCAATAGGCACGCCAAGAAGAAACATGATGACTAAATATTATTACA
AAGTACGCCGTGCTATTCTTGGATAA
SEQ ID NO: 24
Klebsiella pneumoniae Glf:
ATGAAAAGAAAAAAAATATTGATCGTAGGCGCTGGCTTCTCTGGTGCAGTTATCG
GTCGCCAACTTGCTGAGAAGGGACATCAAGTCCATATTATCGATCAGCGTGATCA
TATTGGGGGGAATTCTTATGATGCACGCGACTCTGAAACGAATGTGATGGTACAT
GTTTATGGACCCCATATTTTCCATACTGACAATGAATCAGTGTGGAACTATGTCA
ACAAGCATGCAGAGATGATGCCCTATGTGAACCGGGTTAAAGCGACAGTTAATGG
TCAGGTATTTTCCCTGCCTATTAATTTGCATACTATCAATCAGTTTTTCTCAAAA
ACTTGTTCGCCTGATGAGGCCAGAGCGCTCATTGCTGAGAAAGGGGACAGCACTA
TTGCTGATCCACAAACTTTTGAAGAGCAAGCGTTACGCTTTATTGGTAAAGAGTT
ATATGAGGCCTTTTTTAAAGGATATACGATTAAACAGTGGGGGATGCAACCCTCG
GAACTGCCCGCATCTATTCTTAAACGTCTTCCTGTTCGTTTTAACTATGACGATA
ATTATTTTAACCACAAATTTCAGGGCATGCCGAAATGTGGTTATACGCAGATGAT
TAAGTCAATTCTTAAGCATGAGAATATCAAGGTTGACTTACAGCGGGAATTTATC
GTTGACGAGCGAACTCATTACGATCACGTATTCTATAGCGGTCCATTAGATGCGT
TTTATGGCTACCAATATGGCCGTCTGGGCTATCGAACATTAGATTTTAAAAAGTT
TATCTATCAGGGTGATTACCAGGGATGCGCAGTGATGAACTATTGTTCTGTGGAT
GTGCCCTATACTCGCATCACTGAACATAAATATTTTTCTCCCTGGGAACAACACG
ACGGCTCTGTTTGTTATAAAGAGTATAGCCGTGCTTGTGAAGAAAATGATATTCC
TTACTATCCTATTCGCCAGATGGGAGAGATGGCTCTTCTTGAAAAATATTTGTCA
TTGGCCGAGAATGAAACCAACATCACTTTTGTCGGTCGTCTTGGAACCTACCGTT
ACCTTGATATGGATGTGACCATCGCCGAAGCATTGAAAACGGCAGAAGTCTATTT
AAATTCACTCACTGAAAATCAGCCAATGCCTGTGTTTACGGTTTCTGTACGATGA
SEQ ID NO: 25
Klebsiella pneumoniae wbbN:
ATGAAATATACGGCATTGATAGTGACATTCAATCGTCTCGGCAAACTGAAAAAAA
CGGTTGAAGAGACCCTCAAACTTGAATTCACTAATATTGTTATTGTCAATAACGG
GTCCACGGATGGGACCCAAGCCTGGCTTTCGTCAATTGTTGATACACGAGTCATT
GTATTAACCCTCACCGAGAATACCGGTGGGGCGGGGGGCTTTAAAACCGGTAGTC
AGTATATCTGTGAACAGCTGGCAAGTGATTGGGTATTTTTCTACGATGACGATGC
TTACCCCTATCCAGACACGTTGAAGTCCTTTTCACAGCTGGATAAGCAGGGATGT
CGGGTATTTAGTGGACTGGTGAAAGATCCGCAAGGAAAACCGTGTCCGATGAATA
TGCCGTTCTCGCGTGTGCCAACTTCCCTTGGCGACACTGTACGCTATTTACGCTA
CCCTGCAGAGTTTATCCCGGCAGCTAATCGTTCTATGTTCGTACAAACGGTTTCA
TTTGTTGGGATGGTCATACATCGTGATCTGCTCGCGACCAGTCTTGACCACATCC
ATGAACAGCTCTTTATCTACTTTGATGATCTTTACTTTGGCTATCAGCTATCATT
AGCTGGTGAGCAAATTATGTATAGCCCGGAGTTGCTTTTTTATCATGATGTGAGT
ATTCAGGGCAAACTTATTGCCCCTGAATGGAAGGTTTACTATCTATGCCGTAATT
TGATCCTGTCGAAGAAAATATTCCAGAAAAATGCCGTATATAGCAATTCAGCGAT
AGCGATACGCATCCTAAAATATATATTAATCCTGCCATGGCAACGTCAAAAATAT
TCCTATATGAAATTTATTCTTCGTGGAATTTCACATGGCATAAAAGGTATTAGTG
GTAAGTATCATTAA
SEQ ID NO: 26
Klebsiella pneumoniae wbbO:
ATGAGAAAATTGTGTTATTTCATAAATTCGGATTGGTACTTCGATTTACACTGGA
TCGATCGTGCCATCGCCTCCCGTGATGCAGGTTATGAGATTCACATCATCAGCCA
TTTTATTGATGACAACATAATAAATAAATTTAAAACATTTGGCTTTATTTGCCAT
AATGTTACTCTTGATGCTCAATCTTTTAATGCATTAGTTTTCTTTCGTACTTACC
ATGATGTGCAAAAAATTATTAAAAATATAAAACCGGATCTCTTGCATTGCATCAC
TATCAAGCCATGTTTGATTGGTGGTGTGCTCGCGAAGAAATTTAATCTGCCGGTC
ATCGTAAGTTTTGTTGGGCTTGGAAGAGTATTTTCTTCTGACAGCATGCCTTTAA
AATTATTGCGGCAGTTTACTATTGCTGCATATAAATATATTGCCAGTAATAAGCG
CTGTATATTTATGTTTGAACATGACCGCGACAGAAAAAAACTGGCTAAGTTGGTT
GGACTCGAAGAACAACAGACTATTGTTATTGATGGTGCAGGCATTAATCCAGAGA
TATACAAATATTCTCTTGAACAGGATCACGATGTCCCTGTTGTATTGTTTGCCAG
CCGTATGTTGTGGAGTAAAGGACTGGGCGACTTAATTGAAGCGAAGAAAATATTA
CGCAGTAAGAATATTCACTTTACTTTGAATGTTGCTGGAATTCTGGTCGAAAATG
ATAAAGATGCAATTTCCCTTCAGGTCATTGAAAATTGGCATCAGCAAGGATTAAT
TAACTGGTTAGGTCGTTCGAATAACGTTTGCGATCTTATTGAGCAATCAAATATC
GTTGCTTTGCCGTCAGTTTATTCTGAAGGTGTTCCGCGAATTCTTCTGGAAGCAT
CTTCTGTGGGGCGCGCTTGTATTGCTTATGATGTTGGTGGTTGTGATAGCCTTAT
TATTGATAACGATAATGGAATTATTGTTAAAAGCAATTCACCTGAAGAGCTGGCT
GATAAACTTGCCTTTTTGCTTAGCAATCCTAAAGCACGCGTTGAAATGGGTATTA
AGGGGAGGAAACGTATACAAGATAAATTTTCTAGTGTTATGATTATCGATAAAAC
ATTGCAAATATATCATGATGTAGTTCGATGA
SEQ ID NO: 27
Klebsiella pneumoniae gmlA:
ATGCCAAGTTCAGGCCCATTATGGCAACTAATGAAATATGGGTTAGTTGGGATAG
TCAATACACTAATTACGGCAGTTGTAATTTTCCTGCTAATGCATTTGGGTCTTGG
CATTTATCTGTCCAATGCGATGGGTTATGTTGTAGGTATTGTTTTCAGCTTTATA
GCAAACACAATATTTACATTTACGCAACCAATCAGTATCAATAGACTAATAAAAT
TTTTATGTGTTTGCTTCATTTGTTATGTGGCAAATATCATTGTCATAAAAATATT
TTTCGTTTTTATGCCAGAAAAAATATATTCAGCACAAATCCTTGGGATGTTCACA
TACACTATCACAGGTTTTATTTTAAACAAGTTCTGGGCGATGAAATGA
SEQ ID NO: 28
Klebsiella pneumoniae gmlB:
ATGACAACCTCAACTGATATAAAAAGCACTCCTTCTTTAGCTATTGTGGTACCTT
GCTATAATGAACAAGAGGCTTTTCCTTTCTGTCTCGAAAAGCTTTCGAATGTACT
AAATTCATTGATAGCCAGAAATAAAATTAATAACAATAGTTATCTTTTGTTTGTC
GATGATGGTAGTCGTGACAATACTTGGGCACAAATTAAAGATGCCTCGACCGCTT
ATCACTATGTGCGAGGAATAAAATTATCAAGAAATAAAGGACATCAAATTGCGTT
GATGGCAGGGTTACGCTCGGTCGATACAGACGTAAGCATTAGCATCGATGCGGAT
CTACAAGACGATGTAAATTGCATCGAAAAAATGATTGACGCTTACAGCCAGGGAT
ATGACATAGTATACGGCGTAAGAGGTAATCGAGACAGTGACACGTTTTTTAAACG
TACAACAGCTAATGCATTTTACGCAATAATGTCCCACTTGGGAGTAAATCAAACT
CCAAATCATGCAGATTATCGATTATTAAGTAATCGAGCATTGGAGGCTCTTAAAC
AATATAAAGAGCAAAATATATATTTACGTGGATTAGTGCCTCTTGTGGGATACCC
CTCGATCGAGGTGCAATATAGCCGTGAAGAAAGAATTGCAGGTGAATCAAAATAT
CCAATTAAAAAAATGCTTGCGCTGGCTCTCGAGGGAATTACCTCATTATCAGTTA
CACCGTTACGAATTATAGCTATGACAGGTTTTATAACTTGCATCATATCAACCAT
CGCTGCGATTTATGCTTTAATTCAAAAAACAACAGGTACTACAGTTGAGGGATGG
ACATCAGTCATGATCGCTATATTCTTTCTTGGCGGCGTGCAAATGCTTTCTTTAG
GTATTATAGGAGAATATGTCGGAAAAATTTATATAGAGACGAAAAATAGACCTAA
ATATTTCATTGACGAAAGCGTAGGTAATGATAGCAATGGAAAATAA
SEQ ID NO: 29
Klebsiella pneumoniae gmIC:
ATGCAAAATCTGATCAATCCTTTAGCAGAGGGAAATAAAAAAAACGTTTACATTT
TTTATTTCTTTTTGCTTATGTTAACATTTTCACCGGTAATTTTCTTTTCATATGC
ATTTTCAGACGACTGGTCAACACTCTTTGATGCTATAACAAGAAACGGCTCTTCG
TTTCAGTGGGATGTCCAATCTGGTCGTCCCGTTTATGCTGTGTTCCGTTACTATG
GAAAAATGTTAATTAATGATATTTCTTCATTTTCGTATTTGCGGCTTTTTAATAT
ATTAAGTCTTGTTGTCTTAAGTTGTTTTATTTACAACTTCATAGACAGCAGAAAA
ATATTTGATAACCCCGTATTCAAAATAACATTTCCGCTGTTAATTTGCTTACTCC
CTGCGTTTCAAGTTTATGCTTCATGGGCAACATGTTTCCCGTTCACTATTTCAGT
ATTGCTGGCAGGTATTAGTTATAATAAATGTTTCCCACATTCGAAGCAGCGGTCG
TCATTGTCAGAAAAATTAGCATCCATTGTTGTCTTATGGGTGGCATTTGCAATAT
ATCAACCGACAGCAATTACATTCTTATTCTTTTTTATGCTTGATAGTTGTATAAA
AAAAGAAAGTAGTTTAACTGTGAAAAAAGTTGCGACATGTTTTATCATTTTAGTT
ATCGGTGTTGCAGGCAGTTTTATAATGTCAAAAGTACTTCCTGTCTGGCTATATG
GGGAATCATTATCGAGAGCCGAGTTAACCGCAGATATCGGTGGAAAGATGAAATG
GTTCATAAATGAATCACTAATAAACGCTGTAAATAACTATAACATACAACCAGTA
AAAATATATTCTTGGTTCTCCTCGCTTGCAATTTTAATCGGCTTATACACTATTT
TTGTGGGAAAATCAGGCAGATGGAAAACGTTCATAGTCATAGCGATCGGGATAGG
TTCCTACGCTCCAAATTTAGCGACAAAAGAGAATTGGGCAGCATTCCGCTCGTTA
GTGGCCTTAGAACTTATTATATCAACTCTATTTCTTATTGGCATAAATAGCCTTG
TCAGTAGAATTTTTAAGCAAGCATTTGTCTGGCCTCTTATCGCTTTAACAATTAT
GATAATAGCTCAGTATAATATTATAAATGGATTTATTATTCCTCAACGCTCTGAA
ATTCAGGCACTTGCTGCGGAAATAACTAATAAAATACCTAAGAATTACACAGGAA
AATTAATGTTCGATCTCACAGATCCCGCTTACAATGCCTTTACAAAAACACAGAG
ATATGATGAATTTGGGAATATTTCATTAGCAGCACCCTGGGCGCTCAAAGGTATG
GCTGAAGAGATCAGAATTATGAAAGGATTTAATTTCAAACTATCTAACAACGTTA
TAGTTTCTGAGACCAATCGATGTATTGATGATTGTATGGTTATCAAAACGTCAGA
TGCAATGCGAAGGTCAACGATAAATTATTAG
SEQ ID NO: 30
Klebsiella pneumoniae wbbY:
ATGAAGAAAATTCTTATAATGACGCCGGACATTGAGGGGCCTGTCCGTAACGGCG
GTATTGGTACTGCTTTCACTGCCCTTGCCACTACTTTGGCAAAAAAGGGGTATGA
TGTTGATGTATTGTATACATGTGGCGACTATTCTGAATCATCTGTATCGAAATTT
AGCGACTGGTCACGTATTTATAGTACCTTTGGTATCAATCTGCTAAGAACCGGAC
TGATAAAAGAGATTAATATTGATGCACCGTATTTTAGAAGGAAAAGTTATTCAAT
TTATCTCTGGTTGAAAGAAAATAACACCTATGACACTGTTATTTCTTGTGAGTGG
CAGGCAGATCTTTATTACACTTTATTAAGCAAAAAGAATGGAACGGATTTTGAAA
ATACAAAGTTCATTGTAAATACTCACAGTTCAACGTTATGGGCTGATGAAGGTAA
TTACCAGCTTCCATATGATCAGAACCATCTTGAACTCTATTATATGGAGAAAATG
GTGGTTGAAATGGCGGATGAAGTTGTTAGTCCGTCTCAGTATTTAATTGATTGGA
TGTTGAGTAAGCACTGGAATGTTCCTGAAGAACGTCATGTAATTTTAAATTGCGA
GCCATTTCAAGGGTTTGTGACGAGAGATGATGTTACAGTTAAAATAAATGAAAAG
CCAGCTTCTGGCGTTGAGCTTGTATTTTTCGGCCGCCTTGAAACCCGTAAAGGAC
TTGACATATTCCTGCGTGCATTAAGAAAACTATCTGATGAAGATAAAGAGAGCAT
TTCTGGAGTAACCTTCCTCGGAAAAAATGTCACTATGGGGAAAACTGATTCATTT
ACTTATATTATGAATCAGACTAAAAATTTGGGACTCGCAGTTAATGTCATCAGCG
ACTATGATCGTACCAACGCTAATGAATATATAAAAAGAAAAAATGTATTAGTCAT
CATTCCATCACTTGTAGAAAACTCACCCTATACTGTTTATGAATGTTTGATTAAT
AACGTTAATTTTCTCGCTTCAAACGTTGGTGGAATTCCAGAGCTTATTCCGCAGG
AGCATCATGCGGAAGTTCTATTTATTCCTACACCTGCCGATTTATACGGAAAAAT
CCACTATCGCTTAAAAAATATAAATATAAAACCAGGGCTTGCTGAATCACAAGAC
AATATTAAAGAAGCTTGGTTTGTCGCAGTTGAACGAAAAAACAACCGCACATTCA
AGAAAATCGATGAAGCTAACAGCCCGTTAGTTAGCGTGTGTATAACTCACTTCGA
ACGTCACCATTTGCTTCAGCAAGCACTCGCATCAATAAAATCTCAGACGTACCAA
AATATTGAGGTCATCTTGGTTGATGATGGAAGTACGACAGAAGATTCTCATCGTT
ATTTGAATCTCATCGAGAATGATTTTAACTCTCGAGGCTGGAAAATTGTCCGTAG
TTCTAATAACTATCTGGGTGCTGCAAGGAATTTGGCTGCGCGACACGCCTCTGGC
GAATATCTGATGTTTATGGACGATGATAATGTTGCTAAGCCTTTTGAGGTAGAAA
CGTTTGTTACTGCAGCATTAAACTCTGGGGCCGATGTGTTAACCACACCAAGCGA
TCTTATTTTTGGTGAGGAGTTCCCTTCTCCGTTCCGTAAAATGACGCACTGCTGG
CTTCCGTTAGGGCCTGATTTAAATATCGCCAGCTTTAGTAACTGCTTTGGCGATG
CTAATGCGCTGATCAGAAAAGAGGTTTTCGAAAAAGTAGGCGGATTTACTGAAGA
TTACGGTTTAGGTCATGAAGACTGGGAGTTTTTTGCCAAAATATCATTACAGGGA
TATAAATTGCAAATCGTCCCGGAACCTCTATTTTGGTATAGAGTTGCAAACTCCG
GCATGTTGTTAAGTGGAAATAAGAGTAAAAATAACTACCGCAGTTTCCGTCCTTT
TATGGATGAGAATGTTAAATATAACTATGCAATGGGGTTGATACCTTCCTACCTC
GAGAAGATTCAAGAACTTGAGAGTGAAGTGAATCGCTTGCGGAGCATCAATGGTG
GTCATTCTGTCAGTAACGAGTTACAACTTTTAAATAATAAGGTTGATGGTCTTAT
TTCTCAGCAAAGAGATGGCTGGGCCCATGACCGTTTTAATGCTCTGTATGAAGCA
ATTCATGTCCAAGGCGCAAAACGAGGCACCAGCCTGGTTCGCCGGGTTGCCCGGA
AAGTGAAATCAATGTTAAAATAA
SEQ ID NO: 31
Klebsiella pneumoniae wbbZ:
ATGACCAATATGAAGTTAAAATTTGATTTGCTTCTAAAATCTTATCATCTATCTC
ATCGATTTGTCTATAAGGCAAACCCTGGTAATGCTGGTGATGGTGTAATTGCATC
TGCGACGTATGACTTTTTTGAACGAAATGCTCTTACCTATATCCCTTACAGAGAT
GGCGAGCGCTACAGTTCTGAAACTGATATTTTAATTTTTGGAGGCGGAGGAAACC
TGATAGAAGGATTGTATTCTGAAGGTCATGACTTTATCCAGAATAATATTGGGAA
GTTTCATAAAGTAATAATAATGCCGTCGACAATCAGAGGGTATAGCGATTTATTC
ATCAACAATATTGATAAGTTTGTTGTTTTTTGTCGCGAAAATATCACCTTCGATT
ATATTAAATCTCTCAACTACGAACCAAACAAGAACGTATTCATTACTGATGATAT
GGCATTTTATCTCGATCTTAATAAATACCTGTCACTTAAACCCGTCTATAAAAAA
CAGGCCAACTGCTTCAGAACGGACTCCGAATCTCTAACTGGAGACTACAAAGAAA
ACAATCATGATATTTCGCTCACCTGGAATGGCGATTATTGGGATAATGAATTTCT
GGCGCGTAATTCTACCCGTTGCATGATAAACTTTCTTGAAGAGTATAAAGTTGTC
AATACCGACAGGCTGCATGTGGCAATTTTAGCATCTCTGCTTGGCAAAGAAGTCA
ACTTCTATCCTAACTCATATTACAAAAATGAAGCTGTTTACAATTATTCACTTTT
TAATCGTTATCCAAAAACATGCTTTATTACGGCAAGTTGA
SEQ ID NO: 32
Klebsiella pneumoniae manC:
ATGTTGCTTCCTGTGATCATGGCTGGTGGTACCGGCAGTCGTCTCTGGCCGATGT
CTCGCGAGCTTTACCCGAAACAGTTCCTCCGCCTGTTCGGGCAGAACTCCATGCT
GCAGGAAACCATCACCCGACTCTCGGGCCTTGAAATCCATGAACCGATGGTCATC
TGTAACGAAGAGCACCGCTTCCTGGTGGCCGAACAGCTGCGCCAGCTCAACAAGC
TGTCGAACAACATTATTCTTGAGCCGGTCGGGCGCAACACCGCCCCGGCCATCGC
CCTGGCGGCCCTCCAGGCCACCCGCCACGGCGACGACCCGCTGATGCTGGTCCTC
GCCGCCGACCATATCATCAATAACCAGCCGGTCTTCCACGACGCCATCCGCGTCG
CCGAGCAGTATGCCGATGAAGGCCATCTGGTCACCTTCGGTATCGTGCCGAACGC
CCCGGAAACCGGCTACGGCTACATCCAGCGCGGCGTGGCCCTCACCGACAGCGCC
CACACCCCGTACCAGGTGGCCCGCTTCGTGGAGAAGCCGGACCGCGAGCGCGCCG
AGGCCTACCTCGCCTCCGGGGAGTACTACTGGAACAGCGGCATGTTTATGTTCCG
CGCCAAAAAATACCTCTCCGAGCTGGCCAAATTCCGCCCGGATATCCTCGAAGCC
TGCCAGGCCGCGGTCAATGCCGCCGATAACGGCAGCGACTTCATCAGCATCCCGC
ATGACATTTTCTGTGAGTGCCCGGACGAGTCCGTGGACTACGCGGTGATGGAGAA
AACCGCCGACGCGGTGGTGGTCGGTCTCGATGCCGACTGGAGCGACGTCGGCTCC
TGGTCCGCCCTGTGGGAGGTCAGCCCGAAAGATGAGCAGGGTAACGTCCTCAGCG
GCGACGCGTGGGTGCACAACAGCGAAAACTGCTACATCAACAGCGACGAGAAGCT
GGTGGCGGCCATCGGCGTGGAGAACCTGGTGATTGTCAGCACCAAGGACGCCGTG
CTGGTGATGAACCGTGAGCGTTCCCAGGACGTGAAGAAGGCGGTCGAGTTCCTCA
AGCAGAACCAGCGCAGCGAGTACAAGCGCCACCGCGAGATTTACCGTCCCTGGGG
CCGCTGCGACGTGGTGGTCCAGACCCCGCGCTTCAACGTCAACCGTATTACGGTG
AAACCGGGGGGCGCCTTCTCGATGCAGATGCACCACCACCGTGCCGAGCACTGGG
TCATTCTCGCCGGCACCGGCCAGGTGACGGTCAACGGCAAGCAGTTCCTGCTGAC
CGAGAACCAGTCCACCTTTATTCCGATTGGCGCCGAGCACAGCCTGGAAAACCCG
GGCCGCATTCCGCTGGAAGTGCTGGAGATCCAGTCGGGGTCGTACCTCGGCGAGG
ACGACATTATTCGTATTAAAGACCAGTATGGTCGTTGCTAA
SEQ ID NO: 33
Klebsiella pneumoniae manB:
ATGACACAGTTAACATGCTTTAAGGCTTATGACATCCGTGGTGAACTGGGCGAGG
AGCTGAACGAGGACATCGCCTACCGTATCGGCCGCGCCTATGGCGAATTTCTGAA
ACCCGGGAAGATAGTGGTGGGGGGCGATGTGCGCCTCACCAGCGAGTCGCTGAAG
CTGGCGCTGGCCCGCGGGCTGATGGACGCCGGCACCGACGTGCTGGATATTGGCC
TGAGCGGCACGGAAGAGATTTACTTCGCCACTTTCCACCTCGGGGTGGACGGCGG
TATCGAGGTGACGGCGAGCCATAACCCGATGAACTACAACGGCATGAAGCTGGTG
CGCGAGAACGCGAAGCCCATCAGCGGCGACACCGGCCTGCGGGATATCCAGCGCC
TGGCGGAGGAGAATCAGTTCGCGCCGGTAGACCCGGCGCGTCGCGGGACCCTGCG
CCAGATATCGGTGCTGAAGGAGTACGTCGACCACCTGATGGGCTATGTGGACCTG
GCGAACTTCACCCGTCCGCTGAAGCTGGTGGTGAACTCCGGCAACGGGGGGGGGG
GGCACGTGATTGATGAAGTGGAGAAACGCTTCGCGGCGGCCGGGGCGCCGGTGAC
CTTTATCAAGGTGCATCACCAGCCGGACGGCCATTTCCCGAACGGTATCCCGAAC
CCGCTGCTGCCGGAGTGCCGCCAGGACACCGCCGACGCGGTGCGTGCGCATCAGG
CGGACATGGGGATCGCCTTTGACGGCGACTTCGACCGCTGCTTCCTGTTCGATGA
CGAGGCGTCGTTTATCGAGGGGTACTACATTGTCGGCCTGCTGGCGGAGGCGTTC
CTGCAGAAACAGCCGGGGGCGAAAATCATTCACGACCCGCGTCTGACGTGGAACA
CGGTGGACATCGTGACCCGCAGCGGCGGCCAGCCGGTGATGTCGAAGACGGGGCA
TGCGTTCATCAAGGAGCGGATGCGCCAGGAAGACGCCATCTACGGCGGGGAAATG
AGTGCGCACCATTACTTCCGCGACTTCGCCTACTGCGACAGCGGGATGATCCCGT
GGCTGCTGGTGGCGGAGCTGCTGTGCCTGAAGAACAGTTCGCTGAAATCGCTGGT
GGCGGACCGCCAGGCGGCGTTCCCGGCGTCGGGGGAGATCAACCGCAAGCTGGGG
AATGCGGCGGAGGCGATAGCGCGCATCCGGGCGCAGTATGAGCCGGCCGCCGCAC
ACATCGACACAACGGACGGTATCAGTATTGAATACCCTGAGTGGCGCTTTAACCT
GCGCACGTCCAACACGGAGCCGGTGGTGCGTCTGAACGTTGAGTCCAGAGCGGAT
ACTGCGTTAATGAATGAGAAAACCGCCGAGCTGCTCAACCTGTTAAAAGAGGAAT
CGCTTTGA
SEQ ID NO: 34
Klebsiella pneumoniae wzm:
ATGTTTTCAGCGATCTATCGCTACCGTGGCTTTATTATTGACAGCGTCAAACGGG
ACTTTCAGTCCCGTTACCAGACTAGCTTCTTAGGCGCGGCATGGCTGATCTTACA
GCCGATCGCCATGATTTCCGTATATACATTAATCTTTTCTGAGTTAATGCGTGCC
CGCCTGGCGGGCATGGACGGCCCTTTTGCCTACAGTATCTACCTCTGTTCCGGGG
TGTTAACCTGGGGGCTGTTTACGGAAACGCTCGGCAATCTGGTCAACGTTTTTCT
GACCAACGCCAACATTCTTAAAAAGCTTAGCTTTCCGCGGATCTGTTTACCGATC
ATTGTCACCGCCTCGGCGTTCATTAACTTCCTGATCATTTTTGGTCTGTTTGTAC
TGTTTCTGATCGTCACGGGCAATTTCCCGGGCATGATTTTCTTTGAAATCATTCC
GGTGCTGATCGTTCAGATGCTGTTCACCCTCGGCCTCGGGATCATCCTCGGGGTG
CTGAACGTTTTTGTCCGCGACGTCGGGCAGTTCGTGAATATCCTGCTGCAGTTTT
GGTTCTGGTTTACGCCCATTGTCTACGTGTCCAAAACGCTGCCGGAGTGGGTCTC
TGGTCTGCTGGCGTATAACCCGATGGCGACCATTATCGGTTCATACCAGAACGTG
ATGCTCTATCACCAGAGCCCTAACTGGCTGGCGCTGCTTCCGGTCACGGTGCTGT
CCGTCATTCTGTTTTTATTTGCCTGGCGTTTATTTAAAAAACATGCCGCTGATAT
TGTGGACGAGATTTAA
SEQ ID NO: 35
Klebsiella pneumoniae Wzt:
ATGAGTATCAAAGTTCAGCACGTCGGCAAGGCGTATAAATATTATCCCTCCAAAT
GGAACCGGGTCATTGAGAAACTTCTGCCGGGCGATAAGCCGCGGCACAGCAAGAA
ATGGGTATTGAAAGATATCAATTTCAGTATTGAACCCGGTGAAGCGGTCGGCATT
GTTGGGGTGAACGGCGCAGGTAAAAGTACGTTACTGAAGCTGCTGACTGGCACCA
CTCAGCCGACCAAAGGCAGCATTGAGATCCAGGGGCGTGTCGCTGCGCTGCTGGA
GCTGGGCATGGGCTTCCATCCTGACTTTACCGGTCGGCAGAACGTGTATATGTCC
GGGCTGATGATGGGCCTGAGCCGGGAAGAGATTGAGCGCTTAATGCCGGAGATCG
AAGCCTTTGCGGATATCGGTGACTACATTGAAGAGCCCGTGCGCATCTACTCCAG
CGGGATGCAAATGCGCCTGGCGTTCGCCGTGGCCACGGCCTCACGCCCGGATATT
CTGATCGTCGATGAAGCGCTTTCCGTTGGTGACTCCCGCTTTCAGGCGAAGTGCT
ATGCCCGTATTGCGGACTTCAAAAAGCAGGGCACCACGCTGCTGCTGGTCTCCCA
CAGCGCCGGGGATATCGTCAAACACTGTGACCGCGCCATTTTCCTCAAAAATGGT
GATATCTGTATGGACGGCACCGCCCGTGACGTGACCAACCGTTATCTGGATGAGC
TGTTTGGCAAAGCCGACAAAAACAGCGCGCCAAAAAGCGAAACGGCAACCTCGTC
AGCCAGCGGCGAAAGTCAGATGTCTCTCGATGAGATTGAAGATGTGTACCACACG
CGCCCAGGCTACCGTCCGGAAGAGTACCGTTGGGGGCAGGGGGGTGCAAAAATCA
TTGATTATCACATCCAAAGCGCCGGGGTTGATTTTCCGCCTTCACTGACGGGCAA
TCAGCAGACCGATTTCCTGATGAAAGTCGTATTTGAATATGACTTTGATTGCGTG
GTACCGGGTTTGTTAATCAAAACTCTGGATGGCTTATTTCTATATGGTACCAACT
CTTTCCTGGCCTCGGAAGGCCGGGAAAACATTTCGGTATCACGTGGGGACGTTAG
AGTATTTAAATTCAGTTTTCCGGTTGATTTAAATAGCGGTGACTATCTTCTGTCG
TTTGGTATTTCAGAGGGAAGCCCGCAAACCGAAATGACGCCGCTCGATCGTCGCT
ATGACTCCATCATTTTGCATGTAACTAAGAGCATGGATTTCTGGGGAGTGATTGA
CCTGAAGTCGACTTTCAATAGTTACAAATGA
SEQ ID NO: 36
Klebsiella pneumoniae wbdD
ATGACTACTAATACACATAAATTGGTTAGCGAATTACCTGAAATTTATCAGACTA
TTTTTGGGCATCCTGAGTGGGATGGCGATGCTGCACGAGACTGTAATGAACGGCT
CGCGCTAATTAGTGAACAATATGACAGCTTGTCCAGAGAGTTAGGAAGGCCACTA
CGGGTTCTCGACCTGGGCTGTGCTCAGGGGTTCTTCAGTTTAAGTTTGGCAAGCA
AGGGTGCCAGCGTATTAGGTATCGACTTTTTGCAGCAGAACATTGATGTTTGTCA
GGCGCTTGCTGAAGAAAATCCACATTGTGATGTTAAATTTCAAGTCGGGCGGATA
GAAGACATTGTCAGCACTCTGGAAGAAAACCAATTTGATCTCGCCATTGGACTAA
GTGTTTTTCACCACATTGTTCATCTGCATGGGGTTGCTGAAGTCAGATCGCTGTT
AGAGCGTTTGGCAAATCTGACGCAGGCGATGATTCTCGAGCTCGCTGTCAAGGAG
GAACCACTCTATTGGGGGAAATCTCAGCCTGAAGATCCGCGTGAACTTATTGACC
AATGTGCTTTCTATCGATTGATTGGAAGATTTGACACTCATCTGTCTAATATTTC
ACGTCCGATGTATATTATCAGTAACCACAGGGTTATTCTTCCGGAATTTAATCAG
CCTTTTACTTCATGGCGCGACAGTCCTTACACCGGAGCAGGCTTTGCGCATAAAC
AGAGCCGTCGCTATTATTTCTCTTCGGAGTTCATATGTAAGTTCTATCGTTTTAG
TACAGTAAGTTGCTTACTAACTGATAAGGAGAGCGAGCGTAATCGTACTGAACTC
GCCCATGAAGAAGCTTTTCTTAAATCTCCACCATCTGGCTTAAAAGTGCCGGCGT
TGTTTACTGCAGGGGGGAATGGAGAAGCGGGATGGTTGGTAATGGAAAAAATTCC
CGGAGAGCTGTTAAACGACGTTCTGGCCAGTGAACGGCATATTGATCGGGAAAAA
GTTATTTCCGATCTCCTCGACCAATTAGTTATTTTGGAAGAACATGGTCTATATC
ATGATGATTTCAGAACATGGAATGTTTTAATTGACGATAATGACAGCGCTCGTTT
AATAGATTTTGGTTCGATTGGCGATGTACAACAAGACTGCAGCTGGCCAGTTAAT
ATTTTCCAGTCGTTCATTATTTTTGTAAATGAAATATTTTGTGAAAATAAATCCT
GGAGGGGCTTCTGGCGTTCCGCACCATTAAGTCCTTTCCAGTTGCCTGAACCGTA
TTCAAATTGGTTGACAGCATTCTGGAAACATCCTGTTGGTGAGTGGAGTTTTGCT
TTACTCCAACAACTCTTTTCAACCAAAGATGCTCTACCGGCTGCGAGTTCCATTA
TGGACGCTTCTGATCTATGGGTCCGGGCTCAGGAGCCCGTATTGTTGGAAAGTCA
AACGCAAATACGCAATACGGATGCGCGGGTAGTCCGTCTCGAGTCGCAAATCAAT
GAACTCACCTCCCTGATTAATATTATGGGTGAGAGCATTCAGACGTTTGAGAAGC
GTGAGTATCCGCCACAAGACGTTACTACTAATGTACAGCCGCGTATCGAGATTGA
GCAGAGTAAAGCCGTTGATTCAGAAGAGATTATGCGACTTCATACGCAGCTCAAT
GATGCTCAGCAAGAAATAGAGAATCTACGTCATGAGATTGCTAAAATTCATTATA
GTCGCTCATGGAAAATGACCAAGTGGTATCGGTACGCTGGCTTACAGTACTATCT
GCTTCGTCAGTACGGCTTCAAACAGCGTTTTAAGCATTTACTCAAACGAGTGCTT
AGCAACGTAATTTATTTTTTGCGTGCACATCCACGACTAAAGCAGAAGGTGATCA
ATCTACTGCGTACAATTGGAATTTATGACTTTGCTTATCGTATGCATCGTCGTAT
GAATCCTGGTTCACATAACCCTTATCCAAACGACCCACAATACCAGTCGCAGACT
GAAAAGCAGATCTTACATCCAGAGTTATTGCCTCCGGAAGTTAACTCAATTTTTA
GCGAGCTTAAAAACAAAAGATAA
SEQ ID NO: 37
Klebsiella pneumoniae wbdA:
TTGCATATTTTGATTGACGTACAAGGATATCAATCGGAAAGTAAATTCCGTGGAG
TTGGTCGCAGCACCTATGAAATGAGTCGTGCGATCATAAAAAATGCTGGCCAGCA
TCGAGTAAGCATTTTAATGAATGGCATGTATTCGATTGATAGTATAAATGAAATT
AAAAAAAGCTGGGGTGATATATTACCGCAGGAAGAAATGTTTATTTTTTCAGCTG
CTGGCCCTACAGCTCTTCGCGACTGTGAAAACCATCCCCGGAGTGTTGCCGCCAC
ACTAGCTCGTGAACTTGCTATTGCTAATATCAATCCCGACGTTGTTTTTATTATT
AATTTCTACGAAGGTTTTGACGATAGTTATACCGTCTCAATTCCTCAAACTACAG
TACCATGGAAAACAGTTTGTGTTTGTCACGATCTAATTCCGTTACTGAATAAAGA
ACGCTATCTGGGCGAACCAAACTTCCGTCAGTATTATTATGATAAACTAGCTCAA
TACGAAAGGGCGGACGCTATTTTTGCTATTTCCAGATCATCCATGCAGGAAGTTA
TCGATTACACATCGATTCCGGCAGAAAAAATTATTAATATTTCATCTGGAGTAAG
CGATTCATTTAAAATTAAAGATTATACTCACGATGAAATCAAAGACTTACGTAAT
AAATATCATCTTCCTCAAGAGTTTATTCTTTCTTTGGCAATGATAGAGCCACGTA
AAAATATTGAAGCGCTGATTCATGCATATAGTTTATTACCGCATGCCCTGCAACA
GAGTTATCCCTTAGTTTTAGCCTATAAAATTAGCACCGATGAAAAGGAAAGGCTG
TACCGAGTTGCAGAGAACTATGGTTTATCTCGTAATCAGCTTATTTTTACAGGCT
TCTTAAACGATAGTGACCTTATCGCACTTTACAATTTGTGCAAAATTTTCGTTTT
CCCCTCTATACATGAAGGGTTTGGCCTGCCGCCACTAGAAGCTATGCGTTGTGGT
GCAGCTACGCTGGGTTCAAATGTGACCAGCTTACCCGAGGTCATCGGTATGGAAG
AGGCTTTATTTAATCCTCTGGATGTCCCCGACATTTGCCGTGTTATGCAAAGGGC
CTTGACTGACAGTGAGTTCTACTCAGCATTAAAAGCTCATGCTCCGGCGCAGGCG
GCAAAGTTCACATGGGATCACACCGCGCAGCTCGCGTTAAAGGGATTTGAGAGGC
TTGTAGATAAGGCTTCCGCATCAGAACCTCTGGATATCACAAGCTTCACCGCATA
CACCATTAATAGAATTAAAAATATTGCAGAATTAAGTGAAACCGAACGCTTACAG
ACAGCCTGGGCGATTGCTCGTAATAGCTTTGCTACACATCAGCGCAAGCTGCTGG
TTGATATTTCTGTTCTTGTTGAGCATGATGCGAAAACGGGAATTCAACGGGTTTC
TCGCAGTATACTTAGTGAATTACTGAAATCTGGCGTTGCTGGTTATACTGTCAGT
GCGGTTTATTATCGACCGGGTGAATGCTATCGCTATGCCAACGAATACCTGAATA
CCCATTTTAACGGGGCGTTCGGGCCTGATGTACCTGTACTGTTTACCAAAGATGA
TATTCTGGTTGCTACCGATCTAACTGCCCATCTGTTTCCTGAGCTTACTGTCCAG
CTGGATTTTATTCGTCTATCCGGTGCCAAGGTTTGTTTTGTTGTGCATGACATTT
TGCCTCTGAGAAGACCGGAGTGGAGCGATGAGGGAATGCAACGCGTGTTCCCCAT
TTGGTTATCTTGCATTGCGCAGCACGCAGACCGCTTGATTTGTGTATCAGCAAGC
GTTGCAGAGGATGTAAAAGCCTGGATTGCGGAAAACAGCCATTGGGTGAAACCGA
ACCCGCTGCTGACCGTCAGCAACTTCCATCTGGGAGCCGACCTCGATGCCAGCGT
ACCGTCCACTGGCATGCCGGATAATGCCCAGGCGCTGTTAGCAGCGATGGCCGCG
GCTCCATCATTTATCATGGTGGGCACGATGGAACCACGCAAAGGACATGCGCAGA
CGCTAGCGGCATTTGAAGAATTGTGGTTACAGGGCAAGAACTACAATCTGTTTAT
CATTGGTAAACAGGGGTGGCATGTTGATGATTTATGTGAACGTTTACGTCACCAT
CCACAGCTAAATAAAAAACTATTTTGGCTACAAAACATTAGCGATGAGTTCCTTA
CGAAGTTGTATTCTCAGTCTAGTGCGTTAATCTTCGCATCTCTCGGAGAAGGCTT
TGGCCTGCCGTTGATTGAAGCGGCGCAGAAAAAGCTGCCGGTGATTATCCGTGAC
ATTCCGGTGTTTAAAGAGATTGCTCAGGAACATGCGTGGTATTTCTCCGGGGAAG
CGCCGGCCGACATCGCGAAGGCCGTCGAAGACTGGTTAGCCCTGTATGAGCAAAA
CGCGCATCCTCGTTCCGAGAATATCAACTGGTTAACCTGGAAGCAGAGCGCGGAA
TTTCTCCTGAAAAACCTGCCGATTATCGCGCCAGCCGCGAAGCAATAA
SEQ ID NO: 38
Klebsiella pneumoniae wbdB:
ATGAAAATTATTTTTGCTACTGAGCCAATTAAATACCCGTTAACGGGCATCGGTC
GGTATTCCCTGGAGCTGGTTAAGCGGCTGGCGGTCGCCCGCGAAATCGAAGAGCT
GAAGCTGTTTCACGGCGCGTCGTTTATCGATCAGATCCCCCAGGTGGAGAATAAA
AGCGATACCAAAGCCAGCAATCATGGTCGTTTGTCGGCGTTTCTGCGCCGCCAGC
CGCTGCTGATTGAGGCGTATCGCCTGCTGCACCCGCGGCGCCAGGCGTGGGCATT
GCGCGACTATAAAGATTATATCTACCATGGTCCCAATTTTTACCTGCCGCATCGC
CTGGAACACGCCGTGACCACGTTTCATGACATCTCCATTTTTACCTGCCCGGAAT
ATCATCCAAAAGATCGGGTTCGCTATATGGAGAAGTCCCTGCATGAGAGCCTGGA
TTCGGCAAAGCTGATCCTGACCGTCTCTGACTTCTCGCGCAGTGAAATCATCCGC
CTGTTCAACTATCCGGCGGAGCGGATCGTCACCACCAAGCTGGCCTGCAGCAGCG
ACTATATTCCACGCAGCCCGGCGGAGTGCCTGCCGGTCCTGCAGAAATATCAGCT
GGCGTGGCAGGGGTATGCGTTATATATCGGCACCATGGAGCCGCGTAAAAATATC
CGTGGTCTGCTGCAGGCCTATCAGCTGCTGCCGATGGAGACCCGCATGCGCTACC
CGCTGATCCTCAGCGGCTATCGCGGCTGGGAAGACGATGTGCTGTGGCAGTTAGT
CGAGCGTGGTACGCGTGAAGGGTGGATCCGTTACCTGGGCTATGTCCCGGATGAG
GACCTGCCTTATCTGTACGCGGCGGCCAGAACCTTTGTTTATCCCTCCTTCTATG
AGGGATTCGGTTTACCTATTCTTGAAGCGATGTCTTGCGGTGTGCCGGTAGTATG
TTCCAATGTCACTTCTTTGCCTGAGGTGGTTGGCGATGCCGGCCTCGTTGCCGAT
CCTAATGATGTAGACGCGATTAGCGCGCATATTTTGCAGAGCCTGCAGGATGATA
GCTGGGGGGAAATCGCCACCGCGCGCGGTCTTGCCCAGGCGAAACAGTTTTCGTG
GGAGAACTGTACGACCCAGACCATTAACGCCTATAAATTACTCTAA
SEQ ID NO: 39
Klebsiella pneumoniae wbdC:
TTGAGAGTTCTACACGTCTATAAGACCTACTATCCCGATACCTACGGCGGTATTG
AGCAGGTCATTTATCAGCTCAGTCAGGGTTGCGCCCGCCGGGGGATCGCAGCCGA
TGTTTTTACTTTTAGCCCGGACAAAGAGACAGGTCCTGTCGCCTACGAAGACCAT
CGGGTCATTTATAATAAGCAGCTTTTTGAAATTGCCTCCACGCCGTTTTCGTTGA
AAGCGTTAAAGCGTTTTAAGCAGATTAAAGATGATTACGACATCATCAACTACCA
TTITCCGTTTCCCTTTATGGATATGCTGCATCTCTCGGCGCGGCCTGACGCCAGA
ACGGTGGTGACCTATCACTCGGATATTGTGAAACAAAAACGGTTGATGAAGTTGT
ACCAGCCGCTGCAGGAGCGATTCCTCGCCAGCGTAGACTGCATTGTTGCCTCGTC
GCCCAACTACGTGGCCTCCAGCCAGACCCTGAAAAAATATCAGGATAAAACGGTG
GTGATCCCGTTTGGTCTGGAGCAGCATGACGTGCAGCACGATCCGCAGCGGGTGG
CGCACTGGCGGGAAACCGTCGGCGATAACTTCTTCCTCTTCGTCGGCGCTTTCCG
CTACTACAAAGGGCTGCACATTCTGCTGGATGCCGCCGAGCGTAGCCGGCTGCCA
GTGGTGATCGTCGGGGGGGGGCCGCTGGAGGCGGAGGTGCGGCGTGAGGCGCAGC
AGCGCGGACTGAGCAATGTGGTGTTTACCGGCATGCTCAACGACGAAGATAAATA
CATTCTCTTCCAGCTCTGCCGGGGCGTGGTCTTCCCCTCGCATCTGCGCTCAGAG
GCGTTTGGCATTACGTTACTGGAAGGCGCGCGCTTTGCCAGGCCGCTGATCTCCT
GCGAGATCGGCACCGGTACCTCGTTCATTAACCAGGACAAAGTAAATGGCTGCGT
GATCCCGCCGAATGACAGTCAGGCGCTGGTGGAGGCGATGAATGAGCTCTGGCAT
AACGATGAAACCGCCAGCCGCTATGGCGAAAACTCGCGTCGTCGTTTTGAAGAGA
TGTTTACAGCCGACCATATGATTGACGCTTACGTCAATCTCTACACTACGCTGCT
GGAAAGCAAATCCTGA
SEQ ID NO:40 PCR primer
5′-GAAGGCGGGCGCGTGACCA TTCTCGGC
SEQ ID NO:41 PCR primer
GCCGAGAATGGTCACGCGCCCGCCTTC

1. An immunogenic composition comprising a Klebsiella pneumoniae O1v1 O-antigen polysaccharide conjugate, a Klebsiella pneumoniae O2a O-antigen polysaccharide conjugate, a Klebsiella pneumoniae O2afg O-antigen polysaccharide conjugate, and a Klebsiella pneumoniae O3b O-antigen polysaccharide conjugate, wherein each of the Klebsiella pneumoniae O1v1, O2a, O2afg, and O3b O-antigen polysaccharides are individually conjugated to a carrier protein, wherein the Klebsiella pneumoniae O3b O-antigen polysaccharide conjugate is present in said immunogenic composition at at least a 2-fold higher saccharide dose than each of the Klebsiella pneumoniae O1v1 O-antigen polysaccharide conjugate, the Klebsiella pneumoniae O2a O-antigen polysaccharide conjugate, and the Klebsiella pneumoniae O2afg O-antigen polysaccharide conjugate saccharide doses.

2. The immunogenic composition according to claim 1, wherein the Klebsiella pneumoniae O3b O-antigen polysaccharide conjugate is present at at least a 4-fold higher saccharide dose than each of the Klebsiella pneumoniae O1v1 O-antigen polysaccharide conjugate, Klebsiella pneumoniae O2a O-antigen polysaccharide conjugate, and Klebsiella pneumoniae O2afg O-antigen polysaccharide conjugate saccharide doses.

3. The immunogenic composition according to claim 1, wherein the saccharide dose of the Klebsiella pneumoniae O3b O-antigen polysaccharide conjugate is at least 6-fold higher than each of the Klebsiella pneumoniae O1v1 O-antigen polysaccharide conjugate, the Klebsiella pneumoniae O2a O-antigen polysaccharide conjugate, and the Klebsiella pneumoniae O2afg O-antigen polysaccharide conjugate saccharide doses.

4. The immunogenic composition according to claim 1, wherein the saccharide dose of the Klebsiella pneumoniae O3b O-antigen polysaccharide conjugate is between 2-fold and 20-fold higher than each of the Klebsiella pneumoniae O1v1 O-antigen polysaccharide conjugate, the Klebsiella pneumoniae O2a O-antigen polysaccharide conjugate, and the Klebsiella pneumoniae O2afg O-antigen polysaccharide conjugate saccharide doses.

5. The immunogenic composition according to claim 1, wherein the saccharide dose of the Klebsiella pneumoniae O3b O-antigen polysaccharide conjugate is between 2-fold and 10-fold higher than each of the Klebsiella pneumoniae O1v1 O-antigen polysaccharide conjugate, the Klebsiella pneumoniae O2a O-antigen polysaccharide conjugate, and the Klebsiella pneumoniae O2afg O-antigen polysaccharide conjugate saccharide doses.

6. The immunogenic composition according to claim 1, wherein the saccharide dose of the Klebsiella pneumoniae O3b O-antigen is 10-25 mg per human dose and wherein the saccharide dose of each of the Klebsiella pneumoniae O1v1 O-antigen, the Klebsiella pneumoniae O2a O-antigen and the Klebsiella pneumoniae O2agf O-antigen is 1-10 mg per human dose.

7. The immunogenic composition according to claim 1, wherein: (i) the conjugate is a bioconjugate, (ii) the volume of the human dose is 0.5 ml, or (iii) both (i) and (ii).

8. The immunogenic composition according to claim 1, wherein the carrier protein is a detoxified Exotoxin A of Pseudomonas aeruginosa (EPA), optionally wherein the detoxified EPA comprises 3 to 7 inserted consensus sequences D/E-X-N-Z-S/T, wherein X and Z may be any natural amino acid except proline, and optionally wherein the detoxified EPA comprises an amino acid sequence that is at least 95% identical to SEQ ID NO: 17.

9. The immunogenic composition according to claim 1, wherein the Klebsiella pneumoniae O1v1 O-antigen polysaccharide has the structure -(D-galactan II)n-(D-galactan I) m-GlcNAc, as follows: 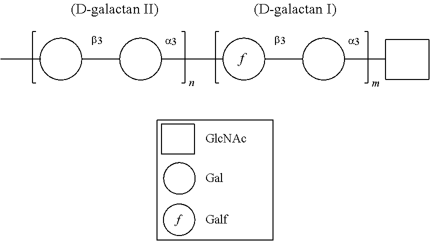
optionally wherein the number of repeat units of D-galactan II (n) ranges from 5 to 7 and the number of repeat units of D-galactan I (m) ranges from 2 to 10 and optionally wherein the ratio of D-galactan II:D-galactan I ranges between 2:1 to 1:50.

10. The immunogenic composition according to claim 1, wherein the Klebsiella pneumoniae O2a O-antigen polysaccharide has the structure -(D-galactan I)n-GlcNAc, as follows: 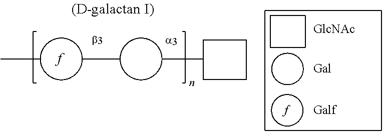
optionally wherein the number of repeat units of D-galactan I (n) ranges from 10 to 30.

11. The immunogenic composition according to claim 1, wherein the Klebsiella pneumoniae O2afg O-antigen polysaccharide has the structure -(D-galactan III)n-GlcNAc, as follows: 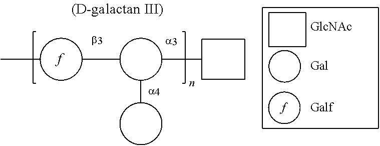
optionally wherein the number of repeat units n ranges from 5 to 25 and optionally wherein the degree of branching ranges from 90-100%.

12. The immunogenic composition according to claim 1, wherein the Klebsiella pneumoniae O3b O-antigen polysaccharide has the structure Me-P-3(Man-α2-Man-α3-Man-α3)n-Man-α3-Man-α3-GlcNAc, as follows: 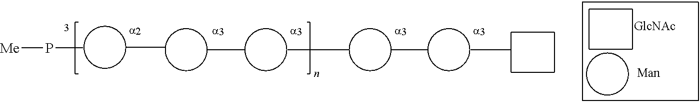
optionally wherein the number of repeat units n ranges from 5 to 25.

13. The immunogenic composition according to claim 1 further comprising a pharmaceutically acceptable excipient or carrier.

14. The immunogenic composition of claim 1 further comprising an adjuvant.

15. A method of inducing an immune response to Klebsiella pneumoniae in a subject, the method comprising administering a therapeutically or prophylactically effective amount of the immunogenic composition of claim 1 to a subject in need thereof.
